SEQUENCE LISTING (33 sequences)

SEQ ID NO: 1 - protein, 174 aa, Homo sapiens
  Human G-CSF
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 123 aa, Artificial Sequence
  boskar_1
AALAAELAEIYKGLAEYQARLQSLEGISPELGPALDALRLDVADFATTLAQAMEEKKTNLPQSFLLKALEQIRKIQADAAALREKLAATYTGTDRAAAAVEIAAQLEAFLEKAYEILRHLAAA

SEQ ID NO: 3 - protein, 123 aa, Artificial Sequence
  boskar_2
AALAAELAEIMKGLQEYQARLKSLEGISPELGPALDALRLDMADFATTMAQMMEENPSDLPQSFLLKALEQIRKIQADAAALREKLAATYPNSQRAAAAVEIAAQLEAFLEKAYQILRHLAAA

SEQ ID NO: 4 - protein, 123 aa, Artificial Sequence
  boskar_3
AALAAVLAEIYKGLAEYQARLQSLEGISPELGPALDALRLDVADFATTIAQAMEENKGPLPQSFLLKALEQIRKIQADAAALREKLAATYPSSQRAAAAVEIAAQLEAFLEKAYEILRHLAAA

SEQ ID NO: 5 - protein, 123 aa, Artificial Sequence
  boskar_4
AALAAALAEIYKGLAEYQARLKSLEGISPELGPALDALRLDMADFATTMAQAMEEGLDSLPQSFLLKALEQIRKIQADAAALREKLAATYKGNDRAAAAVEIAAQLEAFLEKAYQILRHLAAA

SEQ ID NO: 6 - protein, 118 aa, Artificial Sequence
  moevan
MEAAAAARDESAYLKLQEQMRKIDADAAALSETRTIEELDTFKLDVADFVTTVVQLAEELEHRFGRNRRGRTEIYKIVKEVDRKLLDLTDAVLAKEKKGEDILNMVAEIKALLINIYK

SEQ ID NO: 7 - protein, 115 aa, Artificial Sequence
  moevan_es1.1
MEAAAAARDESAYLKLQEQMRKIDADAAALVQTRTIEELETFKLDVADFITTVVQLAEELIHRLLDRGRTEIYKIVKEVERKLKELTEAVKSKPSGEDILKMVAEIKALLINIYK

SEQ ID NO: 8 - protein, 115 aa, Artificial Sequence
  moevan_es1.2
MEAAAAARDESAYLKLQEQMRKIDADAAALVQTRTIEELETFKLDVADFITTVVQLAEELIHRNTSRGRTEIYKIVKEVERKLKELTEAVKESLNGEDILKMVAEIKALLINIYK

SEQ ID NO: 9 - protein, 115 aa, Artificial Sequence
  moevan_es1.3
MEAAAAARDESAYLKLQEQMRKIDADAAALVQTRTIEELETFKLDVADFITTVVQLAEELIHRKNSRGRTEIYKIVKEVERKLKELTEAVKKGSNGEDILKMVAEIKALLINIYK

SEQ ID NO: 10 - protein, 118 aa, Artificial Sequence
  moevan_ea1.1
MEAAAAARDEKAYIKLQEQMRKIDRDAAALSETRTIEELDKFKLDVADFVSTVVSLAEELEHRFGRNRRGRTEIYKIVKEVDRKLLDLTDAVLAKEKKGEDILNMVAEIKALLINIYK

SEQ ID NO: 11 - protein, 118 aa, Artificial Sequence
  moevan_ea1.2
MEAAAAARDEKAYRKLQEQMRKIDQDAAALSETRTIEELDKFKLDVADFVETVVELAEELEHRFGRNRRGRTEIYKIVKEVDRKLLDLTDAVLAKEKKGEDILNMVAEIKALLINIYK

SEQ ID NO: 12 - protein, 118 aa, Artificial Sequence
  moevan_ea1.3
MEAAAAARDEKAYIKLQEQMRKIDEDAAALSETRTIEELDKFKLDVADFVITVVNLAEELEHRFGRNRRGRTEIYKIVKEVDRKLLDLTDAVLAKEKKGEDILNMVAEIKALLINIYK

SEQ ID NO: 13 - protein, 118 aa, Artificial Sequence
  moevan_ea1.4
MEAAAAARDEKAYWKLQEQMRKIDEDAAALSETRTIEELDKFKLDVADFVETVVRLAEELEHRFGRNRRGRTEIYKIVKEVDRKLLDLTDAVLAKEKKGEDILNMVAEIKALLINIYK

SEQ ID NO: 14 - protein, 154 aa, Artificial Sequence
  sohair
MTSDYIIEQIQRKQEEARLKVEEMERKLEAVKEASKRGVSSDQLLNLILDLADIITTLIQIIEESNEAIKELIKNQKGPTSDYIIEQIQRDQEEARKKVEEAEERLERVKEASKRGVSSDQLLDLIRELAEIIEELIRIIRRSNEAIKELIKNQ

SEQ ID NO: 15 - protein, 154 aa, Artificial Sequence
  sohair_esa1.1
MTSDYIIEQIQRKQELARLKVEEQERKLEEVKEASKRGVSSDQLLQLIQDLADIITTLILIIEESNEAIKELIKNQKGPTSDYIIEQIQRKQELARLKVEEQERKLEEVKEASKRGVSSDQLLQLIQDLADIITTLILIIEESNEAIKELIKNQ

SEQ ID NO: 16 - protein, 154 aa, Artificial Sequence
  sohair_esa1.2
MTSDYIIEQIQRKQEKARLKVEEQERKLEEVKEASKRGVSSDQLLKLIIDLADIITTLIEIIEESNEAIKELIKNQKGPTSDYIIEQIQRKQEKARLKVEEQERKLEEVKEASKRGVSSDQLLKLIIDLADIITTLIEIIEESNEAIKELIKNQ

SEQ ID NO: 17 - protein, 154 aa, Artificial Sequence
  sohair_esa1.3
MTSDYIIEQIQRKQEWARLKVEEQERKLEEVKEASKRGVSSDQLLKLIIDLADIITTLIAIIEESNEAIKELIKNQKGPTSDYIIEQIQRKQEWARLKVEEQERKLEEVKEASKRGVSSDQLLKLIIDLADIITTLIAIIEESNEAIKELIKNQ

SEQ ID NO: 18 - protein, 76 aa, Artificial Sequence
  disohair_1
MTSDYIIEQIQRKQEEARLKVEEMERKLEEVKEASKRGVSSDQLLNLILDLADIITTLIQIIEESNEAIKELIKNQ

SEQ ID NO: 19 - protein, 76 aa, Artificial Sequence
  disohair_2
MTSDYIIEQIQRKQEEARLKVEEQERKLEAVKEASKRGVSSDQLLNLILDLADIITTLIQIIEESNEAIKELIKNQ

SEQ ID NO: 20 - protein, 118 aa, Artificial Sequence
  moevan_ea2.5
MEAAAAARDEKAYRKLQEQMRKIDRDAAALSETRTIEELDRFKLDVADFVQTVVELAEELEHRFGRNRRGRTEIYKIVKEVDRKLLDLTDAVLAKEKKGEDILNMVAEIKALLINIYK

SEQ ID NO: 21 - protein, 118 aa, Artificial Sequence
  moevan_ea2.6
MEAAAAARDEKAYWKLQEQMRKIDEDAAALSETRTIEELDKFKLDVADFVETVVNLAEELEHRFGRNRRGRTEIYKIVKEVDRKLLDLTDAVLAKEKKGEDILNMVAEIKALLINIYK

SEQ ID NO: 22 - protein, 118 aa, Artificial Sequence
  moevan_ea2.7
MEAAAAARDEKAYIKLQEQMRKIDQDAAALSETRTIEELDKFKLDVADFVETVVRLAEELEHRFGRNRRGRTEIYKIVKEVDRKLLDLTDAVLAKEKKGEDILNMVAEIKALLINIYK

SEQ ID NO: 23 - protein, 154 aa, Artificial Sequence
  sohair_esa2.4
MTSDYIIEQIQRKQEIARQKVEEQERKLEEVKEASKRGVSSDQLLKLILDLADIITTLIHIIEESNEAIKELIKNQKGPTSDYIIEQIQRKQEIARQKVEEQERKLEEVKEASKRGVSSDQLLKLILDLADIITTLIHIIEESNEAIKELIKNQ

SEQ ID NO: 24 - protein, 154 aa, Artificial Sequence
  sohair_esa2.5
MTSDYIIEQIQRKQEEARYKVEEQERKLEEVKEASKRGVSSDQLLILIWDLADIITTLIYIIEESNEAIKELIKNQKGPTSDYIIEQIQRKQEEARYKVEEQERKLEEVKEASKRGVSSDQLLILIWDLADIITTLIYIIEESNEAIKELIKNQ

SEQ ID NO: 25 - protein, 154 aa, Artificial Sequence
  sohair_esa2.6
MTSDYIIEQIQRKQEIARWKVEEQERKLEEVKEASKRGVSSDQLLKLIWDLADIITTLIEIIEESNEAIKELIKNQKGPTSDYIIEQIQRKQEIARWKVEEQERKLEEVKEASKRGVSSDQLLKLIWDLADIITTLIEIIEESNEAIKELIKNQ

SEQ ID NO: 26 - protein, 270 aa, Artificial Sequence
  boskar4_t2
AALAAALAEIYKGLAEYQARLKSLEGISPELGPALDALRLDMADFATTMAQAMEEGLDSLPQSFLLKALEQIRKIQADAAALREKLAATYKGNDRAAAAVEIAAQLEAFLEKAYQILRHLAAAGGGGSSGGGGSSGGGGSSGGGGSSAALAAALAEIYKGLAEYQARLKSLEGISPELGPALDALRLDMADFATTMAQAMEEGLDSLPQSFLLKALEQIRKIQADAAALREKLAATYKGNDRAAAAVEIAAQLEAFLEKAYQILRHLAAA

SEQ ID NO: 27 - protein, 252 aa, Artificial Sequence
  boskar4_st2
AALAAALAEIYKGLAEYQARLKSLEGISPELGPALDALRLDMADFATTMAQAMEEGLDSLPQSFLLKALEQIRKIQADAAALREKLAATYKGNDRAAAAVEIAAQLEAFLEKAYQILRHLAAAGGGGSSAALAAALAEIYKGLAEYQARLKSLEGISPELGPALDALRLDMADFATTMAQAMEEGLDSLPQSFLLKALEQIRKIQADAAALREKLAATYKGNDRAAAAVEIAAQLEAFLEKAYQILRHLAAA

SEQ ID NO: 28 - protein, 131 aa, Artificial Sequence
  boskar4_15rl
AALAAALAEIYKGLAEYQARLKSLEGISPELGPALDALRLDMADFATTMAQAMEGGGGSGGGGSGGGGSQSFLLKALEQIRKIQADAAALREKLAATYKGNDRAAAAVEIAAQLEAFLEKAYQILRHLAAA

SEQ ID NO: 29 - protein, 258 aa, Artificial Sequence
  moevan_t2
EAAAAARDESAYLKLQEQMRKIDADAAALSETRTIEELDTFKLDVADFVTTVVQLAEELEHRFGRNRRGRTEIYKIVKEVDRKLLDLTDAVLAKEKKGEDILNMVAEIKALLINIYKGGGGSSGGGGSSGGGGSSGGGGSSEAAAAARDESAYLKLQEQMRKIDADAAALSETRTIEELDTFKLDVADFVTTVVQLAEELEHRFGRNRRGRTEIYKIVKEVDRKLLDLTDAVLAKEKKGEDILNMVAEIKALLINIYK

SEQ ID NO: 30 - protein, 240 aa, Artificial Sequence
  moevan_st2
EAAAAARDESAYLKLQEQMRKIDADAAALSETRTIEELDTFKLDVADFVTTVVQLAEELEHRFGRNRRGRTEIYKIVKEVDRKLLDLTDAVLAKEKKGEDILNMVAEIKALLINIYKGGGGSSEAAAAARDESAYLKLQEQMRKIDADAAALSETRTIEELDTFKLDVADFVTTVVQLAEELEHRFGRNRRGRTEIYKIVKEVDRKLLDLTDAVLAKEKKGEDILNMVAEIKALLINIYK

SEQ ID NO: 31 - protein, 164 aa, Artificial Sequence
  sohair2_15rl
MTSDYIIEQIQRKQEEARLKVEEQERKLEAVKEASKRGVSSDQLLNLILDLADIITTLIQIIEESNEAIKELIKNQGGGGSGGGGSGGGGSDYIIEQIQRKQEEARLKVEEQERKLEAVKEASKRGVSSDQLLNLILDLADIITTLIQIIEESNEAIKELIKNQ

SEQ ID NO: 32 - protein, 63 aa, Artificial Sequence
  bika1
SKEVLEQSLFLKLDEQVRKLLADIHAIKIDRITGNMDKQKLDTAYLDVADIETTLYQLIEVSH

SEQ ID NO: 33 - protein, 63 aa, Artificial Sequence
  bika2
SKEVLEQSLFLKLDEQVRKLLADIHAIKIDRITGNMDKQKLDTLYLDVADIETTLYQLIEVSH

(19) United States
(12) Patent Application Publication
(10) Pub. No.: US 2023/0227520 A1
(43) Pub. Date: Jul. 20, 2023

(54) NOVEL G-CSF MIMICS AND THEIR APPLICATIONS

(71) Applicants: Max-Planck-Gesellschaft zur Förderung der Wissenschaften e.V., München (DE); Eberhard Karls Universität Tübingen, Tübingen (DE)

(72) Inventors: Mohammad ELGAMACY, Tübingen (DE); Birte HERNANDEZ ALVAREZ, Dettenhausen (DE); Yulia SKOKOWA, Kusterdingen (DE)

(73) Assignees: Max-Planck-Gesellschaft zur Förderung der Wissenschaften e.V., München (DE); Eberhard Karls Universität Tübingen, Tübingen (DE)

(21) Appl. No.: 17/757,550

(22) PCT Filed: Dec. 17, 2020
(86) PCT No.: PCT/EP2020/086843

§ 371 Date: Jun. 16, 2022

(30) Foreign Application Priority Data
Dec. 17, 2019 (EP) 19217185.8

(51) Int. Cl. C07K 14/535; C12N 5/00; A61P 7/00; A61K 38/18

(52) U.S. Cl. CPC C07K 14/535; C12N 5/0018; A61P 7/00; A61K 38/18

(57) ABSTRACT

The present invention relates to a protein having G-CSF-like activity comprising a) one or two polypeptide chains; b) a bundle of four α-helices; and c) two or three amino acid linkers that connect contiguous bundle-forming α-helices that are located on the same polypeptide chain, wherein each amino acid linker has a length between 2 and 20 amino acids. The invention also provides for a polynucleotide and a vector encoding the protein of the invention, host cells comprising said polynucleotide, a method for producing the protein of the invention and a pharmaceutical composition comprising the protein of the invention. The invention further relates to uses of the proteins of the invention as a research reagent and the use of the protein and/or pharmaceutical composition comprising the same as a medicament, e.g., for use in increasing stem cell production, for use in inducing hematopoiesis and/or for use in mobilizing hematopoietic stem cells.

The present application is a national phase application under 35 U.S.C. § 371 of International Application No. PCT/EP2020/086843, filed Dec. 17, 2020, the entire contents of which are hereby incorporated by reference. International Application No. PCT/EP2020/086843 claims the priority benefit of European Application No. 19217185.8, filed Dec. 17, 2019.

This application contains a Sequence Listing which has been submitted electronically in ASCII format and is hereby incorporated by reference in its entirety. The ASCII copy, created on Oct. 21, 2022, is named VOSSP0130US_ST25_updated.txt and is 41,874 bytes in size.

The present invention relates to novel proteins with G-CSF-like activity, pharmaceutical compositions comprising a protein of the invention and polynucleotides encoding the proteins of the invention. Further, a host cell comprising and expressing a polynucleotide of the invention, methods for producing a protein of the invention and uses of a protein according to the invention as research reagent are provided. The invention also relates to the proteins of the invention or pharmaceutical compositions of the invention for use as a medicament.

Protein therapeutics have been the fastest growing class of approved drugs during the past decade [1]. While small molecule drugs are often restricted to binding to hydrophobic pockets on their targets, proteins possess larger interaction surface areas, which render their interactions more specific and allow addressing previously undruggable targets. Moreover, protein molecules, spanning antibodies, enzymes and receptor modifiers [1], have provided molecular platforms that can be readily reengineered for therapeutic purposes starting from their natural templates [2].

Cytokines serve as a major class of clinically relevant proteins. Upon understanding their central homeostatic roles it has become possible to develop several cytokine and anti-cytokine therapies, which are now approved and widely used in clinical settings [3]. Cytokines constitute a loose category of small- to medium-sized peptides and glycoproteins that are produced by different cell types and play important roles in mediating autocrine, paracrine and endocrine signaling in a wide range of cellular responses. These molecules act through binding to specific membrane receptors and induce dimerization or activation of receptor subunits, which can then activate downstream second messenger cell signaling pathways, such as JAK/STAT, Akt or Erk pathways [4]. When used in clinical settings, cytokines are frequently used as natural templates or with only minor sequence alterations. Yet, Silva et al. recently described a de novo computational approach for designing proteins that recapitulate the binding sites of the natural cytokines IL-2 and IL15, respectively, but are otherwise unrelated in topology or amino acid sequence with the natural cytokines [28].

Colony stimulating factors (CSF) are glycoproteins that constitute a subclass of cytokines essential for the differentiation of several leukocyte types from bone marrow cells. The granulocyte colony-stimulating factor (G-CSF or CSF3) is a CSF that stimulates the proliferation and differentiation of neutrophil progenitors in the bone marrow and their release into the blood stream. G-CSF has attracted special attention due to its potency as an inflammatory response enhancing and host immunity enhancing agent through neutrophil stimulation in neutropenic cases. The administration of G-CSF is usually well tolerated and its cell proliferation response resembles an infection-evoked response [5]. Filgrastim, a recombinant, unglycosylated human G-CSF variant produced in E. coli, was approved and has been used since 1991 in the treatment of neutropenia to mobilize hematopoietic progenitor cells following myelosuppressive chemotherapy, bone marrow transplantation, or radiotherapy [6]. This attracted many research efforts aiming to enhance its biological activity and pharmacological specificity, improve its stability, and lower its production costs.

The granulocyte colony-stimulating factor receptor (G-CSF-R) also known as CD114 (Cluster of Differentiation 114) is a protein that, in humans, is encoded by the CSF3R gene. G-CSF-R is a cell-surface receptor for G-CSF and belongs to a family of cytokine receptors known as the hematopoietin receptor family. G-CSF-R is, amongst others, present on precursor cells in the bone marrow, and, in response to G-CSF stimulation, initiates cell proliferation and differentiation into mature neutrophilic granulocytes and other cell types. The G-CSF-R is a transmembrane receptor that consists of an extracellular ligand-binding portion, a transmembrane domain, and the cytoplasmic portion that is responsible for signal transduction. G-CSF-R ligand-binding is associated with dimerization of the receptor and signal transduction through proteins including Jak, Lyn, STAT, and Erk1/2.

The structure of human G-CSF comprises a bundle of four nearly parallel and antiparallel α-helices. Helix A consists of about 27 amino acids (residues 11-37), helix B consists of about 17 amino acids (residues 74-90), helix C consists of about 22 amino acids (residues 101-122), and helix D consists of about 30 amino acids (residues 143-171). In addition, a crossover region that contains a 7-residue α-helix (residue 48-54), helix E, along a loop that connects helix A to helix B is comprised in the structure of G-CSF. The four main α-helices A-D are arranged in an up-up-down-down topology, with two long bundle-spanning linkers connecting α-helices A and B, as well as α-helices C and D. Both the length of the protein and the structural features of G-CSF place it within the long-chain cytokine subfamily. G-CSF has five cysteine residues, with four of these cysteines forming disulfide bonds (Cys36-Cys42 and Cys64-Cys74). G-CSF expressed in mammalian cells further contains an O-linked glycan on residue threonine 133, but glycosylation is not required for biological activity as demonstrated by filgrastim, which is expressed in bacterial cells and is not glycosylated.

It has been shown that the G-CSF long loops display fast motions with fairly low average S² order parameter of 0.57 and a very fast local internal correlation time (τ_{c}) of 0.42 ns. The A-B loop is, however, more structured than the C-D loop, owing to the two disulfide bonds tethering it to helices A and B, in addition to the presence of the interrupting helix E (see FIG. 1) [12]. Nonetheless, these disulfide bonds, along with an extra free cysteine (C17), have been shown to result in persistent aggregates, and thus affect the activity shelf-life of filgrastim [25]. These loops also often comprise spans of missing electron densities in several crystallographic structures of human G-CSF.

The short circulation half-life of filgrastim of about 3.5 hours [7] encouraged several attempts to engineer more stable, long-acting filgrastim biobetters. Numerous research studies investigated PEGylation as a means to generate more soluble and stable forms. This strategy faced considerable challenges during the development of different PEGylation approaches, including difficulties related to molecular weight heterogeneity, activity interference and product consistency. Nevertheless, different PEGylated forms have successfully gained approval while others are still undergoing clinical trials [8]. Another approach employed two successive reengineering cycles of glycine-to-alanine mutagenesis and yielded mutants with folding free energy change (ΔΔG) of approximately −3 kcal/mol before drastically reducing the activity [9]. Most recently, a polypeptide circularization strategy with and without sequence optimization of the circularization loop has yielded melting temperature (Tₘ) enhancements of 4.2° C. and 12.9° C., respectively [10].

WO 94/017185 discloses methods for the preparation of G-CSF mutant variants. WO 94/017185 further speculates that deletions in the external loops of G-CSF may result in increased protein half-life. However, no experimental examples of such deletion mutants are provided in WO 94/017185.

WO 2006/128176 discloses fusion proteins comprising G-CSF. As in the case of WO 94/017185, WO 2006/128176 merely speculates that deletions in the external loops may increase half-life of the fusion protein.

Bazan et al. (Immunology Today, 1990, 11(10), p. 350-354) is a review article directed to cytokines in general. In one paragraph, Bazan et al. speculate that cytokine analogs may be computationally designed. However, no teaching how to obtain such variants is provided.

Kuga et al. (Biochemical and Biophysical Research Communications, 1989, 159(1), p. 103-111) discloses various mutant variants of G-CSF. Of the obtained mutant variants, only the ones with mutations or deletions in the unstructured N-terminal part of G-CSF retained activity.

Like most other therapeutic proteins, G-CSF has been clinically deployed as is, or with few engineered modifications of its natural template. The challenges linked to use of the natural G-CSF protein are evidenced by the low recombinant production yield, the low solubility and the low stability of filgrastim [10, 11]. It is of note that filgrastim can only be produced at low yields from bacterial expression hosts as it is expressed in inclusion bodies and has to be refolded following a laborious refolding strategy.

Accordingly, there is a need for G-CSF-like proteins with improved properties for the use in therapeutic and research applications. In particular, there is a need to provide G-CSF-like proteins that are more stable, protease resistant and/or can be easier produced (e.g. in bacterial hosts), preferably at a higher yield and without cumbersome refolding strategies.

The above technical problem is solved by the present invention as defined in the claims and as described herein below.

The inventors developed a sophisticated protein design approach (see Example 1) to provide new non-naturally occurring proteins with G-CSF-like activity. In contrast to previous engineering approaches, said computer-assisted design approach involves structural re-scaffolding of the G-CSF receptor binding sites to provide smaller and topologically simpler proteins that possess different folds and sequences from natural G-CSF, while being pharmacologically active. Specifically, the inventors preserved the steric and electrostatic features of the G-CSF receptor binding site as a design constraint, while diversifying the protein scaffolding. The inventors demonstrate that this protein scaffolding refactoring strategy surprisingly generates molecules that exhibit G-CSF-like activity, but with different topologies, biophysical properties, different folds and only minimal full-length sequence homology to natural G-CSF. In particular, the inventors could demonstrate in the appended examples that the new G-CSF-like proteins of the invention show increased thermal stability and can be produced as soluble and folded proteins without the formation of inclusion bodies that would require refolding. Moreover, most of the provided proteins show a massively increased resistance to the protease neutrophil elastase, which is known to degrade G-CSF in vivo [18, 19]. Providing a smaller and more stable G-CSF-like protein that is easier to purify at higher yield can improve G-CSF treatment, which is widely used in a number of medical implications. It is envisaged that the increased stability and/or protease resistance of the proteins of the invention improves shelf-life and dosage form properties (e.g. decrease protein precipitation and possess longer room-temperature shelf-life. In addition, it is envisaged that the proteins of the inventions possess higher in vivo duration of action in comparison to wild type G-CSF and can, thus, e.g., prolong the re-administration intervals.

The invention relates to the following aspects:
1. A protein comprising:
   a) one or two polypeptide chains;
   b) a bundle of four α-helices; and
   c) two or three amino acid linkers that connect contiguous bundle-forming α-helices that are located on the same polypeptide chain, wherein each amino acid linker has a length between 2 and 20 amino acids;
wherein the protein has G-CSF-like activity.
2. The protein according to aspect 1, wherein the protein comprises one or more G-CSF receptor binding sites and/or wherein the protein has a melting temperature (Tₘ) of at least 74° C.
3. The protein according to any one of aspects 1 to 2, wherein the G-CSF-like activity comprises at least one, preferably at least two, more preferably at least three, most preferably all of the following activities:
   (i) induction of granulocytic differentiation of HSPCs;
   (ii) induction of the formation of myeloid colony-forming units from HSPCs;
   (iii) induction of the proliferation of NFS-60 cells; and/or
   (iv) activation of the downstream signaling pathways MAPK/ERK and/or JAK/STAT.
4. The protein according to any one of aspects 1 to 3, wherein the protein induces the proliferation and/or differentiation of cells comprising one or more G-CSF receptor on the cell surface.
5. The protein according to aspect 4, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.
6. The protein according to any one of aspects 1 to 5, wherein the calculated contact order number of said protein is lower than the calculated contact order number of human G-CSF (SEQ ID NO:1).
7 The protein according to any one of aspects 1 to 6, wherein the protein has a molecular mass between 13 and 18 kDa.
8. The protein according to any one of aspects 1 to 7, wherein the protein comprises no disulfide bonds.
9. The protein according to any one of aspects 1 to 8, wherein the protein is not glycosylated.
10. The protein according to any one of aspects 1 to 9, wherein the α-helices that form the bundle of four α-helices are located on a single polypeptide chain.
11. The protein according to aspect 10, wherein the single polypeptide chain comprises a four-helix bundle arrangement.
12. The protein according to aspect 11, wherein the four-helix bundle arrangement has an up-down-up-down topology.
13. The protein according to any one of aspects 10 to 12, wherein the single polypeptide chain comprises an amino acid sequence having at least 60%, 70%, 80%, 90% amino acid sequence identity with an amino acid sequence selected from the group consisting of: SEQ ID NO:5, SEQ ID NO: 4, SEQ ID NO:3, SEQ ID NO:2, SEQ ID NO:6 and SEQ ID NO:14.
14. The protein according to any one of aspects 10 to 12, wherein the single polypeptide chain comprises an amino acid sequence selected from the group consisting of: SEQ ID NO:5, SEQ ID NO: 4, SEQ ID NO:3, SEQ ID NO:2, SEQ ID NO:6 and SEQ ID NO:14.
15. The protein according to any one of aspects 1 to 9, wherein the α-helices that form the bundle of four α-helices are located on two separate polypeptide chains.
16. The protein according to aspect 15, wherein each of the two polypeptide chains contributes two α-helices to the bundle of four α-helices.
17. The protein according to any one of aspects 15 to 16, wherein each of the two polypeptide chains comprises a helical-hairpin motif.
18. The protein according to any one of aspects 15 to 17, wherein the two polypeptide chains form a dimer.
19. The protein according to any one of aspects 15 to 18, wherein both polypeptide chains comprise an amino acid sequence having at least 60%, 70%, 80%, 90% amino acid sequence identity with an amino acid sequence selected from the group consisting of: SEQ ID NO:19 and SEQ ID NO:18.
20. The protein according to any one of aspects 15 to 18, wherein both polypeptide chains comprise an amino acid sequence selected from the group consisting of: SEQ ID NO:19 and SEQ ID NO:18.
21. The protein according to any one of aspects 1 to 20, wherein the spatial orientation and molecular interaction features of at least two, at least three, at least four, at least five, at least six, at least seven of the amino acid residues Lysine 16, Glutamate 19, Glutamine 20, Arginine 22, Lysine 23, Aspartate 27, Asparagine 109, and Aspartate 112 of human G-CSF (SEQ ID NO:1) are preserved.
22. A protein comprising or consisting of an amino acid sequence having at least 60%, 70%, 80%, 90%, 95%, 96%, 97%, 98%, 99%, or 100% amino acid sequence identity with the amino acid sequence of SEQ ID NO:5, wherein the protein has G-CSF-like activity.
23. The protein according to aspect 22, wherein the protein comprises: a) a bundle of four α-helices; and b) three amino acid linkers that connect contiguous bundle-forming α-helices, wherein each amino acid linker has a length between 2 and 20 amino acids.
24. The protein according to any one of aspects 22 to 23, wherein the protein comprises one or more G-CSF receptor binding sites.
25. The protein according to any one of aspects 22 to 24, wherein the G-CSF-like activity comprises at least one, preferably at least two, more preferably at least three, most preferably all of the following activities:
   (i) induction of granulocytic differentiation of HSPCs;
   (ii) induction of the formation of myeloid colony-forming units from HSPCs;
   (iii) induction of the proliferation of NFS-60 cells; and/or
   (iv) activation of the downstream signaling pathways MAPK/ERK and/or JAK/STAT.
26. The protein according to any one of aspects 22 to 25, wherein the protein induces the proliferation and/or differentiation of cells comprising one or more G-CSF receptor on the cell surface.
27. The protein according to aspect 26, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.
28. The protein according to any one of aspects 22 to 27, wherein the calculated contact order number of said protein is lower than the calculated contact order number of human G-CSF (SEQ ID NO:1).
29. The protein according to any one of aspects 22 to 28, wherein the protein has a molecular mass between 12 and 15 kDa.
30. The protein according to any one of aspects 22 to 29, wherein the protein comprises no disulfide bonds.
31. The protein according to any one of aspects 22 to 30, wherein the protein is not glycosylated.
32. A protein comprising or consisting of an amino acid sequence having at least 60%, 70%, 80%, 90%, 95%, 96%, 97%, 98%, 99%, or 100% amino acid sequence identity with the amino acid sequence of SEQ ID NO:6, wherein the protein has G-CSF-like activity.
33. The protein according to aspect 32, wherein the protein comprises: a) a bundle of four α-helices; and b) three amino acid linkers that connect contiguous bundle-forming α-helices, wherein each amino acid linker has a length between 2 and 20 amino acids.
34. The protein according to any one of aspects 32 to 33, wherein the protein comprises one or more G-CSF receptor binding sites.
35. The protein according to any one of aspects 32 to 34, wherein the G-CSF-like activity comprises at least one, preferably at least two, more preferably at least three, most preferably all of the following activities:
   (i) induction of granulocytic differentiation of HSPCs;
   (ii) induction of the formation of myeloid colony-forming units from HSPCs;
   (iii) induction of the proliferation of NFS-60 cells; and/or
   (iv) activation of the downstream signaling pathways MAPK/ERK and/or JAK/STAT.
36. The protein according to any one of aspects 32 to 35, wherein the protein induces the proliferation and/or differentiation of cells comprising one or more G-CSF receptor on the cell surface.
37. The protein according to aspect 36, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.
38. The protein according to any one of aspects 32 to 37, wherein the calculated contact order number of said protein is lower than the calculated contact order number of human G-CSF (SEQ ID NO:1).
39. The protein according to any one of aspects 32 to 38, wherein the protein has a molecular mass between 12 and 15 kDa.
40. The protein according to any one of aspects 32 to 39, wherein the protein comprises no disulfide bonds.
41. The protein according to any one of aspects 32 to 40, wherein the protein is not glycosylated.
42. A protein comprising or consisting of an amino acid sequence having at least 60%, 70%, 80%, 90%, 95%, 96%, 97%, 98%, 99%, or 100% amino acid sequence identity with the amino acid sequence of SEQ ID NO:14, wherein the protein has G-CSF-like activity.
43. The protein according to aspect 42, wherein the protein comprises: a) a bundle of four α-helices; and b) three amino acid linkers that connect contiguous bundle-forming α-helices, wherein each amino acid linker has a length between 2 and 20 amino acids.
44. The protein according to any one of aspects 42 to 43, wherein the protein comprises one or more G-CSF receptor binding sites.
45. The protein according to any one of aspects 42 to 44, wherein the G-CSF-like activity comprises at least one, preferably at least two, more preferably at least three, most preferably all of the following activities:
   (i) induction of granulocytic differentiation of HSPCs;
   (ii) induction of the formation of myeloid colony-forming units from HSPCs;
   (iii) induction of the proliferation of NFS-60 cells; and/or
   (iv) activation of the downstream signaling pathways MAPK/ERK and/or JAK/STAT.
46. The protein according to any one of aspects 42 to 45, wherein the protein induces the proliferation and/or differentiation of cells comprising one or more G-CSF receptor on the cell surface.
47. The protein according to aspect 46, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.
48. The protein according to any one of aspects 42 to 47, wherein the calculated contact order number of said protein is lower than the calculated contact order number of human G-CSF (SEQ ID NO:1).
49. The protein according to any one of aspects 42 to 48, wherein the protein has a molecular mass between 16 and 18 kDa.
50. The protein according to any one of aspects 42 to 49, wherein the protein comprises no disulfide bonds.
51. The protein according to any one of aspects 42 to 50, wherein the protein is not glycosylated.
52. A protein comprising an amino acid sequence having at least 60%, 70%, 80%, 90%, 95%, 96%, 97%, 98%, 99%, or 100% amino acid sequence identity with the amino acid sequence of SEQ ID NO:19, wherein the protein has G-CSF-like activity.
53. The protein according to aspect 52, wherein the protein comprises: a) two polypeptide chains; (b) a bundle of four α-helices; and c) two amino acid linkers that connect contiguous bundle-forming α-helices that are located on the same polypeptide chain, wherein each amino acid linker has a length between 2 and 20 amino acids, preferably wherein the two polypeptide chains of the protein comprise identical amino acid sequences.
54. The protein according to any one of aspects 52 to 53, wherein the protein comprises one or more G-CSF receptor binding sites.
55. The protein according to any one of aspects 52 to 54, wherein the G-CSF-like activity comprises at least one, preferably at least two, more preferably at least three, most preferably all of the following activities:
   (i) induction of granulocytic differentiation of HSPCs;
   (ii) induction of the formation of myeloid colony-forming units from HSPCs;
   (iii) induction of the proliferation of NFS-60 cells; and/or
   (iv) activation of the downstream signaling pathways MAPK/ERK and/or JAK/STAT.
56. The protein according to any one of aspects 52 to 55, wherein the protein induces the proliferation and/or differentiation of cells comprising one or more G-CSF receptor on the cell surface.
57. The protein according to aspect 56, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.
58. The protein according to any one of aspects 52 to 57, wherein the calculated contact order number of said protein is lower than the calculated contact order number of human G-CSF (SEQ ID NO:1).
59. The protein according to any one of aspects 52 to 58, wherein the protein has a molecular mass between 16 and 18 kDa.
60. The protein according to any one of aspects 52 to 59, wherein the protein comprises no disulfide bonds.
61. The protein according to any one of aspects 52 to 60, wherein the protein is not glycosylated.
62. A polynucleotide encoding the protein according to any one of aspects 1 to 61.
63. The polynucleotide according to aspect 62, wherein the polynucleotide is operably linked to at least one promoter capable of directing expression in a cell.
64. A vector comprising the polynucleotide according to any one of aspects 62 to 63.
65. A host cell genetically transformed with the polynucleotide of any one of aspects 62 to 63 or the vector according to aspect 64, preferably wherein the host cell expresses the protein according to the invention.
66. A method for producing a protein according to any one of aspects 1 to 61, the method comprising the steps of: i) cultivating the host cell according to aspect 65; and (ii) recovering the protein of the invention from the cell culture and/or host cells.
67. A pharmaceutical composition comprising the protein according to any one of aspects 1 to 61, the polynucleotide according to any one of aspects 62 to 63, the vector according to aspect 64, and/or the cell according to aspect 65.
68. The pharmaceutical composition according to aspect 67, wherein said pharmaceutical composition is administered in combination with myelosuppressive agent and/or an immunostimulant.
69. The pharmaceutical composition according to aspect 68, wherein the myelosuppressive agent is a chemotherapeutic agent and/or an antiviral agent.
70. The protein according to any one of aspects 1 to 61 or the pharmaceutical composition according to any one of aspects 67 to 69 for use as a medicament.
71. The protein according to any one of aspects 1 to 61 or the pharmaceutical composition according to any one of aspects 67 to 69 for use in increasing stem cell production.
72. The protein according to any one of aspects 1 to 61 or the pharmaceutical composition according to any one of aspects 67 to 69 for use in inducing hematopoiesis.
73. The protein according to any one of aspects 1 to 61 or the pharmaceutical composition according to any one of aspects 67 to 69 for use in increasing the number of granulocytes.
74. The protein according to any one of aspects 1 to 61 or the pharmaceutical composition according to any one of aspects 67 to 69 for use in accelerating neutrophil recovery following hematopoietic stem cell transplantation.
75. The protein according to any one of aspects 1 to 61 or the pharmaceutical composition according to any one of aspects 67 to 69 for use in preventing, treating, and/or alleviating myelosuppression resulting from a chemotherapy and/or radiotherapy.
76. The protein according to any one of aspects 1 to 61 or the pharmaceutical composition according to any one of aspects 67 to 69 for use in treating a subject having neutropenia.
77. The protein according to any one of aspects 1 to 61 or the pharmaceutical composition according to any one of aspects 67 to 69 for use in treating neurological disorders.
78. The protein according to any one of aspects 1 to 61 or the pharmaceutical composition according to any one of aspects 67 to 69 for use in stem cell mobilization.
79. The protein or pharmaceutical composition according to aspect 78, wherein the protein according to the invention is administered in combination with at least one additional stem cell mobilizing agent.
80. Use of the protein according to any one of aspects 1 to 61 as an additive in a cell culture.
81. Use of the protein according to aspect 80, wherein the protein stimulates the proliferation and/or differentiation of cells in a cell culture.
82. A method for proliferating and/or differentiating cells in a cell culture comprising contacting said cells with the protein according to any one of aspects 1 to 61.

Accordingly, in one aspect, the present invention relates to a protein comprising: a) one or two polypeptide chains; b) a bundle of four α-helices; and c) two or three amino acid linkers that connect contiguous bundle-forming α-helices that are located on the same polypeptide chain, wherein each amino acid linker has a length between 2 and 20 amino acids; wherein the protein has G-CSF-like activity. The G-CSF-like protein according to the invention preferably comprises at least one G-CSF receptor (G-CSF-R) binding site. Further, the G-CSF-like protein according to the invention preferably has a melting temperature (Tₘ) of at least 74° C.

That is, the invention is based, at least in part, on the unexpected discovery that proteins with very low sequence identity with G-CSF are able to exhibit G-CSF-like activity. Direct comparison of sequence identities of the G-CSF-like protein variants of the present invention with human G-CSF shows that the protein variants named Boskar_1 (SEQ ID NO:2), Boskar_2 (SEQ ID NO:3), Boskar_3 (SEQ ID NO:4) and Boskar_4 (SEQ ID NO:5) have a sequence identity with human G-CSF of less than 50% over the whole length of the protein, while the protein variants called Moevan (SEQ ID NO:6), Sohair (SEQ ID NO:14), Disohair_1 (SEQ ID NO:18) and Disohair_2 (SEQ ID NO:19) have even lower sequence identities with human G-CSF over the whole length of the protein (Table 2). Thus, it has to be considered highly surprising that proteins with such low sequence identities compared to G-CSF may carry out similar functions as human G-CSF.

Despite differing greatly in their amino acid sequence, the protein designs of the invention have several unifying features, namely a four-helix bundle arrangement comprising linkers that are significantly shorter than in human G-CSF. In addition, or as a consequence, the protein designs of the invention have high thermal and/or protease stability while carrying out at least one G-CSF-like activity.

The protein according to the invention comprises a bundle of four α-helices and may further comprise one or two polypeptide chains. Accordingly, the four α-helices that form the bundle of four α-helices may be located on a single polypeptide chain comprising all four α-helices, or may be located on two separate polypeptide chains that comprise between one and three α-helices. The latter case is exemplified by the Disohair variants (SEQ ID NO:18-19), which comprise two polypeptide chains comprising two α-helices, respectively. The number of polypeptide chains further determines the number of amino acid linkers between contiguous α-helices. In cases where all four α-helices are located on a single polypeptide chain, the protein according to the invention may comprise three amino acid linkers that connect contiguous α-helices that are located on the same polypeptide chain. In cases where the α-helices are located on two separate polypeptide chains, the protein of the invention may comprise only two amino acid linkers that connect contiguous α-helices that are located on the same polypeptide chain.

A significant structural difference between G-CSF and the protein according to the invention may be seen in the length of the amino acid linkers that connect contiguous α-helices that are located on the same polypeptide chain. In human G-CSF, the amino acid linkers between the four main α-helices A, B, C and D have a length of about 10 to 36 amino acids, while the amino acid linkers of the protein variants of the present invention have a length of 2 to 20 amino acids, preferably between 3 to 7 amino acids. As illustrated in Table 3, the exemplary protein designs have in common that the length of the amino acid linkers between the four main α-helices are between 3 to 7 amino acids in length, i.e. are shorter than 20, preferably 18, preferably 16, preferably 14, preferably 12 and most preferably 10 amino acids. Without being bound to theory, the shorter linkers may presumably contribute to the improved stability of these protein variants in comparison with natural G-CSF. Thus, the G-CSF-like protein according to the present invention may comprise amino acid linkers connecting contiguous α-helices that are located on the same polypeptide chain that have a length between 2 and 20, preferably between 2 and 15, more preferably between 2 and 10, and most preferably between 3 and 7 amino acids.

In a certain embodiment, the present invention relates to a protein comprising: a) one or two polypeptide chains; b) a bundle of four α-helices; and c) two or three amino acid linkers that connect contiguous bundle-forming α-helices that are located on the same polypeptide chain, wherein each amino acid linker has a length between 2 and 15 amino acids; wherein the protein has G-CSF-like activity.

All protein variants disclosed herein have a unifying structural feature, namely the presence of a bundle of four α-helices, wherein the linkers between these α-helices have a length between 2 and 15 amino acids. G-CSF-like proteins comprising such short linkers can only be obtained by protein remodeling and not by conventional protein engineering approaches. Due to these short linkers, the G-CSF-like proteins of the invention have various advantages over G-CSF analogs known in the art, such as higher thermal stability, higher solubility and higher expression levels in bacterial host cells. To this end, it has to be noted that a protein variant comprising a 15 amino acid linker has been demonstrated herein to be biologically active (variant boskar4_15 rl (SEQ ID NO:28) in Table 7).

In contrast, WO 94/17185 speculates about a G-CSF analog wherein the amino acid residues 58-72 in the linker connecting helices A and B are deleted, thereby reducing the length of this linker to 18 amino acid residues (amino acid residues 40-57 according to the numbering in WO 94/17185). Such a variant would comprise linkers between the four main α-helices having a maximal length of 19 amino acid residues (linker between helices C and D). However, further shortening of these linkers is not possible due to the up-up-down-down topology of this variant.

The skilled person is aware of methods to determine structural features of a protein such as α-helices or beta-sheets and/or linker sequences between such structures. The most common methods to determine the three dimensional structure of a protein are X-ray crystallography, NMR spectroscopy and cryo-electron microscopy. These methods may be applied to detect the position and lengths of α-helices in a protein and the amino acids involved in the formation of these α-helices. Further, the methods may be applied to determine the length of amino acid linkers between two contiguous α-helices located on the same polypeptide chain and to identify the amino acids that form these linkers (i.e. the position and length of such linkers in the amino acid sequence), if these linkers are structured. In addition, these methods may be applied to determine the orientation of α-helices towards each other, for example parallel or antiparallel orientation, within a protein. Further biophysical methods that may be applied to determine secondary structures of proteins include circular dichroism (CD) spectroscopy and Fourier-transform infrared (FTIR) spectroscopy.

Alternatively, structural features of proteins such as, for example, the lengths of α-helices and/or amino acid linkers, may be predicted by using computational methods that start from the primary amino acid sequence of a protein. Several computer programs are known in the art that may be applied for the prediction of secondary protein structures. By way of non-limiting example, suitable computer programs include Psipred [29], SPIDER2 [30], PSSPred [https://zhanglab.ccmb.med.umich.edu/PSSpred/], DeepCNF [31] and Coils [32]. One or more computer programs may be used for the prediction of a protein structure. Adaptation of the settings may be required to be able to directly compare the results of the different programs. The computer programs may be used in combination with experimental data to refine the results of the computational prediction.

FIG. 12 shows the agreement between the determined NMR structures for Moevan and Sohair and their respective design models, showing the design models (cartoon representation) structurally aligned against the NMR ensemble (ribbon representation). Moevan showed an ensemble backbone RMSD from the average structure of 1.8 Å, and 2.46 Å from the design (FIG. 12A). Sohair showed an ensemble backbone RMSD from the average structure of 1.78 Å, and 2.85 Å from the design (FIG. 12B). Similar studies have been performed for the variant Boskar_4 (FIG. 17).

More specifically, a preferred prediction program for determining the secondary structure of proteins and to determine the length of amino acid linkers connecting contiguous α-helices in the context of the present invention is Psipred. The program is preferably used with an E-value of 10⁻³, having all other parameters at the default setting.

One of the main limitations of G-CSF in therapeutic or diagnostic applications is its low stability, which results in short circulation half-life and low production levels (involving a cumbersome refolding approach). Without being bound to theory, the low stability of G-CSF and the insolubility in the bacterial expression system is at least to some extent caused by the long linkers that connect the α-helices, particularly the long bundle-spanning linkers between α-helices A and B, as well as α-helices C and D, which make the protein thermally unstable and susceptible for proteolytic lysis.

To overcome this limitation, the inventors pursued computational protein design approaches to obtain smaller and topologically simpler proteins that still possess G-CSF-like activity. This was achieved by preserving the binding site of G-CSF that is required for interacting with the G-CSF receptor G-CSF-R, while the scaffold of the protein was drastically re-engineered in order to obtain proteins with higher stability. An improved thermal stability was exemplary demonstrated for the protein variants Boskar_4 (SEQ ID NO:5), Moevan (SEQ ID NO:6) and Disohair_2 (SEQ ID NO:19) in comparison to G-CSF in Example 3 (FIG. 2 and Table 6).

Thermal stability assays coupled to circular dichroism revealed that G-CSF shows a complete unfolding transition at approximately 330 Kelvin and misfolds upon cooling. The protein variants of the present invention, however, unfolded at significantly higher temperatures or even remained stable at temperatures above 370 Kelvin. In view of the design strategy for the proteins variants of the present invention it is expected that all other designs show a similarly improved thermal stability. Thus, it is plausible that all proteins falling under the structural definition of the invention have a higher thermal stability than G-CSF and thus solve the technical problem of the invention to provide a more stable G-CSF analog.

In addition, Example 4 (FIGS. 3 and 4) documents that the protein variants Boskar_4 (SEQ ID NO:5) and Disohair_2 (SEQ ID NO:19) have a higher resistance against the protease neutrophil elastase. Taken together, the proteins according to the invention are more stable than G-CSF, while maintaining G-CSF-like activity. In view of the above, the protein according to the invention may have a longer circulation half-life when administered to a subject and may thus allow less frequent, and eventually cheaper, dosing regimens.

The inventors also found that the G-CSF-like protein variants of the invention are expressed as soluble proteins in bacterial hosts, such as E. coli, so that cumbersome refolding strategies can be avoided (see Example 2). The purification resulted in much higher yields as achieved by the purification scheme of wild type G-CSF which is expressed in inclusion bodies and involves denaturation and refolding (see FIG. 7 and table 6). Thus, the proteins of the invention can be easier and more efficiently produced.

It has been shown by the inventors that G-CSF precipitates in 1×PBS buffer at concentrations above 4 mg/mL. In contrast, the proteins according to the invention remained soluble at concentrations above 4 mg/mL (Table 6).

Accordingly, in certain embodiments, the invention relates to the G-CSF-like protein according to the invention, wherein the protein remains soluble in an aqueous solution at a protein concentration of at least 5 mg/mL, at least 6 mg/mL, at least 7 mg/mL. at least 8 mL, at least 9 mg/mL, at least 10 mg/mL, at least 11 mg/mL, at least 12 mg/mL, at least 13 mg/mL, at least 14 mg/mL, at least 15 mg/mL, at least 16 mg/mL, at least 17 mg/mL, at least 18 mg/mL, at least 19 mg/mL or at least 20 mg/mL. The skilled person is aware of methods to determine the solubility of a protein in solution. Preferably, the solubility of the protein according to the invention is determined in 1×PBS buffer at 25° C.

As used herein, “solubility” with reference to a protein refers to a protein that is homogenous in an aqueous solution, whereby protein molecules diffuse and do not sediment spontaneously. Hence a soluble protein solution is one in which there is an absence of a visible or discrete particle in a solution containing the protein, such that the particles cannot be easily filtered. Generally, a protein is soluble if there are no visible or discrete particles in the solution. For example, a protein is soluble if it contains no or few particles that can be removed by a filter with a pore size of 0.22 μm.

Further, it has been shown by the inventors that G-CSF can be produced in E. coli to a yield of approximately 3 mg/L culture and that G-CSF forms inclusion bodies when produced in E. coli. The protein designs according to the invention, on the other hand, can be produced as soluble proteins, i.e. without the formation of inclusion bodies, to significantly higher yields.

Thus, in certain embodiments, the invention relates to the G-CSF-like protein according to the invention, wherein the protein is expressed as soluble protein in E. coli. In particular, the invention relates to the protein according to the invention, wherein the protein is expressed as soluble protein in E. coli to a yield of at least 5 mg/L culture, at least 6 mg/L culture, at least 7 mg/L culture, at least 8 mg/L culture, at least 9 mg/L culture, at least 10 mg/L culture, at least 11 mg/L culture, at least 12 mg/L culture, at least 13 mg/L culture, at least 14 mg/L culture, at least 15 mg/L culture, at least 20 mg/L culture or at least 30 mg/L culture.

It is to be understood that the yields stated above refer to the yields that are obtained when expressing the G-CSF-like protein according to the invention in a shake flask. Expression of the G-CSF-like protein according to the invention in a continuous culture or in fermentation may result in higher yields.

The skilled person is aware of methods to express the G-CSF-like protein according to the invention in E. coli cells or in any other suitable microbial host cell. The expression of the protein according to the invention is further exemplified in FIG. 2.

That is, for expression of the G-CSF-like protein according to the invention, a preculture may be grown in LB medium, the cells may be collected, washed twice in PBS buffer, and resuspended in M9 minimal medium (240 mM Na₂HPO₄, 110 mM KH₂PO₄, 43 mM NaCl), supplemented with 10 μM FeSO₄, 0.4 μM H₃BO₃, 10 nM CuSO₄, 10 nM ZnSO₄, 80 nM MnCl₂, 30 nM CoCl₂ and 38 μM kanamycin sulfate, to an OD₆₀₀ of about 0.5 to 1. After 40 min of incubation at 25° C., 2.0 g ¹⁵N-labelled ammonium chloride (Sigma-Aldrich 299251) and 6.25 g ¹³C D-glucose (Cambridge Isotope Laboratories, Inc. CLM-1396) may be added in a 2.5 l culture. After another 40 min, IPTG may be added to a final concentration of 1 mM for overnight expression.

The skilled person is aware of methods to detect the formation of inclusion bodies. For example, the skilled person may analyze the soluble and insoluble fraction of cell lysates to detect the formation of inclusion bodies.

The protein according to the invention is characterized in that it has G-CSF-like activity. In general, G-CSF causes a wide range of cellular responses, which are initiated by the binding of G-CSF to the G-CSF receptor G-CSF-R. G-CSF-R ligand-binding is associated with dimerization of the receptor and signal transduction through proteins including Jak, Lyn, STAT, and Erk1/2. Within the present invention, “G-CSF-like activity” may refer to any activity of a protein that results in a similar response as the binding of G-CSF to the extracellular ligand-binding domain of G-CSF-R. Thus, a protein is said to have “G-CSF-like activity”, if it binds to the receptor G-CSF-R and activates one or more of the same cellular responses in a cell comprising the receptor G-CSF-R as binding of G-CSF to G-CSF-R does. The protein according to the invention has been designed in a way that the binding site that is involved in binding to G-CSF-R is preserved. Therefore, it is plausible, that the protein according to the invention binds to G-CSF-R and exhibits G-CSF-like activity in the sense of the present invention.

Preferably, a protein is said to exhibit G-CSF-like activity, if the protein exhibits at least one, more preferably at least two, even more preferably at least three, most preferably all of the following activities:
i) Induction of granulocytic differentiation of HSPCs;
ii) induction of the formation of myeloid colony-forming units from HSPCs;
iii) induction of the proliferation of NFS-60 cells; and/or
iv) activation of the downstream signaling pathways MAPK/ERK and/or JAK/STAT.

Within the present invention, a protein is said to have the potential to induce the granulocytic differentiation of hematopoietic stem and progenitor cells (HSPCs), if the protein can induce the differentiation of HSPCs into granulocytes, in particular into CD45⁺CD11b⁺CD15⁺, CD45⁺CD11b⁺CD16⁺ and/or CD45⁺CD15⁺CD16⁺ granulocytes. Example 6 shows that contacting HSPCs with the protein designs Boskar_3 (SEQ ID NO: 4), Boskar_4 (SEQ ID NO:5), Moevan (SEQ ID NO:6) and Disohair_2 (SEQ ID NO:19), respectively, resulted in the differentiation of HSPCs into CD45⁺CD11b⁺CD15⁺, CD45⁺CD11b⁺CD16⁺ and CD45⁺CD15⁺CD16⁺ granulocytes. Comparable cell counts and ratios between the respective cell types have been obtained for all protein designs when compared to recombinant G-CSF (FIGS. 8A-B and 9 A-B). These results demonstrate that the proteins according to the invention have the potential to induce the differentiation of HSPCs into granulocytes, in particular into CD45⁺CD11b⁺CD15+, CD45⁺CD11b⁺CD16⁺ and CD45⁺CD15⁺CD16⁺ granulocytes.

The skilled person is aware of methods to determine the potential of a protein to induce the differentiation of HSPCs into granulocytes. In particular, Example 6 provides a detailed protocol for testing the potential of a protein to induce the differentiation of HSPCs into granulocytes. A protein is said to induce the differentiation of HSPCs into granulocytes, if after contacting said protein with a population of HSPCs in a culture, at least 5%, at least 10%, at least 15%, at least 20%, at least 25% of the cells in the culture are CD45⁺CD11b⁺CD15+, and/or at least 5%, at least 10%, at least 15%, at least 20%, at least 25%, at least 30%, at least 35%, at least 40% of the cells in the culture are CD45⁺CD11b⁺CD16⁺ and/or at least 5%, at least 10%, at least 15%, at least 20% of the cells in the culture are CD45⁺CD15⁺CD16⁺.

The skilled person is aware of methods to determine if a cell comprises the surface proteins CD11b, CD15, CD16 and/or CD45. Preferably, the presence of these surface proteins is determined by staining the cells with fluorescently-labeled antibodies that specifically bind these surface proteins and subsequent analysis of the stained cells by flow cytometry methods such as FACS. The threshold for differentiating between cells that express the surface proteins and cells that do not express the surface proteins depend, amongst others, on the reagents and instruments that are used and thus may vary between experiments. However, the skilled person is capable of determining appropriate thresholds based on suitable negative and positive controls.

The protein may be added to the population of HSPCs in the culture at a concentration of less than 50 μg/mL, preferably less than 40 μg/mL, preferably less than 30 μg/mL, preferably less than 25 μg/mL, preferably less than 20 μg/mL, preferably less than 15 μg/mL, preferably less than 14 μg/mL, preferably less than 13 μg/mL, preferably less than 12 μg/mL, preferably less than 11 μg/mL to induce the differentiation of HSPCs into granulocytes.

The terms “human hematopoietic stem and progenitor cells” and “human HSPC” as used herein, include human self-renewing multipotent hematopoietic stem cells and hematopoietic progenitor cells.

The term “CD45”, as used herein refers to cluster of differentiation 45, which is also referred to as protein tyrosine phosphatase receptor type C (PTPRC) or leukocyte common antigen (LCA). CD45 is a type I transmembrane protein that is present in various isoforms on all differentiated hematopoietic cells.

The term “CD11b”, as used herein refers to cluster of differentiation 11b, which is also referred to as integrin alpha M. CD11b is expressed on the surface of many leukocytes involved in the innate immune system, including monocytes, granulocytes, macrophages, and natural killer cells.

The term “CD15”, as used herein refers to cluster of differentiation 15, which is also referred to as Sialyl-Lewisx or stage-specific embryonic antigen 1 (SSEA-1). CD15 is one of the most important blood group antigens and is displayed on the terminus of glycolipids that are present on the cell surface. CD15 is constitutively expressed on granulocytes and monocytes and mediates inflammatory extravasation of these cells.

The term “CD16”, as used herein refers to cluster of differentiation 16, which is also referred to as FcγRIIIb. CD16 is found on the surface of natural killer cells, neutrophils, monocytes, and macrophages.

In view of the above, a protein of the invention defined as “having G-CSF-like activity” may also be a protein that “induces the granulocytic differentiation of HSPCs” in an in vitro assay, preferably within 14 days. Accordingly, in one aspect, the proteins described herein and referred to as having “G-CSF-like activity” can alternatively be referred to as proteins that “induce the granulocytic differentiation of HSPCs” in an in vitro assay, preferably within 14 days, using any of the above-mentioned concentrations.

Within the present invention, a protein is said to have the potential to induce the formation of myeloid colony-forming units (CFUs) from HSPCs, if contacting of HSPCs with said protein results in the formation of at least one myeloid colony-forming unit. Example 7 shows that all tested protein designs, namely Boskar_3 (SEQ ID NO:4), Boskar_4 (SEQ ID NO:5), Moevan (SEQ ID NO:6) and Disohair_2 (SEQ ID NO:19) have the potential to induce the formation of myeloid CFUs when contacted with HSPCs (FIG. 10).

The skilled person is aware of methods to determine the potential of a protein to induce the formation of myeloid CFUs from HSPCs. In particular, Example 7 provides a detailed protocol for determining the potential of a protein to induce the formation of myeloid CFUs from HSPCs.

A protein is said to induce the formation of myeloid CFUs from HSPCs, if after contacting said protein with a population of HSPCs in a culture, at least one myeloid CFU is formed. In particular, a protein is said to induce the formation of myeloid CFUs from HSPCs, if after contacting said protein with a population of 10,000 HSPCs in a culture, at least 1, at least 2, at least 3, at least 4, at least 5, at least 6, at least 7, at least 8, at least 9, at least 10 myeloid CFUs are formed.

Preferably, the protein may be added to the population of HSPCs in the culture at a concentration of less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, to induce the formation of myeloid CFUs from HSPCs.

The term “myeloid CFU”, as used herein, refers to any colony forming unit that generates myeloid cells. Within the present invention, a myeloid CFU may preferably be a CFU-GEMM cell, a CFU-GM cell or a CFU-G cell.

In view of the above, a protein of the invention defined as “having G-CSF-like activity” may also be a protein that “induces the formation of myeloid CFUs from HSPCs” in an in vitro assay, preferably within 14 days. Accordingly, in one aspect the proteins described herein and referred to as having “G-CSF-like activity” can alternatively be referred to as proteins that “induce the formation of myeloid CFUs from HSPCs” in an in vitro assay, preferably within 14 days, using any of the above-mentioned concentrations.

Within the present invention, a protein is said to induce the proliferation of NFS-60 cells, if contacting NFS-60 cells in a culture with said protein results in an increased number of NFS-60 cells in the culture. As demonstrated in Example 5 (FIG. 5 and Table 5), the protein variants Boskar_1 (SEQ ID NO:2), Boskar_2 (SEQ ID NO:3), Boskar_3 (SEQ ID NO:4), Boskar_4 (SEQ ID NO:5), Moevan (SEQ ID NO:6), DiSohair_1 (SEQ ID NO:18), DiSohair_2 (SEQ ID NO:19) and Sohair (SEQ ID NO:14) have the potential to induce the proliferation of NFS-60 cells, which is a standard cell line for assaying human and murine G-CSF activity.

The skilled person is aware of methods to determine the potential of a protein to induce the proliferation of NFS-60 cells. The above-mentioned proliferation assay based on NFS-60 cells, as described in detail in Example 5 below, constitutes a common assay to determine G-CSF activity. The NFS-60 cell line is commercially available, for example from Cell Line Services GmbH. Within the present invention, a protein is determined to have G-CSF-like activity, if it induces proliferation of the population of NFS-60 cells in a culture at a half maximal effective concentration (EC50) of less than 100 μg/mL, preferably less than 50 μg/mL, preferably less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, preferably less than 1 μg/mL, preferably less than 0.75 μg/mL, preferably less than 0.5 μg/mL, preferably less than 0.25 μg/mL or preferably less than 0.1 μg/mL.

Thus, in one embodiment, G-CSF-like activity refers to the ability of a protein to induce the proliferation of NFS-60 cells, preferably in an assay as discussed above and in Example 5, below. It is widely accepted that only metabolically active cells are able to proliferate. Accordingly, proliferation of cells such as the NFS-60 cells may be measured by determining the metabolic activity of cells, e.g. by detecting the ability to reduce resazurin into resorufin in fluorescent assays. The skilled person is aware of methods to determine if cells in a culture are proliferating by measuring the metabolic activity of these cells [33]. “Inducing proliferation” in the context of proliferation assays using NFS-60 cells preferably means that the NFS-60 cells show after a certain time (for example 48 hours) a higher metabolic capacity (as, e.g., measured by detecting the reduction of resazurin into resorufin in a fluorescent assay) than a corresponding negative control in which the same amount of cells and the same medium is used with the only exception that no cytokine/protein to be tested is added. Alternatively, or additionally, a negative control may be a control protein (e.g. BSA etc.). The assay may preferably conducted as titration experiment in which increasing concentrations of the protein to be tested are added to the same amount of cells in the same volume medium in different wells (e.g. of a 96-well cell culture plate). In such a titration test, it is expected to identify a concentration range in which the proliferation and/or metabolic capacity increases in a concentration dependent manner. The assay may also involve a positive control, in which the same number of NFS-60 cells is incubated in the same type and volume of medium wild-type G-CSF (filgrastim), preferably also in different concentrations.

More specifically, an assay for measuring the potential of proteins to induce proliferation of NFS-60 cells may be conducted as follows. First, NFS-60 cells may be cultured in GM-CSF-containing RPMI 1640 medium ready-to-use, supplemented with L-glutamine, 10% KMG-5 and 10% FBS (cls, cell line services). These cells may be pelleted and washed three times with cold non-supplemented RPMI 1640 medium. After the last washing step, cells may be diluted at a density of 6×10⁵ cells/mL in RPMI 1640 medium containing 0.3 mg/mL glutamine and 10% FBS. In order to analyze cell proliferation, the resuspended NFS-60 cells may be distributed in cell culture plates (e.g. 96-well plates) and the protein(s) to be tested may be added at varying final concentrations (e.g. in the range from 0.000001 ng/ml to 1000 μg/ml). Optionally, each concentration may be tested in triplicates. The cell density may be adjusted to 3×10⁵ cells/mL in a well if 96-well plates are used. When using 96 well plates, these may contain triplicates for each protein concentration to be tested and the according blanks, including wells containing cells seeded in RPMI 1640 medium supplemented with L-glutamine, 10% KMG-5 and 10% FBS (cls, cell line services) and wells containing medium solely. In addition also positive controls using different concentration of wild type G-CSF (filgrastim) may be employed (e.g. varying from 0.00001-20 ng/mL). The cells may then be incubated for 48 h at 37° C. and 5% CO₂. After that incubation 30 μL of the redox dye resazurin (CellTiter-Blue® Cell Viability Assay, Promega) may be added to the wells and incubation may be continued for another hour. Cell viability can then be measured by monitoring the fluorescence of each well, e.g. by using a H4 Synergy Plate Reader (BioTek) using the following settings: excitation=560/9.0, Emission=590/9.0, read speed=normal, delay=100 msec, measurements/data Point=10. The data may then be analyzed and curves may be plotted applying a four-parameter sigmoid fit using SigmaPlot (Systat Software). What has been said above, regarding the cut-offs and measures to define a protein to have G-CSF-like activity according to this assay applies mutatis mutandis.

In view of the above, a protein of the invention defined as “having G-CSF-like activity” may also be a protein that “induces proliferation and/or metabolic capacity of NFS-60 cells” in an in vitro assay, preferably within 48 hours. Accordingly, in one aspect the proteins described herein and referred to as having “G-CSF-like activity” can alternatively be referred to as proteins that “induce proliferation and/or metabolic capacity of NFS-60 cells” in an in vitro assay, preferably within 48 hours, using any of the above-mentioned concentrations.

Thus, in a certain embodiment, the present invention relates to a protein comprising: a) one or two polypeptide chains; b) a bundle of four α-helices; and c) two or three amino acid linkers that connect contiguous bundle-forming α-helices that are located on the same polypeptide chain, wherein each amino acid linker has a length between 2 and 15 amino acids; wherein the protein induces the proliferation and/or metabolic capacity of NFS-60 cells.

In a certain embodiment, the present invention relates to a protein comprising: a) one or two polypeptide chains; b) a bundle of four α-helices; and c) two or three amino acid linkers that connect contiguous bundle-forming α-helices that are located on the same polypeptide chain, wherein each amino acid linker has a length between 2 and 15 amino acids; wherein the protein induces the proliferation and/or metabolic capacity of NFS-60 cells, in particular wherein the protein induces the proliferation and/or metabolic capacity of NFS-60 cells at a half maximal effective concentration (EC50) of less than 100 μg/mL, preferably less than 50 μg/mL, preferably less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, preferably less than 1 μg/mL, preferably less than 0.75 μg/mL, preferably less than 0.5 μg/mL, preferably less than 0.25 μg/mL or preferably less than 0.1 μg/mL.

Within the present invention, a protein is said to have the potential to activate the downstream signaling pathways MAPK/ERK and/or JAK/STAT, if contacting of cells, preferably HSPCs, with said protein results in the phosphorylation of the proteins ERK1, ERK2, STAT3, STAT5A and/or STAT5B. Example 8 shows that the protein design Moevan (SEQ ID NO:6) has the potential to increase the phosphorylation of the proteins STAT3, STAT5 and ERK1/2 (FIG. 11). Further, it is shown that the protein design DiSohair_2 (SEQ ID NO:19) has the potential to upregulate the phosphorylation of ERK1/2.

The skilled person is aware of methods to determine the potential of a protein to activate the downstream signaling pathways MAPK/ERK and/or JAK/STAT. In particular, Example 7 provides a detailed protocol for determining the potential of a protein to activate the downstream signaling pathways MAPK/ERK and/or JAK/STAT. A protein is said to activate the downstream signaling pathways MAPK/ERK and/or JAK/STAT, if after contacting said protein with a population of cells, preferably HSPCs, in a culture, the mean level of phosphorylated STAT3, STAT5 and/or ERK1/2 in the cells in the culture is increased. In particular, a protein is said to activate the downstream signaling pathways MAPK/ERK and/or JAK/STAT, if the mean level of phosphorylated STAT3, STAT5 and/or ERK1/2 in the cells of the culture is increased by at least 5%, preferably by at least 10%, preferably by at least 15%, preferably by at least 20%, preferably by at least 25% after contacting the cells in the culture with the protein for 10 minutes.

The skilled person is aware of methods to determine the phosphorylation level of a protein in a population of cells. Preferably, the phosphorylation level of a protein in a population is determined with antibodies against the phosphorylated protein. Prior to the addition of the antibodies, cells may be fixated and permeabilized by methods known in the art. The stained cells may then be analyzed by flow cytometry methods such as FACS to determine the level of phosphorylation of the protein. To determine the fold-change in phosphorylation upon contacting with the protein of the invention, phosphorylation levels may be compared between populations that have been contacted with the protein of the invention and populations that have not been contacted with the protein of the invention. Alternatively, the skilled person is aware of single-cell analysis methods to determine the degree of phosphorylation of a particular protein in a cell.

The protein may be added to the population of HSPCs in the culture at a concentration of less than 50 μg/mL, preferably less than 40 μg/mL, preferably less than 30 μg/mL, preferably less than 25 μg/mL, preferably less than 20 μg/mL, preferably less than 15 μg/mL, preferably less than 14 μg/mL, preferably less than 13 μg/mL, preferably less than 12 μg/mL, preferably less than 11 μg/mL, to activate the downstream signaling pathways MAPK/ERK and/or JAK/STAT.

In certain embodiments, the protein of the invention induces the phosphorylation of tyrosine 705 of STAT3. In other embodiments, the protein of the invention induces phosphorylation of tyrosine 694 of STAT5A. In other embodiments, the protein of the invention induces phosphorylation of tyrosine 699 of STAT5B. In other embodiments, the protein of the invention induces phosphorylation of threonine 202 of ERK1. In other embodiments, the protein of the invention induces phosphorylation of tyrosine 204 of ERK2.

The term “MAPK signalling pathway” is intended to mean a cascade of intracellular events that mediate activation of Mitogen-Activated-Protein-Kinase (MAPK) and homologues thereof in response to various extracellular stimuli. Three distinct groups of MAP kinases have been identified in mammalian cells: 1) extracellular-regulated kinase (ERK), 2) c-Jun N-terminal kinase (JNK) and 3) p38 kinase. The ERK MAP kinase pathway involves phosphorylation of ERK1 (p44) and/or ERK2 (p42). Activated ERK MAP kinases translocate to the nucleus where they phosphorylate and activate transcription factors including (Elk 1) and signal transducers and activators of transcription (Stat).

The term “JAK/STAT signaling pathway”, as used herein, refers a major signaling pathway comprising a receptor, Janus kinases (JAKs), and Signal Transducer and Activator of Transcription proteins (STAT). The JAK/STAT signaling pathway transmits information from chemical signals outside the cell into gene promoters on the DNA in the cell nucleus, causing DNA transcription and activity in the cell.

The receptor is activated by a signal from interferons, interleukins, growth factors, or other chemical messengers that induce phosphorylation of the receptor. STAT proteins may bind to the phosphorylated receptor, which can in turn induce their phosphorylation and oligomerization with other STAT proteins or further interaction proteins to then translocate into the cell nucleus. This oligomer forms a transcription factor that binds to DNA and promotes transcription of genes responsive to STAT.

STAT3 is a member of the STAT protein family. In response to cytokines and growth factors, STAT3 is phosphorylated by receptor-associated Janus kinases (JAK), form homo- or heterodimers, and translocate to the cell nucleus where they act as transcription activators. Specifically, STAT3 becomes activated after phosphorylation of tyrosine 705 in response to such ligands as interferons, G-CSF, epidermal growth factor (EGF), Interleukin (IL-)5 and IL-6. Additionally, activation of STAT3 may occur via phosphorylation of serine 727 by Mitogen-activated protein kinases (MAPK) and through c-src non-receptor tyrosine kinase. STAT3 mediates the expression of a variety of genes in response to cell stimuli, and thus plays a key role in many cellular processes such as cell growth and apoptosis.

Signal transducer and activator of transcription 5 (STAT5) refers to two highly related proteins, STAT5A and STAT5B, which are part of the seven-membered STAT family of proteins. Though STAT5A and STAT5B are encoded by separate genes, the proteins are 90% identical at the amino acid level. STAT5 proteins are involved in cytosolic signaling and in mediating the expression of specific genes.

In view of the above, a protein of the invention defined as “having G-CSF-like activity” may also be a protein that “activates the downstream signaling pathways MAPK/ERK and/or JAK/STAT” in an in vitro assay, preferably within 10 minutes. Accordingly, in one aspect the proteins described herein and referred to as having “G-CSF-like activity” can alternatively be referred to as proteins that “activate the downstream signaling pathways MAPK/ERK and/or JAK/STAT” in an in vitro assay, preferably within 10 minutes, using any of the above-mentioned concentrations.

G-CSF-like activity of a protein may also or in addition be measured indirectly by analyzing the binding of said protein to the receptor G-CSF-R. The skilled person is aware of methods to measure the binding affinity of a protein to G-CSF-R or to determine if a protein is in competition for G-CSF-R with a known ligand, such as G-CSF. A widely used and reliable means for measuring the binding affinity between two molecules, for example a protein and a ligand, is isothermal titration calorimetry [36]. Further, the skilled person is aware of methods to quantitatively measure signal transduction events induced by G-CSF treatment of cells expressing G-CSF-R to measure receptor binding by downstream signal transduction. In addition, the skilled person is aware of computational methods that allow simulating the binding of a protein to a receptor.

It has to be noted that certain G-CSF-like activities were only achieved with the protein according to the invention when significantly higher concentrations compared to recombinant human G-CSF were applied. However, this lower activity of the protein according to the invention compared to recombinant human G-CSF may be compensated by the more efficient production process of the protein according to the invention, i.e. higher production yields and no need for refolding of insoluble protein. On the other hand, the lower activity of the protein according to the invention may even have beneficial effects in therapy, and may, for example, result in delayed action of the protein after administration to a patient and/or in reduced side effects caused by excessive granulopoiesis. Medical indication where a lower and/or long-lasting G-CSF-like activity may be desirable are inherited neutropenias and/or chemotherapy-induced neutropenia.

The term “protein” as used herein, describes a macromolecule comprising one or more polypeptide chains. A “polypeptide chain” is a linear chain of amino acids, wherein the contiguous amino acids are connected by peptide bonds. Polypeptide chains preferably consist of the 20 canonical amino acids, but may also comprise non-canonical amino acids. “Non-canonical amino acids” are all amino acids that do not belong to the 20 standard amino acids of the genetic code.

The secondary structure is the three dimensional form of local segments of proteins or polypeptide chains. The two most common secondary structural elements are α-helices and β-sheets, though β-turns and omega loops occur as well. Secondary structural elements typically spontaneously form as an intermediate before the protein or polypeptide chain folds into its three dimensional tertiary structure.

The tertiary structure is the three dimensional shape of a protein or polypeptide chain. The tertiary structure of a protein is the three dimensional arrangement of multiple secondary structures belonging to a single polypeptide chain. Amino acid side chains may interact in different ways including hydrophobic interactions, salt bridges, hydrogen bonds, van der Waals forces and covalent bonds. The interactions and bonds of side chains within a particular protein or polypeptide chain determine its tertiary structure. The tertiary structure is defined by its atomic coordinates. A number of tertiary structures may fold into a quaternary structure.

The term “α-helix” as used herein, indicates a right-handed spiral conformation of a polypeptide chain or of a part of a polypeptide chain. In an α-helix, every backbone N—H group donates a hydrogen bond to the backbone C═O group of the amino acid three or four residues earlier along the polypeptide chain.

A “bundle of four α-helices” as used herein, is defined as a protein fold composed of four α-helices that are nearly parallel or antiparallel to each other. An α-helix that contributes to the bundle of four α-helices is called a “bundle-forming α-helix”. The four α-helices that form the bundle of four α-helices may be located on a single polypeptide chain or may be located on two or more separate polypeptide chains. An amino acid linker connects two α-helices that are located on the same polypeptide chain. The term “amino acid linker” as used herein, refers to a sequence of amino acids that is located between the C-terminal end of a first α-helix and the N-terminal end of a second α-helix, wherein the amino acids of the amino acid linkers are not part of any of the α-helices. Two α-helices are said to be contiguous, if they are located on the same polypeptide chain and are directly connected by an amino acid linker. The length of an amino acid linker is defined as the number of amino acid residues that constitute the linker.

The term “amino acid sequence” as used herein, refers to the sequence of amino acid residues of a protein. The amino acid sequence is usually reported in an N-to-C-terminal direction. The term “sequence identity,” as used herein, is generally expressed as a percentage and refers to the percent of amino acid residues that are identical between two sequences when optimally aligned. For the purposes of this invention, sequence identity means the sequence identity determined using the well-known Basic Local Alignment Search Tool (BLAST), which is publicly available through the National Cancer Institute/National Institutes of Health (Bethesda, Md.) and has been described in printed publications [17]. Preferred parameters for amino acid sequences comparison using BLASTP are gap open 11.0, gap extend 1, Blosum 62 matrix.

In certain embodiments of the present invention, the G-CSF-like protein according to the invention is more stable than G-CSF. This higher stability has the advantage that the protein according to the invention has a higher shelf life and does not necessarily require a cold supply chain. The term “stability” as used herein, refers to the ability of a molecule, for example a protein, to maintain a folded state under physiological conditions such that it retains at least one of its normal functional activities, for example, binding to a target molecule such as a receptor. The skilled person is aware of methods to determine the stability of a protein. Methods for determining protein stability comprise, but are not limited to differential scanning calorimetry, differential scanning fluorometry, pulse-chase methods, bleach-chase methods, cycloheximide-chase methods, circular dichroism spectroscopy, fluorescence-based activity assays, Fourier Transform Infrared Spectroscopy, and various computer-based prediction methods. Stability of a protein can be influenced by many factors, such as temperature, salt concentration, pH and the presence of proteases. A protein is said to be “thermally instable” if the protein is susceptible to denaturation at elevated temperatures. On the other hand, a protein is said to be “thermally stable” or “thermostable” if the protein can resist relatively high temperatures without denaturing.

For example, the thermal stability of a protein may be quantified by determining the temperature at which the protein is fully denatured. A protein is “fully denatured”, if it has completely lost any quaternary, tertiary, and/or secondary structure that is originally present in the native or non-denatured protein. A protein that is not fully denatured is said to be partially or completely folded. The temperature at which a protein is fully denatured depends on various factors, for example, the solvent and buffer conditions, a bound ligand, pressure and the temperature ramp rate that is applied to the protein. Within the present invention, the thermal stability of the protein variants of the invention and G-CSF was tested in a buffer comprising phosphate buffered saline, pH 7.4 and the temperature was increased at a rate of 1 K (Kelvin) per minute. Under these conditions, G-CSF was shown to have the denaturation midpoint at a temperature of approximately 330 K (Kelvin). Thus, a G-CSF-like protein is determined to be more stable than G-CSF, if it remains partially or completely folded at temperatures above 330 K, preferably 335 K, preferably 340 K, preferably 345 K, preferably 350 K, preferably 355 K, preferably 360 K, preferably 365 K or preferably 370 K under the conditions used within the present invention. Alternatively, also other conditions may be employed and the melting temperature of G-CSF and the protein according to the invention may be measured under the same conditions. The melting temperature (Tₘ) may be extracted from a melting curve and corresponds to the temperature at which 50% of the protein is unfolded (see Example 3 for an exemplary embodiment to define the Tₘ). Accordingly, the melting temperature is defined as the melting curve inflection mid-point. A G-CSF-like protein is then classified thermally more stable than G-CSF if the melting temperature measured in ° C. is at least 5%, preferably 10%, even more preferably 15%, even more preferably 20%, and most preferably 25% higher than the melting temperature of a G-CSF reference under the same experimental conditions. Alternatively, in certain embodiments, the G-CSF-like protein according to the invention is classified thermally more stable than G-CSF if it has a melting temperature of more than 57° C., preferably more than 60° C., even more preferably more than 65° C., most preferably more than 70° C. It is to be understood that melting temperatures disclosed herein are melting temperatures at neutral pH. More particularly, the melting temperatures disclosed herein are melting temperatures in 1×PBS (137 mM NaCl, 10 mM Phosphate, 2.7 mM KCl, and a pH of 7.4).

Engineered G-CSF analogs with a higher thermal stability have been reported in the art. For example, Luo et al. reported an engineering approach which increased the melting temperature of human G-CSF from 60° C. to 73° C. at neutral pH [40]. In another approach, Miyafusa et al. reported an engineered G-CSF analog with a melting temperature at neutral pH of 69.4° C. compared to less than 60° C. for human G-CSF [10]. Of the protein designs disclosed herein, Moevan has a melting temperature of 74° C. The designs Boskar_4 and DiSohair2 have melting temperatures above 100° C. (Table 6). Accordingly, the protein designs of the present invention have higher thermal stabilities than the G-CSF analogs disclosed in the prior art.

Accordingly, in certain embodiments, the present invention relates to a protein comprising: a) one or two polypeptide chains; b) a bundle of four α-helices; and c) two or three amino acid linkers that connect contiguous bundle-forming α-helices that are located on the same polypeptide chain, wherein each amino acid linker has a length between 2 and 15 amino acids; wherein the protein has G-CSF-like activity and wherein the protein has a melting temperature (Tₘ) of at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C. or at least 95° C.

Alternatively, in certain embodiments, the present invention relates to a protein comprising: a) one or two polypeptide chains; b) a bundle of four α-helices; and c) two or three amino acid linkers that connect contiguous bundle-forming α-helices that are located on the same polypeptide chain, wherein each amino acid linker has a length between 2 and 15 amino acids; wherein the protein comprises one or more G-CSF receptor binding sites and wherein the protein has a melting temperature (Tm) of at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C. or at least 95° C.

More specifically, an assay for determining the thermal stability of a protein may be conducted as follows. Thermal unfolding may be measured by CD spectroscopy monitoring the loss of secondary structure, wherein the temperature may be monitored and regulated by a Peltier element which may be connected to the CD spectroscopy unit. The temperature may be measured in the cuvette jacket made of copper. Samples (0.5 mL) with concentrations between 0.3 and 6 mg/mL of the respective proteins in 1×PBS buffer (pH 7.4) may be loaded into 2 mm path length cuvettes. Spectral scans of mean residual ellipticity may be measured at a resolution of 0.1 nm, across the range of 240-195 nm. The mean residual ellipticity at a wavelength of 222 nm across a temperature range of 20 to 100° C. (with an increase of 1° C. per minute) may be tracked in a melting curve. The melting temperature may be extracted as the value of Tₘ (where

$$\frac{1}{2} = \frac{{T}_{max} - {T}_{m}}{{T}_{max} - {T}_{min}}$$

), where an inflection is observed. The temperature at which a protein is fully denatured may be extracted as the temperature after the melting inflection with the maximum mean residual ellipiticity, Tₘₐₓ.

The term “protease” as used herein is an enzyme that hydrolyzes peptide bonds (has protease activity). Proteases are also called e.g. peptidases, proteinases, peptide hydrolases, or proteolytic enzymes. A protein or peptide is said to have a “higher stability in the presence of a protease” compared to a second protein or peptide, if the first protein or peptide has a higher potential to maintain a correctly folded state in the presence of the protease. Example 4 shows that some of the protein variants of the present invention are more stable in the presence of the protease neutrophil elastase. Neutrophil elastase is a serine protease that has broad substrate specificity. Secreted by neutrophils and macrophages during inflammation, neutrophil elastase enzymatically antagonizes G-CSF activity as well as it destroys virulence factors and other outer membrane proteins of bacteria and extracellular matrix molecules, including collagen-IV and elastin, of the host tissue. It also localizes to Neutrophil extracellular traps (NETs), via its high affinity for DNA, an unusual property for serine proteases. Without being bound to theory, it is to be expected that proteins with a higher stability in the presence of neutrophil elastase have a longer circulation half-life in blood and, therefore, improved therapeutic efficacy. Thus, in certain embodiments, the invention relates to a protein according to the invention, wherein the protein has a higher stability in the presence of proteases, preferably neutrophil elastase, compared to human G-CSF.

The term “circulation half-life” as used herein, refers to the time required for half of a quantity of the protein according to the invention to be eliminated in blood circulation.

Certain embodiments of the present invention relate to a G-CSF-like protein according to the invention that is produced more efficiently than G-CSF. The term “production level” as used herein in reference to proteins, refers to the amount of recombinant protein that is produced by a defined number of cells. The production level is most frequently expressed as the amount of purified protein, usually given in grams, that is obtained per volume of cell culture, usually given in liters, containing a defined number of cells.

The G-CSF-like protein according any embodiment may be produced in a cell. The term “cell” as used herein is seen to include all types of eukaryotic and prokaryotic cells and further includes naturally occurring, unmodified cells as well as genetically modified cells and cell lines. The term “cell line” as used herein shall mean an established clone of a particular cell type that has acquired the ability to proliferate over a prolonged period of time, specifically including immortal cell lines, cell strains and primary cultures of cells. Cells that are particularly suitable for the expression of proteins are bacteria, such as Escherichia coli or species from the genera Salmonella, Bacillus, Corynebacterium or Pseudomonas, yeasts, such as Saccharomyces cerevisiae or Pichia pastoris, filamentous fungi from the genera Aspergillus, Trichoderma or Myceliophtora, insect cell lines, such as Sf9, Sf21 or High Five, or mammalian cell lines, such as HeLa, CHO or HEK 293 cells. Bacterial cells, yeasts and fungi may be summarized as microbial cells. The cells that are used for the production of the protein according to the invention may be cultured in any suitable culture vessel or bioreactor.

The G-CSF-like protein variants of the present invention have been synthesized in the bacterium Escherichia coli that is also used as production host of the recombinant human G-CSF variant filgrastim. One advantage of the protein variants of the present invention in comparison to filgrastim is that the protein variants of the invention are expressed as soluble proteins that can be directly purified from cell lysates. Filgrastim, on the other hand, forms aggregates in the form of inclusion bodies when expressed in E. coli, and needs to be re-solubilized before it can be purified FIG. 7 exemplary shows the expression profiles of G-CSF and the protein designs Moevan (SEQ ID NO:6) and Disohair_1 and 2 (SEQ ID NOs:18 and 19). While the protein designs Moevan and Disohair are clearly detectable in the soluble fraction of a cell lysate, only traces of G-CSF are detectable in the soluble fraction. In addition, the protein variants of the invention are produced at higher levels compared to filgrastim, which was previously reported to be produced with a yield of 3.2 mg of bioactive protein per liter of cell culture [11]. After sequential purification through IMAC and size exclusion chromatography, the yield was at least 4 times higher for the designed variants compared to the recombinantly expressed (Table 6). Thus, in certain embodiments, the invention relates to a protein according to the invention, wherein the protein is produced more efficiently than human G-CSF in a host cell, preferably a microbial host cell, more preferably a bacterial host cell, most preferably E. coli.

In another embodiment, the invention relates to a protein according to the invention, wherein the protein comprises one or more G-CSF receptor binding sites.

Accordingly, in certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites.

In certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein the protein has a melting temperature of at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C. or at least 95° C.

The residues of G-CSF that are involved in binding to G-CSF-R have previously been identified by site-directed mutagenesis and X-ray crystallography [26, 27]. The protein according to the invention may be designed such that the spatial orientation, electrostatic and hydrophobic features of the binding site of G-CSF that is involved in the binding to G-CSF-R is preserved. Accordingly, the most relevant amino acid residues of G-CSF involved in the binding to G-CSF-R, or amino acid residues with similar features, may be mapped on the protein of the invention such that these amino acid residues have a similar spatial orientation to each other as in G-CSF (see below for further details). Due to this design constraint, it is plausible that the protein according to the invention binds and activates the receptor G-CSF-R, despite the fact that the protein has only little to no sequence homology with G-CSF over the whole length of the protein. The protein according to the invention may have one G-CSF-R binding site, or may have more than one G-CSF-R binding site.

The G-CSF-like protein according to the invention has been designed in a way, such that it can bind and activate the receptor G-CSF-R. The term “binding site”, as used herein, refers to one or more regions of a molecule or macromolecular complex, for example a protein that, as a result of its shape, favorably associate with another chemical entity or compound. A “G-CSF receptor binding site” as used herein, refers to one or more regions of a protein that favorably associate with the extracellular ligand-binding portion of the receptor G-CSF-R, such that G-CSF-R is activated. The shape of a protein-based binding site is determined by a set of amino acids with specific molecular interaction features and a defined spatial arrangement towards each other. In case of human G-CSF, the site II amino acid residues that more than doubled the EC₅₀ when replaced with an Alanine were Lysine 16, Glutamate 19, Glutamine 20, Arginine 22, Lysine 23, Aspartate 27, Aspartate 109 and Aspartate 112 have been reported to be the residues that form the G-CSF receptor binding site [26]. The protein designs Moevan (SEQ ID NO:6-13 and 20-22), Sohair (SEQ ID NO:14-17 and 23-25), Disohair (SEQ ID NO:18-19) have been designed in a way that the spatial and electrostatic features of at least 6 of the amino acid residues Lysine 16, Glutamate 19, Glutamine 20, Arginine 22, Lysine 23, Aspartate 27, Aspartate 109, and Aspartate 112 of G-CSF are preserved in the protein according to the invention (see highlighted residues in Table 5). In the Boskar design (SEQ ID NO:2-5) all these residues were maintained (see highlighted residues in Table 5). Thus, in a more preferred embodiment, the invention relates to a G-CSF-like protein according to the invention, wherein the spatial orientation and molecular interaction features of at least two, at least three, at least four, at least five, at least six, at least seven or most preferably all of the amino acid residues Lysine 16, Glutamate 19, Glutamine 20, Arginine 22, Lysine 23, Aspartate 27, Asparagine 109, and Aspartate 112 of G-CSF are preserved.

Two or more amino acid residues in a protein are said to be “preserved” between two proteins, if they have similar spatial orientation and molecular interaction features in both proteins. “Spatial orientation”, as used herein, refers to the relative C-alpha positions of the residues and their associated C-alpha-C-beta vectors, which define their side chain orientation. Two or more amino acid residues from individual proteins are determined to have similar spatial orientation, if the residues have a C-alpha-based root-mean square deviation of less than 4 Angstroms, preferably less than 3 Angstroms, more preferably less than 2 Angstroms, most preferably less than 1 Angstrom.

The skilled person is aware of methods to determine C-alpha-based root-mean square deviation of two or more residues from individual proteins [34]. Within the present invention, certain amino acid residues of the protein according to the invention may have a similar spatial orientation as their corresponding amino acid residues in human G-CSF. Accordingly, the G-CSF-like protein according to the invention comprises at least four, preferably at least five, more preferably at least six, even more preferably at least seven, most preferably eight amino acids residues that have a similar special orientation as the amino acid residues Lysine 16, Glutamate 19, Glutamine 20, Arginine 22, Lysine 23, Aspartate 27, Aspartate 109, and Aspartate 112 of human G-CSF.

Example 10 describes a method to determine the spatial orientation of the amino acid residues in the G-CSF binding epitope. For that, the three-dimensional structure of a protein in question has to be determined. Methods for determining the three-dimension structure of a protein are known in the art and preferably involve NMR spectroscopy or X-ray crystallography. However, three-dimensional structures of proteins may also be determined by computational methods. Various three-dimensional structures of human G-CSF have been disclosed and are freely available to the person skilled in the art.

Various computational tools are known in the art to compare the structure of a protein of interest with the structure of human G-CSF. One method commonly known in the art for comparing the spatial orientation of one or more amino acid residues in a protein is the CoMAND method (Conformational Mapping by Analytical NOESY Decomposition) (see Example 10 and FIG. 17B).

The electrostatic features of an amino acid residue may be determined by their side chain or by the atoms of the peptide backbone, which may both be involved in intramolecular or intermolecular interactions, such as salt bridges, hydrogen bonds, and charge-dipole interactions, Pi-effects, hydrophobic effect, and Van der Waals forces. Amino acid residues with similar electrostatic features are preferably identical, but may also be other closely related amino acids.

Within the present invention, one or more of the amino acid residues Lysine 16, Glutamate 19, Glutamine 20, Arginine 22, Lysine 23, Aspartate 27, Asparagine 109, and Aspartate 112 of G-CSF may be substituted with another amino acid residue. Preferably, said amino acid residues may be replaced with closely related amino acid residues. Substituting an amino acid residue with a closely related amino acid residue is called a conservative substitution. Conservative substitutions are shown in Table 1 below under the heading of “preferred substitutions”. More substantial changes are provided in Table 1 below under the heading of “exemplary substitutions”, and as further described below in reference to amino acid side chain classes.

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

In certain embodiments, a glutamate residue may be replaced with an aspartate residue or vice versa. In certain embodiments, a glutamine residue may be replaced with an asparagine residue or vice versa. Amino acids may further be replaced with non-canonical amino acids, in particular non-canonical amino acids with similar electrostatic features. For example, lysine residues may be replaced, without limitation by ornithine. Similarly, arginine residues may be replaced, without limitation, by homo-arginine.

Non-conservative substitutions may also entail exchanging a member of one of these groups for another group.

Accordingly, in certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein each G-CSF receptor binding site individually comprises at least four, preferably at least five, more preferably at least six, even more preferably at least seven, most preferably eight amino acid residues having a similar structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

Preferably, in certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein each G-CSF receptor binding site individually comprises six to eight amino acid residues having a similar structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

More preferably, in certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein each G-CSF receptor binding site individually comprises eight amino acid residues having a similar structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

In certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein each G-CSF receptor binding site individually comprises at least four, preferably at least five, more preferably at least six, even more preferably at least seven, most preferably eight amino acid residues having an identical structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

Preferably, in certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein each G-CSF receptor binding site individually comprises six to eight amino acid residues having an identical structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

More preferably, in certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein each G-CSF receptor binding site individually comprises eight amino acid residues having an identical structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

It is to be understood that, within the present invention, the residues Lysine 16, Glutamate 19, Glutamine 20, Arginine 22, Lysine 23, Aspartate 27, Aspartate 109, and Aspartate 112 of human G-CSF form the G-CSF-R binding epitope. This view is supported by the findings of Young et al. [26]. In certain embodiments, any of the proteins disclosed herein may comprise further epitope-proximal residues of human G-CSF. Epitope-proximal residues of human G-CSF particularly comprise residues Leucine15, Leucine 108, Threonine 115 and Threonine 116.

Thus, in certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein each G-CSF receptor binding site individually comprises at least four, preferably at least five, more preferably at least six, even more preferably at least seven, even more preferably at least eight, even more preferably at least nine, even more preferably at least ten, even more preferably at least 11, most preferably twelve amino acid residues having a similar or identical structure and a similar special orientation towards each other as the amino acid residues Leucine 15, Lysine 16, Glutamate 19, Glutamine 20, Arginine 22, Lysine 23, Aspartate 27, Leucine 108, Aspartate 109, Aspartate 112, Threonine 115 and Threonine 116 of human G-CSF.

in certain embodiments, the present invention relates to a protein comprising: a) one or two polypeptide chains; b) a bundle of four α-helices; c) two or three amino acid linkers that connect contiguous bundle-forming α-helices that are located on the same polypeptide chain, wherein each amino acid linker has a length between 2 and 15 amino acids; and d) one or more G-CSF receptor binding sites; wherein each G-CSF receptor binding site individually comprises eight amino acid residues having a similar or identical structure and a similar special orientation towards each other as the amino acid residues Lysine 16, Glutamate 19, Glutamine 20, Arginine 22, Lysine 23, Aspartate 27, Aspartate 109 and Aspartate 112 of human G-CSF and at least one, preferably at least two, more preferably at least three or most preferably four amino acid residues having a similar or identical structure and a similar special orientation towards each other as the amino acid residues Leucine 15, Leucine 108, Threonine 115 and Threonine 116 of human G-CSF.

It has been demonstrated herein that the proteins of the invention directly bind to G-CSF-R. In particular, Example 11 discloses dissociation constants between the protein designs and G-CSF-R in the low-micromolar or even nanomolar range. Thus, it has been convincingly shown for at least four designs without significant overall sequence homologies that the correct orientation of only six to eight amino acid residues that mimic the binding epitope of G-CSF is sufficient to achieve specific binding of a protein to G-CSF-R.

Accordingly, in certain embodiments, the present invention relates to a protein according to the invention, wherein the protein binds to G-CSF-R with a binding affinity of less than 1 mM, less than 900 μM, less than 800 μM, less than 700 μM, less than 600 μM, less than 500 μM, less than 400 μM, less than 300 μM, less than 200 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM or less than 1 μM.

Alternatively, in certain embodiments, the present invention relates to a protein according to the invention, wherein the protein binds to G-CSF-R with a binding affinity ranging from 0.1 nM to 1 mM, from 0.1 nM to 500 μM, ranging from 0.1 nM to 100 μM, ranging from 0.1 nM to 50 μM, ranging from 0.1 nM to 25 μM, ranging from 0.1 nM to 10 μM, ranging from 0.5 nM to 10 μM or ranging from 1 nM to 10 μM.

The term “binding affinity” as used herein refers to the strength of the non-covalent interaction between two molecules, e.g., a single binding site on the protein of the invention and a target, e.g., G-CSF-R, to which it binds. Thus, for example, the term may refer to 1:1 interactions between a protein and its target, unless otherwise indicated or clear from context. Binding affinity may be quantified by measuring an equilibrium dissociation constant (K_{d}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system (for example, using the method described in Example 11 below); kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{d}'s are calculated that may reflect avid binding.

The binding affinity may be determined by any method known in the art, in particular as described in Example 11.

In yet another embodiment, the invention relates to a G-CSF-like protein according to the invention, wherein the protein induces the proliferation and/or differentiation of cells comprising one or more G-CSF receptor on the cell surface.

G-CSF is a growth factor that induces, amongst others, but not exclusively, the proliferation and differentiation of myeloid cells, in particular neutrophil and basophil progenitors, both in vitro and in vivo. These processes are triggered by the activation of the receptor G-CSF-R, which is initiated by the binding of G-CSF to the receptor. Since the amino acids of G-CSF that are involved in the binding to G-CSF-R are preserved in the protein according to the invention, it is plausible to assume that the protein according to the invention induces the same biological functions as G-CSF. Thus, the protein according to the invention may induce the proliferation and/or differentiation of any cell that comprises one or more G-CSF receptor on its cell surface.

This cell may be, but is not limited to, a hematopoietic stem cell or any cell deriving thereof, a common myeloid progenitor or any cell deriving thereof, or a myeloblast or any cell deriving thereof. Thus, in a preferred embodiment, the invention relates to a protein according to the invention, wherein the protein induces the proliferation and/or differentiation of a cell that comprises one or more G-CSF receptors on its surface, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof. In Example 5 (FIG. 5 and Table 5) it is demonstrated that the protein according to the invention can induce the proliferation of the myeloblastic cell line NFS-60.

The term “proliferation” as used herein, refers to a rapid and repeated succession of divisions of cells over a period of time. Thus, a molecule is determined to “induce the proliferation of a cell”, if said molecule has the potential to induce the rapid and repeated succession of divisions of said cell over a period of time. The skilled person is aware of methods to determine if a molecule has the potential to induce the proliferation of a cell. Corresponding methods are described herein elsewhere. Within the present invention, the cell line NFS-60 may be used to determine the potential of the protein variants of the present invention to induce cell proliferation as described herein elsewhere. In particular, proliferation of cells, such as the NFS-60 may be measured by measuring metabolic activity of cells as explained herein elsewhere.

The term “differentiation” as used herein, refers to the process by which a less specialized cell becomes a more specialized cell. Thus, a molecule is determined to “induce the differentiation of a cell”, if said molecule has the potential to induce the specialization of a less specialized cell into a more specialized cell. The potential of a molecule to induce cell differentiation may be determined by incubating a less specialized cell in a solution comprising the molecule of interest. Within the present invention, the less specialized cells are preferably stem cells and/or progenitor cells that have been isolated from bone marrow, peripheral blood or umbilical cord blood. The skilled person is aware of methods to determine if a molecule can induce proliferation of a cell. For example, the differentiation level of a cell may be determined by measuring the expression levels of suitable reporter genes. A reporter gene may be any gene that is differentially expressed between cells with different differentiation levels. Within the present invention, the stage of granulopoiesis of cells, in particular human bone marrow stem cells, in a culture may, for example, be determined by quantifying the levels of the ELA2 mRNA or the ELA2 protein expressed by the cells via qRT-PCR or Western Blot [35]. In addition, the stage of granulopoiesis of cells, in particular human bone marrow stem cells, may be determined by quantifying the CXCR4 expression on the cell surface, for example by fluorescence-assisted cell sorting [35].

The term “cell surface” as used herein, refers to the extracellular part of the outer barrier of a cell, preferably the cell membrane. A receptor is said to be located on the cell surface, if the receptor is anchored to the cell membrane, preferably in a way that it is displayed on the extracellular side of the cell membrane.

NFS-60 is a murine myeloblastic cell line established from leukemia cells obtained after infection of (NFS×DBA/2) F1 adult mice with Cas Br-M murine leukemia virus. NFS-60 cells are dependent on IL-3 for growth and maintenance of viability in vitro. These cells are used to assay murine and human G-CSF. This bipotential murine hematopoietic cell line is responsive to IL-3, GM-CSF, G-CSF, and erythropoietin. The NFS-60 cell line is commercially available, for example from Cell Line Services GmbH (https://clsgmbh.de/).

In another embodiment, the invention relates to a G-CSF-like protein according to the invention, wherein the calculated contact order number of said protein is lower than the calculated contact order number of human G-CSF.

It is generally assumed that the folding rate of a protein is related to the thermal stability of the protein. Without being bound to theory, faster protein folding reduces the risk of misfolding and aggregation, and thereby leads to the formation of proteins with higher stability. A common method to estimate the folding rate of a protein is to calculate the contact order number of the protein. The “contact order number” of a protein, as used herein, is a measure of the locality of the inter-amino acid contacts in the protein's native state tertiary structure. It is calculated as the average sequence distance between residues that form native contacts in the folded protein divided by the total length of the protein. Higher contact order numbers indicate longer folding time, and low contact order numbers have been suggested as a predictor of potential downhill folding, or protein folding that occurs without a free energy barrier. The contact order number may be calculated as described by Plaxco et al. [20].

For G-CSF (SEQ ID NO:1; PDB file 5GW9), an absolute contact order number of 18.6 was calculated (Table 4). The exemplary protein variants of the invention presented in the appended examples have lower absolute contact order numbers than G-CSF, with values ranging between 4.5 and 17.8. For the reasons stated above, and again without being bound to theory, faster folding proteins are likely to be more (kinetically) stable than slower folding proteins. Thus, in a preferred embodiment, the invention relates to a G-CSF-like protein according to the invention, wherein the calculated absolute contact order number is lower than 18.6, preferably between 4 and 18, most preferably between 4.5 and 17.85. Preferred contact order numbers are the values indicated in Table 4 for the exemplary proteins of the invention.

In yet another embodiment, the invention relates to a G-CSF-like protein according to the invention, wherein the protein has a molecular mass between 13 and 18 kDa.

The term “molecular mass” as used herein, refers to the mass of a molecule. It is calculated as the sum of the relative atomic masses of each constituent element multiplied by the number of atoms of that element in the molecular formula. The molecular mass of a protein is usually expressed in the unit Dalton.

Human G-CSF, including the O-linked glycosyl group at position threonine 133, has a molecular mass of 19.6 kDa [13]. Filgrastim, a non-glycosylated, recombinant human G-CSF variant produced in E. coli, has a molecular mass of 18.8 kDa. Several approaches have been carried out to generate more stable G-CSF variants, but none of these variants resulted in proteins with significantly reduced molecular mass. PEGylation of filgrastim, for example, significantly increases the molecular mass of the protein. Accordingly, the PEGylated filgrastim variant pegfilgrastim, for example, comprises a 20 kDa PEG molecule attached to filgrastim [8]. Glycine-to-alanine scanning is also expected to result in G-CSF variants with slightly higher molecular mass, due to the higher molecular mass of alanine compared to glycine. Only the circularization of G-CSF, which resulted in the deletion of up to 11 amino acid residues from the terminal ends of G-CSF resulted in G-CSF variants with a slightly lower molecular mass of 17.6 kDa [10].

The G-CSF-like protein according to the invention may have a lower molecular mass compared to human G-CSF. Accordingly, the Boskar and Moevan protein variants (SEQ ID NO:2-13 and 20-22) have molecular masses between 13 and 14 kDa, respectively. The Sohair protein variants (SEQ ID NO:14-17 and 23-25) have a molecular mass of approximately 17.9 kDa and the Disohair protein variants (SEQ ID NO:18-19), consisting of two polypeptide chains, have a combined molecular mass of 17.7 kDa. Thus, all protein variants of the invention have a lower molecular mass than human G-CSF or the recombinant human G-CSF variant filgrastim. Accordingly, in an alternative embodiment, the invention relates to a G-CSF-like protein according to the invention, wherein the protein has a lower molecular mass than human G-CSF.

In a further embodiment, the invention relates to a G-CSF-like protein according to the invention, wherein the protein comprises no disulfide bonds.

The term “disulfide bond” as used herein, refers to a covalent bond formed between two sulfur atoms. Within a protein or peptide, the amino acid cysteine comprises a thiol group that can form a disulfide bond with a second thiol group, for example from a second cysteine residue. Previous approaches to obtain G-CSF variants with increased thermal stability that have been discussed above use human G-CSF as a template and still have very high sequence homology with human G-CSF. Consequently, these variants possess all five cysteine residues of G-CSF, of which four are involved in the formation of disulfide bonds. The inherent problem in the process of disulfide bond formation is that the mis-pairing of cysteines can cause misfolding, aggregation and ultimately result in low yields during protein production. To circumvent this problem and to obtain higher production levels, the protein according to the invention may be essentially free of disulfide bonds. The absence of disulfide bonds in the proteins of the present invention is guaranteed by the fact that none of the protein variants of the present invention comprises cysteine residues. Thus, in an alternative embodiment, the invention relates to a protein according to the invention, wherein the protein is free of cysteine residues.

In another embodiment, the invention relates to a G-CSF-like protein according to the invention, wherein the protein is not glycosylated.

The term “glycosylation” as used herein refers to the addition of a glycosyl group, usually to, but not limited to, an arginine, an asparagine, a cysteine, a hydroxylysine, a serine, a threonine, a tyrosine, or a tryptophan residue of a protein, resulting in a glycoprotein. A glycosyl group refers to a substituent structure obtained by removing the hemiacetal hydroxyl group from the cyclic form of a monosaccharide and, by extension, of a lower oligosaccharide. Glycosylation of proteins in a cell is most commonly an enzymatic process and the enzymatic machineries from different organisms that are responsible for glycosylation may differ in their preference for glycosylation sites. As a consequence, the glycosylated residues and the nature of the glycosyl group may vary between proteins produced in different host organisms. Accordingly, a “glycosylation pattern” as used herein, refers to a specific set of glycan structures on a protein that is mainly determined by the production host.

Protein glycosylation has a significant influence on the biological activity of a protein. Especially for therapeutic proteins, it is of great importance that the glycosylation pattern of the protein remains constant, to ensure consistent efficacy and compatibility of these proteins. In general, the glycosylation pattern of a protein highly depends on the host organism in which the protein has been produced. While variations in glycosylation patterns of proteins are frequently observed between different eukaryotic organisms, it is rather uncommon to observe protein glycosylation in proteins that have been produced in bacterial host organisms. Bacteria as production hosts have the advantage that bacterial cells can grow in significantly larger volumes and at higher cell densities than mammalian cells, which makes bacteria a preferred production host for proteins that do not require specific glycosylation patterns for their activity. In general, the protein according to the invention may be produced in any host organism. However, to allow high production levels, the protein according to the invention may be preferably produced in bacterial host organisms. The proteins variants of the present invention have been produced as non-glycosylated proteins in a bacterial production host. Thus, the protein according to the invention may not be glycosylated.

As described above, the four α-helices that form the bundle of four α-helices may be located on a single polypeptide chain or on two separate polypeptide chains. In a specific embodiment, the invention relates to a protein according to the invention, wherein the α-helices that form the bundle of four α-helices are located on a single polypeptide chain.

In a preferred embodiment, the invention relates to a G-CSF-like protein according to the invention, wherein the single polypeptide chain comprises a four-helix bundle arrangement.

A polypeptide chain is said to have a “four-helix bundle arrangement”, if all four α-helices that contribute to a bundle of four α-helices are located on said polypeptide chain. The protein variants Boskar_1-4 (SEQ ID NO:2-5), Moevan (SEQ ID NO:6) and Sohair (SEQ ID NO:14) as provided herein all comprise four α-helices that form the bundle of four α-helices on a single polypeptide. Thus, the respective protein variants, as well as G-CSF, are said to comprise a four-helix bundle arrangement.

In a more preferred embodiment, the invention relates to a G-CSF-like protein according to the invention, wherein the four-helix bundle arrangement has an up-down-up-down topology.

The four-helix bundle arrangement of human G-CSF has an up-up-down-down topology, meaning that α-helices A and B are pointing in an upward direction and α-helices C and D are pointing in a downward direction, when visualized in an N-to-C-terminal direction. This has the disadvantage that between α-helices A and B, a bundle-spanning amino acid linker is necessary to connect the C-terminal top end of α-helix A with the N-terminal bottom end of α-helix B. Similarly, a bundle-spanning amino acid linker is necessary to connect the C-terminal bottom end of α-helix C with the N-terminal top end of α-helix D.

In general, the four-helix bundle arrangement of the protein according to the invention may have any topology. However, it is preferred that the proteins according to the invention have significantly shorter amino acid linkers between contiguous bundle-forming α-helices that are located on the same polypeptide chain compared to G-CSF. To accommodate such short amino acid linkers in a four helix-bundle arrangement, the polypeptide chain of the protein according to the invention may have an up-down-up-down topology. An “up-down-up-down topology” as used herein is characterized in that the C-terminal top end of a first α-helix is connected to the N-terminal top end of the following α-helix, or that the C-terminal bottom end of a first α-helix is connected to the N-terminal bottom end of the following α-helix. Accordingly, the protein variants Boskar_1-4 (SEQ ID NO:2-5), Moevan (SEQ ID NO:6) and Sohair (SEQ ID NO:14) of the present invention all comprise a single polypeptide chain with a four-helix bundle arrangement and an up-down-up-down topology.

In certain embodiments, at least 50%, at least 55%, at least 60%, at least 65%, at least 70% or at least 80% of the amino acids in the G-CSF-like protein according to the invention are involved in the formation of α-helical structures, in particular in the formation of α-helical structures that contribute to the four-helix bundle.

The protein according to the invention may be characterized in that it comprises one or more of the features of the preceding claims in any combination. Preferably, the protein according to the invention may share some degree of amino acid sequence identity with the protein variants Boskar_4 (SEQ ID NO:5), Boskar_3 (SEQ ID NO:4), Boskar_2 (SEQ ID NO:3), Boskar_1 (SEQ ID NO:2), Moevan (SEQ ID NO:6) or Sohair (SEQ ID NO:14). Thus, in a preferred embodiment, the invention relates to a G-CSF-like protein according to the invention, wherein the single polypeptide chain comprises an amino acid sequence having at least 60% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In a more preferred embodiment, the invention relates to a G-CSF-like protein according to the invention, wherein the single polypeptide chain comprises an amino acid sequence having at least 70% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In an even more preferred embodiment, the invention relates to a G-CSF-like protein according to the invention, wherein the single polypeptide chain comprises an amino acid sequence having at least 80% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In an even more preferred embodiment, the invention relates to a G-CSF-like protein according to the invention, wherein the single polypeptide chain comprises an amino acid sequence having at least 90% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In an even more preferred embodiment, the invention relates to a G-CSF-like protein according to the invention, wherein the single polypeptide chain comprises an amino acid sequence having at least 95% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In an even more preferred embodiment, the invention relates to a G-CSF-like protein according to the invention, wherein the single polypeptide chain comprises an amino acid sequence having at least 96% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In an even more preferred embodiment, the invention relates to a G-CSF-like protein according to the invention, wherein the single polypeptide chain comprises an amino acid sequence having at least 97% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In an even more preferred embodiment, the invention relates to a G-CSF-like protein according to the invention, wherein the single polypeptide chain comprises an amino acid sequence having at least 98% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In an even more preferred embodiment, the invention relates to a G-CSF-like protein according to the invention, wherein the single polypeptide chain comprises an amino acid sequence having at least 99% amino acid sequence identity with an amino acid sequence selected from the group consisting of: SEQ ID NO:5, SEQ ID NO:4, SEQ ID NO:3, SEQ ID NO:2, SEQ ID NO:6 and SEQ ID NO:14. In a most preferred embodiment, the invention relates to a G-CSF-like protein according to the invention, wherein the single polypeptide chain comprises an amino acid sequence selected from the group consisting of: SEQ ID NO:5, SEQ ID NO:4, SEQ ID NO:3, SEQ ID NO:2, SEQ ID NO:6 and SEQ ID NO:14.

In an alternative embodiment, the invention relates to a G-CSF-like protein according to the invention, wherein the α-helices that form the bundle of four α-helices are located on two separate polypeptide chains.

That is, the four α-helices that form the bundle of four α-helices may be located on two separate polypeptide chains. The G-CSF-like protein according to the invention may comprise one polypeptide chain that contributes one α-helix to the bundle of four α-helices and one polypeptide chain that contributes three α-helices to the bundle of four α-helices. Alternatively, the G-CSF-like protein according to the invention may comprise two polypeptide chains that contribute two α-helices to the bundle of four α-helices, respectively.

The protein variants Disohair_2 (SEQ ID NO:19) and Disohair_1 (SEQ ID NO:18) of the present invention comprise two polypeptide chains and each of the polypeptide chains contributes two α-helices to the bundle of four α-helices. Thus, in a preferred embodiment, the invention relates to a G-CSF-like protein according to the invention, wherein each of the two polypeptide chains contributes two α-helices to the bundle of four α-helices.

In general, polypeptide chains that contribute two α-helices to the bundle of four α-helices may comprise any structural motif. One of the simplest structural motifs that comprise two α-helices is a helical-hairpin motif. Thus, in a more preferred embodiment, the invention relates to a G-CSF-like protein according to the invention, wherein each of the two polypeptide chains comprises a helical-hairpin motif. A “helical-hairpin motif” as used herein, refers to a protein motif that comprises two interacting helices that are connected by a turn or a short loop.

In an even more preferred embodiment, the invention relates to a G-CSF-like protein according to the invention, wherein the two polypeptide chains form a dimer.

The term “dimer” as used herein, refers to a macromolecular complex consisting of two subunits called monomers. The term “complex” or “macromolecular complex” as used herein in reference to a protein, relates to a group of two or more associated polypeptide chains. Different polypeptide chains may have different functions. The polypeptide chains in a complex are typically connected by non-covalent bonds, such as electrostatic interaction, van-der-Waals forces, hydrogen bonds, 7-effects and hydrophobic effects.

In case of proteins, a “dimer” refers to a protein or part of a protein that consists of two polypeptide chains that form a complex. That is, the protein according to the invention may be a macromolecular complex that comprises two polypeptide chains. The two polypeptide chains that form the protein according to the invention may be identical or may differ in their amino acid sequence. Accordingly, the G-CSF-like protein according to the invention may be a homodimer, wherein the two polypeptide chains are identical in sequence, or may be a heterodimer, wherein the two polypeptide chains are not identical in sequence.

The G-CSF-like protein according to the invention may be characterized in that it comprises one or more of the features of the preceding claims in any combination. Preferably, the G-CSF-like protein according to the invention may share some degree of amino acid sequence identity with the protein variants Disohair_2 (SEQ ID NO:19) and Disohair_1 (SEQ ID NO:18). Thus, in a preferred embodiment, the invention relates to a G-CSF-like protein according to the invention, wherein both polypeptide chains comprise an amino acid sequence having at least 60% amino acid sequence identity with an amino acid sequence selected from the group consisting of: SEQ ID NO:19 and SEQ ID NO:18. In a more preferred embodiment, the invention relates to a G-CSF-like protein according to the invention, wherein both polypeptide chains comprise an amino acid sequence having at least 70% amino acid sequence identity with an amino acid sequence selected from the group consisting of: SEQ ID NO:19 and SEQ ID NO:18. In an even more preferred embodiment, the invention relates to a G-CSF-like protein according to the invention, wherein both polypeptide chains comprise an amino acid sequence having at least 80% amino acid sequence identity with an amino acid sequence selected from the group consisting of: SEQ ID NO:19 and SEQ ID NO:18. In an even more preferred embodiment, the invention relates to a G-CSF-like protein according to the invention, wherein both polypeptide chains comprise an amino acid sequence having at least 90% amino acid sequence identity with an amino acid sequence selected from the group consisting of: SEQ ID NO:19 and SEQ ID NO:18. In an even more preferred embodiment, the invention relates to a G-CSF-like protein according to the invention, wherein both polypeptide chains comprise an amino acid sequence having at least 95% amino acid sequence identity with an amino acid sequence selected from the group consisting of: SEQ ID NO:19 and SEQ ID NO:18. In an even more preferred embodiment, the invention relates to a G-CSF-like protein according to the invention, wherein both polypeptide chains comprise an amino acid sequence having at least 96% amino acid sequence identity with an amino acid sequence selected from the group consisting of: SEQ ID NO:19 and SEQ ID NO:18. In an even more preferred embodiment, the invention relates to a G-CSF-like protein according to the invention, wherein both polypeptide chains comprise an amino acid sequence having at least 97% amino acid sequence identity with an amino acid sequence selected from the group consisting of: SEQ ID NO:19 and SEQ ID NO:18. In an even more preferred embodiment, the invention relates to a G-CSF-like protein according to the invention, wherein both polypeptide chains comprise an amino acid sequence having at least 98% amino acid sequence identity with an amino acid sequence selected from the group consisting of: SEQ ID NO:19 and SEQ ID NO:18. In an even more preferred embodiment, the invention relates to a G-CSF-like protein according to the invention, wherein both polypeptide chains comprise an amino acid sequence having at least 99% amino acid sequence identity with an amino acid sequence selected from the group consisting of: SEQ ID NO:19 and SEQ ID NO:18. In a most preferred embodiment, the invention relates to a G-CSF-like protein according to the invention, wherein both polypeptide chains comprise an amino acid sequence selected from the group consisting of: SEQ ID NO:19 and SEQ ID NO:18.

Certain preferred aspects provided herein are based, in part, on the development of the protein variant Boskar_4 (SEQ ID NO:5), which has G-CSF-like activity.

Accordingly, in one aspect the invention relates to a protein comprising or consisting of an amino acid sequence having at least 60%, 70%, 80%, 90%, 95%, 96%, 97%, 98%, 99%, or 100% amino acid sequence identity with the amino acid sequence of SEQ ID NO:5, wherein the protein has G-CSF-like activity. Preferably, said protein comprises said amino acid sequence in a single polypeptide chain.

Preferably, the invention discloses a protein comprising or consisting of a single polypeptide chain with an amino acid sequence having at least 60%, 70%, 80%, 90%, 95%, 96%, 97%, 98% or 99%, amino acid sequence identity with the amino acid sequence of SEQ ID NO:5, wherein the protein has G-CSF-like activity, wherein at least one of the amino acid residues Alanine 6, Tyrosine 11, Alanine 15, Lysine 22, Methionine 42, Methionine 49, Alanine 52, Glycine 56, Leucine 57, Aspartate 58, Serine 59, Lysine 91, Glycine 92, Asparagine 93, Aspartate 94 and Glutamine 115 in the amino acid sequence shown in SEQ ID NO:5 is substituted.

Amino acid residue Alanine 6 of SEQ ID NO:5 may preferably be substituted with a valine or glutamate residue. Amino acid residue Tyrosine 11 of SEQ ID NO:5 may preferably be substituted with a methionine residue. Amino acid residue Alanine 15 of SEQ ID NO:5 may preferably be substituted with a glutamine residue. Amino acid residue Lysine 22 of SEQ ID NO:5 may preferably be substituted with a glutamine residue. Amino acid residue Methionine 42 of SEQ ID NO:5 may preferably be substituted with a valine residue. Amino acid residue Methionine 49 of SEQ ID NO:5 may preferably be substituted with a isoleucine or leucine residue. Amino acid residue Alanine 52 of SEQ ID NO:5 may preferably be substituted with a methionine residue. Amino acid residue Glycine 56 of SEQ ID NO:5 may preferably be substituted with an asparagine or lysine residue. Amino acid residue Leucine 57 of SEQ ID NO:5 may preferably be substituted with a proline or lysine residue. Amino acid residue Aspartate 58 of SEQ ID NO:5 may preferably be substituted with a serine, glycine or threonine residue. Amino acid residue Serine 59 of SEQ ID NO:5 may preferably be substituted with an aspartate, proline or asparagine residue. Amino acid residue Lysine 91 of SEQ ID NO:5 may preferably be substituted with a proline or threonine residue. Amino acid residue Glycine 92 of SEQ ID NO:5 may preferably be substituted with an asparagine, serine or glycine residue. Amino acid Asparagine 93 of SEQ ID NO:5 may preferably be substituted with a serine or threonine residue. Amino acid residue Aspartate 94 of SEQ ID NO:5 may preferably be substituted with a glutamine residue. Amino acid residue Glutamine 115 of SEQ ID NO:5 may preferably be substituted with a glutamate residue.

In particular, the invention also provides a protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, SEQ ID NO:4, SEQ ID NO:3 or SEQ ID NO:2, wherein the protein has G-CSF-like activity. Preferably, said protein comprises said amino acid sequence in a single polypeptide chain.

In one embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein comprises: a) a bundle of four α-helices; and b) three amino acid linkers that connect contiguous bundle-forming α-helices, wherein each amino acid linker has a length between 2 and 20 amino acids.

In one embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein comprises: a) a bundle of four α-helices; and b) three amino acid linkers that connect contiguous bundle-forming α-helices, wherein each amino acid linker has a length between 2 and 15 amino acids.

In one embodiment, the present invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein has a melting temperature of at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C. or at least 95° C.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein comprises one or more G-CSF receptor binding sites.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein each G-CSF receptor binding site individually comprises six to eight amino acid residues having an identical structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

In certain embodiments, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein binds to G-CSF-R with a binding affinity of less than 1 mM, less than 900 μM, less than 800 μM, less than 700 μM, less than 600 μM, less than 500 μM, less than 400 μM, less than 300 μM, less than 200 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM or less than 1 μM.

Alternatively, in certain embodiments, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein binds to G-CSF-R with a binding affinity ranging from 0.1 nM to 1 mM, from 0.1 nM to 500 μM, ranging from 0.1 nM to 100 μM, ranging from 0.1 nM to 50 μM, ranging from 0.1 nM to 25 μM, ranging from 0.1 nM to 10 μM, ranging from 0.5 nM to 10 μM or ranging from 1 nM to 10 μM.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the G-CSF-like activity comprises at least one, preferably at least two, more preferably at least three, most preferably all of the following activities: (i) induction of granulocytic differentiation of HSPCs; (ii) induction of the formation of myeloid colony-forming units from HSPCs; (iii) induction of the proliferation of NFS-60 cells; and/or (iv) activation of the downstream signaling pathways MAPK/ERK and/or JAK/STAT.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein induces the proliferation of NFS-60 cells. In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein induces the proliferation of NFS-60 cells in a culture at a half maximal effective concentration (EC50) of less than 100 μg/mL, preferably less than 50 μg/mL, preferably less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, preferably less than 1 μg/mL, preferably less than 0.75 μg/mL, preferably less than 0.5 μg/mL, preferably less than 0.25 μg/mL or preferably less than 0.1 μg/mL.

In another embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein induces the proliferation and/or differentiation of cells comprising one or more G-CSF receptor on the cell surface.

In another embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.

In another embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the calculated contact order number of said protein is lower than the calculated contact order number of human G-CSF (SEQ ID NO:1).

In another embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein has a molecular mass between 12 and 15 kDa.

In another embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein comprises no disulfide bonds.

In another embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:5, wherein the protein is not glycosylated.

Certain aspects provided herein are based, in part, on the development of the protein variant Moevan (SEQ ID NO:6), which has G-CSF-like activity.

Accordingly, in one aspect the invention relates to a protein comprising or consisting of an amino acid sequence having at least 60%, 70%, 80%, 90%, 95%, 96%, 97%, 98%, 99%, or 100% amino acid sequence identity with the amino acid sequence of SEQ ID NO:6, wherein the protein has G-CSF-like activity. Preferably, said protein comprises said amino acid sequence in a single polypeptide chain.

Preferably, the invention discloses a protein comprising or consisting of a single polypeptide chain with an amino acid sequence having at least 60%, 70%, 80%, 90%, 95%, 96%, 97%, 98% or 99% amino acid sequence identity with the amino acid sequence of SEQ ID NO:6, wherein the protein has G-CSF-like activity, wherein at least one of the amino acid residues Serine 11, Leucine 14, Alanine 25, Serine 31, Glutamate 32, Aspartate 40, Threonine 41, Valine 50, Threonine 51, Glutamine 55, Glutamate 61, Phenylalanine 64, Glycine 65, Arginine 66, Asparagine 67, Arginine 68, Aspartate 82, Leucine 86, Aspartate 87, Aspartate 90, Leucine 93, Alanine 94, Lysine 95, Glutamate 96, Lysine 97, Lysine 98 and Asparagine 104 in the amino acid sequence shown in SEQ ID NO:6 is deleted or substituted.

Amino acid residue Serine 11 of SEQ ID NO:6 may preferably be substituted with a lysine residue. Amino acid residue Lysine 14 of SEQ ID NO:6 may preferably be substituted with a isoleucine, arginine or tryptophan residue. Amino acid residue Alanine 25 of SEQ ID NO:6 may preferably be substituted with a arginine, glutamine or glutamate residue. Amino acid residue Serine 31 of SEQ ID NO:6 may preferably be substituted with a valine residue. Amino acid residue Glutamate 32 of SEQ ID NO:6 may preferably be substituted with a glutamine residue. Amino acid residue Aspartate 40 of SEQ ID NO:6 may preferably be substituted with a glutamate residue. Amino acid residue Threonine 41 of SEQ ID NO:6 may preferably be substituted with a lysine or arginine residue. Amino acid residue Valine 50 of SEQ ID NO:6 may preferably be substituted with an isoleucine residue. Amino acid residue Threonine 51 of SEQ ID NO:6 may preferably be substituted with a serine, glutamate, glutamine or isoleucine residue. Amino acid residue Glutamine 55 of SEQ ID NO:6 may preferably be substituted with a serine, glutamate, asparagine or arginine residue. Amino acid residue Glutamate 61 of SEQ ID NO:6 may preferably be substituted with a isoleucine residue. Amino acid residue Phenylalanine 64 of SEQ ID NO:6 may be deleted. Amino acid Glycine 64 of SEQ ID NO:6 may be deleted. Amino acid residue Arginine 66 of SEQ ID NO:6 may preferably be substituted with a leucine, asparagine or lysine residue. Amino acid residue Asparagine 67 of SEQ ID NO:6 may preferably be substituted with a leucine or threonine residue. Amino acid residue Arginine 68 of SEQ ID NO:6 may preferably be substituted with a aspartate or serine residue. Amino acid residue Aspartate 82 of SEQ ID NO:6 may preferably be substituted with a glutamate residue. Amino acid residue Leucine 86 of SEQ ID NO:6 may preferably be substituted with a lysine residue. Amino acid residue Aspartate 87 of SEQ ID NO:6 may preferably be substituted with a glutamate residue. Amino acid residue Aspartate 90 of SEQ ID NO:6 may preferably be substituted with a glutamate residue. Amino acid Leucine 93 of SEQ ID NO:6 may be deleted. Amino acid residue Alanine 94 of SEQ ID NO:6 may preferably be substituted with a lysine residue. Amino acid residue Lysine 95 of SEQ ID NO:6 may preferably be substituted with a serine or glutamate residue. Amino acid residue Glutamate 96 of SEQ ID NO:6 may preferably be substituted with a lysine, serine or glycine residue. Amino acid residue Lysine 97 of SEQ ID NO:6 may preferably be substituted with a proline, leucine or serine residue. Amino acid residue Lysine 98 of SEQ ID NO:6 may preferably be substituted with a serine or asparagine residue. Amino acid residue Asparagine 104 of SEQ ID NO:6 may preferably be substituted with a lysine residue.

In particular, the invention also provides a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, SEQ ID NO: 7, SEQ ID NO: 8, SEQ ID NO:9, SEQ ID NO: 10, SEQ ID NO: 11, SEQ ID NO: 12, SEQ ID NO:13, SEQ ID NO:20, SEQ ID NO:21 or SEQ ID NO:22; wherein the protein has G-CSF-like activity. Preferably, said protein comprises said amino acid sequence in a single polypeptide chain.

In one embodiment, the invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein comprises: a) a bundle of four α-helices; and b) three amino acid linkers that connect contiguous bundle-forming α-helices, wherein each amino acid linker has a length between 2 and 20 amino acids.

In one embodiment, the present invention relates to a G-CSF-like protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein has a melting temperature of at least 74° C.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein comprises one or more G-CSF receptor binding sites.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein each G-CSF receptor binding site individually comprises six to eight amino acid residues having an identical structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

In certain embodiments, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein binds to G-CSF-R with a binding affinity of less than 1 mM, less than 900 μM, less than 800 μM, less than 700 μM, less than 600 μM, less than 500 μM, less than 400 μM, less than 300 μM, less than 200 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM or less than 1 μM.

Alternatively, in certain embodiments, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein binds to G-CSF-R with a binding affinity ranging from 0.1 nM to 1 mM, from 0.1 nM to 500 μM, ranging from 0.1 nM to 100 μM, ranging from 0.1 nM to 50 μM, ranging from 0.1 nM to 25 μM, ranging from 0.1 nM to 10 μM, ranging from 0.5 nM to 10 μM or ranging from 1 nM to 10 μM.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the G-CSF-like activity comprises at least one, preferably at least two, more preferably at least three, most preferably all of the following activities: (i) induction of granulocytic differentiation of HSPCs; (ii) induction of the formation of myeloid colony-forming units from HSPCs; (iii) induction of the proliferation of NFS-60 cells; and/or (iv) activation of the downstream signaling pathways MAPK/ERK and/or JAK/STAT.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein induces the proliferation of NFS-60 cells.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein induces the proliferation of NFS-60 cells. In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein induces the proliferation of NFS-60 cells in a culture at a half maximal effective concentration (EC50) of less than 100 μg/mL, preferably less than 50 μg/mL, preferably less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, preferably less than 1 μg/mL, preferably less than 0.75 μg/mL, preferably less than 0.5 μg/mL, preferably less than 0.25 μg/mL or preferably less than 0.1 μg/mL.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein induces the proliferation and/or differentiation of cells comprising one or more G-CSF receptor on the cell surface.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the calculated contact order number of said protein is lower than the calculated contact order number of human G-CSF (SEQ ID NO:1).

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein has a molecular mass between 12 and 15 kDa.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein comprises no disulfide bonds.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:6, wherein the protein is not glycosylated.

Certain aspects provided herein are based, in part, on the development of the protein variant Sohair (SEQ ID NO:14), which has G-CSF-like activity.

Accordingly, in one aspect the invention relates to a protein comprising or consisting of an amino acid sequence having at least 60%, 70%, 80%, 90%, 95%, 96%, 97%, 98%, 99%, or 100% amino acid sequence identity with the amino acid sequence of SEQ ID NO:14, wherein the protein has G-CSF-like activity. Preferably, said protein comprises said amino acid sequence in a single polypeptide chain.

Preferably, the invention discloses a protein comprising or consisting of a single polypeptide chain with an amino acid sequence having at least 60%, 70%, 80%, 90%, 95%, 96%, 97%, 98% or 99% 100% amino acid sequence identity with the amino acid sequence of SEQ ID NO:14, wherein the protein has G-CSF-like activity, wherein at least one of the amino acid residues Glutamate 16, Methionine 24, Alanine 30, Asparagine 46, Leucine 49, Glutamine 60, Aspartate 91, Glutamate 94, Lysine 97, Alanine 102, Glutamate 104, Arginine 105, Arginine 108, Aspartate 124, Arginine 127, Glutamate 128, Glutamate 131, Glutamate 134, Glutamate 135, Arginine 138, Arginine 141 or Arginine 142 in the amino acid sequence shown in SEQ ID NO:14 is substituted.

Amino acid residue Glutamate 16 of SEQ ID NO:14 may preferably be substituted with a leucine, isoleucine, lysine or tryptophan residue. Amino acid residue Methionine 24 of SEQ ID NO:14 may preferably be substituted with a glutamine residue. Amino acid residue Alanine 30 of SEQ ID NO:14 may preferably be substituted with a glutamate. Amino acid residue Asparagine 46 of SEQ ID NO:14 may preferably be substituted with a glutamine, isoleucine or lysine residue. Amino acid residue Leucine 49 of SEQ ID NO:14 may preferably be substituted with a glutamine, tryptophan or isoleucine residue. Amino acid residue Glutamine 60 of SEQ ID NO:14 may preferably be substituted with a leucine, histidine, tyrosine, glutamate or alanine residue. Amino acid residue Aspartate 91 of SEQ ID NO:14 may preferably be substituted with a lysine residue. Amino acid residue Glutamate 94 of SEQ ID NO:14 may preferably be substituted with a leucine, lysine, isoleucine or tryptophan residue. Amino acid residue Lysine 97 of SEQ ID NO:14 may preferably be substituted with a leucine, glutamine, tyrosine or tryptophan residue. Amino acid residue Alanine 102 of SEQ ID NO:14 may preferably be substituted with a glutamine residue. Amino acid residue Glutamate 104 of SEQ ID NO:14 may preferably be substituted with a arginine residue. Amino acid residue Arginine 105 of SEQ ID NO:14 may preferably be substituted with a lysine residue. Amino acid residue Arginine 108 of SEQ ID NO:14 may preferably be substituted with a glutamate residue. Amino acid residue Aspartate 124 of SEQ ID NO:14 may preferably be substituted with a glutamine, isoleucine or lysine residue. Amino acid Arginine 127 of SEQ ID NO:14 may preferably be substituted with a glutamine, leucine, tryptophan or isoleucine residue. Amino acid residue Glutamate 128 of SEQ ID NO:14 may preferably be substituted with a aspartate residue. Amino acid residue Glutamate 131 of SEQ ID NO:14 may preferably be substituted with a aspartate residue. Amino acid residue Glutamate 134 of SEQ ID NO:14 may preferably be substituted with a threonine residue. Amino acid residue Glutamate 135 of SEQ ID NO:14 may preferably be substituted with a threonine residue. Amino acid residue Arginine 138 of SEQ ID NO:14 may preferably be substituted with a leucine, glutamate, histidine, tyrosine or alanine residue. Amino acid residue Arginine 141 of SEQ ID NO:14 may preferably be substituted with a glutamate residue. Amino acid residue Arginine 142 of SEQ ID NO:14 may preferably be substituted with a glutamate residue.

In particular, the invention also provides a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, SEQ ID NO: 15, SEQ ID NO: 16, SEQ ID NO:17, SEQ ID NO: 23, SEQ ID NO: 24 or SEQ ID NO: 25 wherein the protein has G-CSF-like activity. Preferably, said protein comprises said amino acid sequence in a single polypeptide chain.

In one embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein comprises: a) a bundle of four α-helices; and b) three amino acid linkers that connect contiguous bundle-forming α-helices, wherein each amino acid linker has a length between 2 and 20 amino acids.

In one embodiment, the present invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein has a melting temperature of at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C. or at least 95° C.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein comprises one or more G-CSF receptor binding sites.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein each G-CSF receptor binding site individually comprises six to eight amino acid residues having an identical structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

In certain embodiments, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein binds to G-CSF-R with a binding affinity of less than 1 mM, less than 900 μM, less than 800 μM, less than 700 μM, less than 600 μM, less than 500 μM, less than 400 μM, less than 300 μM, less than 200 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM or less than 1 μM.

Alternatively, in certain embodiments, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein binds to G-CSF-R with a binding affinity ranging from 0.1 nM to 1 mM, from 0.1 nM to 500 μM, ranging from 0.1 nM to 100 μM, ranging from 0.1 nM to 50 μM, ranging from 0.1 nM to 25 μM, ranging from 0.1 nM to 10 μM, ranging from 0.5 nM to 10 μM or ranging from 1 nM to 10 μM.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the G-CSF-like activity comprises at least one, preferably at least two, more preferably at least three, most preferably all of the following activities: (i) induction of granulocytic differentiation of HSPCs; (ii) induction of the formation of myeloid colony-forming units from HSPCs; (iii) induction of the proliferation of NFS-60 cells; and/or (iv) activation of the downstream signaling pathways MAPK/ERK and/or JAK/STAT.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein induces the proliferation of NFS-60 cells.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein induces the proliferation of NFS-60 cells. In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein induces the proliferation of NFS-60 cells in a culture at a half maximal effective concentration (EC50) of less than 100 μg/mL, preferably less than 50 μg/mL, preferably less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, preferably less than 1 μg/mL, preferably less than 0.75 μg/mL, preferably less than 0.5 μg/mL, preferably less than 0.25 μg/mL or preferably less than 0.1 μg/mL.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein induces the proliferation and/or differentiation of cells comprising one or more G-CSF receptor on the cell surface.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the calculated contact order number of said protein is lower than the calculated contact order number of human G-CSF (SEQ ID NO:1).

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein has a molecular mass between 16 and 18 kDa.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein comprises no disulfide bonds.

In another embodiment, the invention relates to a protein comprising or consisting of an amino acid sequence having at least 60%, 70%, 80%, 90%, 95%, 96%, 97%, 98%, 99% or 100% amino acid sequence identity with the amino acid sequence of SEQ ID NO:14, wherein the protein is not glycosylated.

Certain aspects provided herein are based, in part, on the development of the protein variant Disohair_2 (SEQ ID NO:19), which has G-CSF-like activity.

Accordingly, in one aspect the invention relates to a protein comprising an amino acid sequence having at least 60%, 70%, 80%, 90%, 95%, 96%, 97%, 98%, 99%, or 100% amino acid sequence identity with the amino acid sequence of SEQ ID NO:19, wherein the protein has G-CSF-like activity. Preferably, said protein comprises two polypeptide chains, wherein each polypeptide chain comprises said amino acid sequence. More preferably, the two polypeptide chains of the protein comprise identical amino acid sequences.

Preferably, the invention discloses a protein comprising a polypeptide chain with an amino acid sequence having at least 60%, 70%, 80%, 90%, 95%, 96%, 97%, 98% or 99% amino acid sequence identity with the amino acid sequence of SEQ ID NO:19, wherein the protein has G-CSF-like activity, wherein at least one of the amino acid residues Glutamate 16, Glutamine 24, Alanine 30, Asparagine 46, Leucine 49 or Glutamine 60 in the amino acid sequence shown in SEQ ID NO:19 is substituted.

Amino acid residue Glutamate 16 of SEQ ID NO:19 may preferably be substituted with a leucine, lysine or tryptophan residue. Amino acid residue Glutamine 24 of SEQ ID NO:19 may preferably be substituted with a methionine residue. Amino acid residue Alanine 30 of SEQ ID NO:19 may preferably be substituted with a glutamate. Amino acid residue Asparagine 46 of SEQ ID NO:19 may preferably be substituted with a glutamine or lysine residue. Amino acid residue Leucine 49 of SEQ ID NO:19 may preferably be substituted with a glutamine or isoleucine residue. Amino acid residue Glutamine 60 of SEQ ID NO:19 may preferably be substituted with a leucine glutamate or alanine residue.

In particular, the invention also provides a protein comprising an amino acid sequence having at least 60%, 70%, 80%, 90%, 95%, 96%, 97%, 98%, 99% or 100% amino acid sequence identity with the amino acid sequence of SEQ ID NO:19 or SEQ ID NO:18, wherein the protein has G-CSF-like activity.

Preferably, the protein comprises two polypeptide chains, wherein both polypeptide chains comprise or consist of amino acid sequences having at least 60%, 70%, 80%, 90%, 95%, 96%, 97%, 98%, 99% or 100% amino acid sequence identity with the amino acid sequences of SEQ ID NO:19 and/or SEQ ID NO:18. More preferably, the two polypeptide chains of the protein comprise identical amino acid sequences.

In one embodiment, the invention relates to a protein according to the invention, wherein the protein comprises: a) two polypeptide chains, wherein each polypeptide chain independently comprises an amino acid sequence having at least 60%, 70%, 80%, 90%, 95%, 96%, 97%, 98%, 99% or 100% amino acid sequence identity with the amino acid sequence of SEQ ID NO:19; (b) a bundle of four α-helices; and c) two amino acid linkers that connect contiguous bundle-forming α-helices that are located on the same polypeptide chain, wherein each amino acid linker has a length between 2 and 20 amino acids. Preferably, the two polypeptide chains of the protein comprise identical amino acid sequences.

In one embodiment, the present invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein has a melting temperature of at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C. or at least 95° C.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein comprises one or more G-CSF receptor binding sites.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein each G-CSF receptor binding site individually comprises six to eight amino acid residues having an identical structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

In certain embodiments, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein binds to G-CSF-R with a binding affinity of less than 1 mM, less than 900 μM, less than 800 μM, less than 700 μM, less than 600 μM, less than 500 μM, less than 400 μM, less than 300 μM, less than 200 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM or less than 1 μM.

Alternatively, in certain embodiments, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein binds to G-CSF-R with a binding affinity ranging from 0.1 nM to 1 mM, from 0.1 nM to 500 μM, ranging from 0.1 nM to 100 μM, ranging from 0.1 nM to 50 μM, ranging from 0.1 nM to 25 μM, ranging from 0.1 nM to 10 μM, ranging from 0.5 nM to 10 μM or ranging from 1 nM to 10 μM.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the G-CSF-like activity comprises at least one, preferably at least two, more preferably at least three, most preferably all of the following activities: (i) induction of granulocytic differentiation of HSPCs; (ii) induction of the formation of myeloid colony-forming units from HSPCs; (iii) induction of the proliferation of NFS-60 cells; and/or (iv) activation of the downstream signaling pathways MAPK/ERK and/or JAK/STAT.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein induces the proliferation of NFS-60 cells.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein induces the proliferation of NFS-60 cells. In another embodiment, the invention relates to a protein according to the invention, wherein the protein induces the proliferation of NFS-60 cells in a culture at a half maximal effective concentration (EC50) of less than 100 μg/mL, preferably less than 50 μg/mL, preferably less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, preferably less than 1 μg/mL, preferably less than 0.75 μg/mL, preferably less than 0.5 μg/mL, preferably less than 0.25 μg/mL or preferably less than 0.1 μg/mL.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein induces the proliferation and/or differentiation of cells comprising one or more G-CSF receptor on the cell surface.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the calculated contact order number of said protein is lower than the calculated contact order number of human G-CSF (SEQ ID NO:1).

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein has a molecular mass between 16 and 18 kDa.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein comprises no disulfide bonds.

In another embodiment, the invention relates to a protein comprising an amino acid sequence having at least 60%, 70%, 80%, 90%, 95%, 96%, 97%, 98%, 99% or 100% amino acid sequence identity with the amino acid sequence of SEQ ID NO:19, wherein the protein is not glycosylated.

Certain aspects provided herein are based, in part, on the development of the protein variant bika1 (SEQ ID NO:32), which has G-CSF-like activity.

Accordingly, in one aspect the invention relates to a protein comprising an amino acid sequence having at least 60%, 70%, 80%, 90%, 95%, 96%, 97%, 98%, 99%, or 100% amino acid sequence identity with the amino acid sequence of SEQ ID NO:32, wherein the protein has G-CSF-like activity. Preferably, said protein comprises two polypeptide chains, wherein each polypeptide chain comprises said amino acid sequence. More preferably, the two polypeptide chains of the protein comprise identical amino acid sequences.

Preferably, the invention discloses a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein has G-CSF-like activity, wherein the amino acid residue Alanine 44 in the amino acid sequence shown in SEQ ID NO:32 is substituted. Amino acid residue Alanine 44 of SEQ ID NO:32 may preferably be substituted with a leucine residue.

In particular, the invention also provides a protein comprising an amino acid sequence having at least 60%, 70%, 80%, 90%, 95%, 96%, 97%, 98%, 99% or 100% amino acid sequence identity with the amino acid sequence of SEQ ID NO:32 or SEQ ID NO:33, wherein the protein has G-CSF-like activity.

Preferably, the protein comprises two polypeptide chains, wherein both polypeptide chains comprise or consist of amino acid sequences having at least 60%, 70%, 80%, 90%, 95%, 96%, 97%, 98%, 99% or 100% amino acid sequence identity with the amino acid sequences of SEQ ID NO:32 and/or SEQ ID NO:33. More preferably, the two polypeptide chains of the protein comprise identical amino acid sequences.

In one embodiment, the invention relates to a protein according to the invention, wherein the protein comprises: a) two polypeptide chains, wherein each polypeptide chain independently comprises an amino acid sequence having at least 60%, 70%, 80%, 90%, 95%, 96%, 97%, 98%, 99% or 100% amino acid sequence identity with the amino acid sequence of SEQ ID NO:32; (b) a bundle of four α-helices; and c) two amino acid linkers that connect contiguous bundle-forming α-helices that are located on the same polypeptide chain, wherein each amino acid linker has a length between 2 and 20 amino acids. Preferably, the two polypeptide chains of the protein comprise identical amino acid sequences.

In one embodiment, the present invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein has a melting temperature of at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C. or at least 95° C.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein comprises one or more G-CSF receptor binding sites.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein each G-CSF receptor binding site individually comprises six to eight amino acid residues having an identical structure and a similar special orientation towards each other as the amino acid residues Lysine 16, Glutamate 19, Glutamine 20, Arginine 22, Lysine 23, Aspartate 27, Aspartate 109, and Aspartate 112 of human G-CSF.

In certain embodiments, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein binds to G-CSF-R with a binding affinity of less than 1 mM, less than 900 μM, less than 800 μM, less than 700 μM, less than 600 μM, less than 500 μM, less than 400 μM, less than 300 μM, less than 200 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM or less than 1 μM.

Alternatively, in certain embodiments, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein binds to G-CSF-R with a binding affinity ranging from 0.1 nM to 1 mM, from 0.1 nM to 500 μM, ranging from 0.1 nM to 100 μM, ranging from 0.1 nM to 50 μM, ranging from 0.1 nM to 25 μM, ranging from 0.1 nM to 10 μM, ranging from 0.5 nM to 10 μM or ranging from 1 nM to 10 μM.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the G-CSF-like activity comprises at least one, preferably at least two, more preferably at least three, most preferably all of the following activities: (i) induction of granulocytic differentiation of HSPCs; (ii) induction of the formation of myeloid colony-forming units from HSPCs; (iii) induction of the proliferation of NFS-60 cells; and/or (iv) activation of the downstream signaling pathways MAPK/ERK and/or JAK/STAT.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein induces the proliferation of NFS-60 cells.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein induces the proliferation of NFS-60 cells. In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein induces the proliferation of NFS-60 cells in a culture at a half maximal effective concentration (EC50) of less than 100 μg/mL, preferably less than 50 μg/mL, preferably less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, preferably less than 1 μg/mL, preferably less than 0.75 μg/mL, preferably less than 0.5 μg/mL, preferably less than 0.25 μg/mL or preferably less than 0.1 μg/mL.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein induces the proliferation and/or differentiation of cells comprising one or more G-CSF receptor on the cell surface.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the calculated contact order number of said protein is lower than the calculated contact order number of human G-CSF (SEQ ID NO:1).

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein has a molecular mass between 14 and 18 kDa.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein comprises no disulfide bonds.

In another embodiment, the invention relates to a protein comprising a polypeptide chain with an amino acid sequence having at least 60%, 70%, 80%, 90%, 95%, 96%, 97%, 98% or 99% amino acid sequence identity with the amino acid sequence of SEQ ID NO:32, wherein the protein is not glycosylated.

In another aspect, the invention relates to a fusion protein comprising a first protein domain and a second protein domain, wherein the first protein domain and/or the second protein domain is a protein according to the invention.

That is, the protein designs of the present invention may be comprised in a fusion protein. The protein designs of the invention may be fused to any fusion partner, provided that the fusion partner does not negatively impact the stability or the biological activity of the protein design comprised in the fusion protein. Preferably, the fusion protein has similar or higher thermal stability compared the protein design comprised in the fusion protein.

The term “fusion protein”, as used herein, refers to a hybrid polypeptide that comprises protein domains from at least two different proteins. Within the present invention, at least one of the protein domains comprised in the fusion protein is derived from one of the protein designs disclosed herein.

In certain embodiments, the fusion protein may comprise a protein design according to the invention and a protein domain that increases stability of the fusion protein, in particular the thermal stability of the fusion protein. Protein domains that can be fused to a protein to increase the thermal stability of said protein are known in the art.

In certain embodiments, the fusion protein may comprise a protein design according to the invention and a therapeutic protein.

In certain embodiments, two protein designs according to the invention may be comprised in a fusion protein. For example, it has been demonstrated by the inventors that fusing two copies of the protein designs Boskar_4 or Moevan results in fusion proteins with a higher biological activity in comparison to the single protein designs (Table 7). In addition, it has been demonstrated that a fusion protein comprising two copies of Moevan binds to G-CSF-R with a significantly increased affinity (Example 11). Interestingly, the fusion protein comprising two copies of Moevan binds to G-CSF-R with a similar affinity as G-CSF (Table 10).

In one embodiment, the invention relates to the fusion protein according to the invention, wherein the first protein and the second protein are linked by a peptide linker.

That is, the protein domains comprised in the fusion protein are preferably fused with a peptide linker. In certain embodiments, the linker may be a linker that is rich in glycine and serine residues. In certain embodiments, at least 50%, at least 55%, at least 60%, at least 65%, at least 70%, at least 75%, at least 80%, at least 85%, at least 90% or at least 95% of the amino acid residues comprised in the linker are glycine or serine residues. In certain embodiments, the linker consists exclusively of glycine and serine residues.

Accordingly, in one embodiment, the invention relates to the fusion protein according to the invention, wherein the peptide linker is a glycine-serine linker.

In certain embodiments, the invention relates to the fusion protein according to the invention, wherein the linker has a length of 5 to 50 amino acid residues. In certain embodiments, the linker has a length of 5 to 40 amino acid residues. In certain embodiments, the linker has a length of 5 to 30 amino acid residues. In certain embodiments, the linker has a size of 5 to 25 amino acid residues.

In certain embodiments, the fusion protein comprises two identical protein designs according to the invention. However, it has to be noted that the two protein designs comprised in the fusion protein may have sequence variations.

In certain embodiments, the fusion protein comprises two copies of the protein design Boskar. That is, the fusion protein may comprise a first and a second protein domain, wherein each the first and the second protein domain comprise amino acid sequences that are independently selected from the group consisting of: SEQ ID NO:5, SEQ ID NO:3, SEQ ID NO:4 and SEQ ID NO:2. In certain embodiments, the fusion protein may comprise a first and a second protein domain, wherein both the first and the second protein domain comprise an amino acid sequence selected from the group consisting of: SEQ ID NO:5, SEQ ID NO:3, SEQ ID NO:4 and SEQ ID NO:2. In certain embodiments, the fusion protein comprises a first and a second protein domain, wherein both the first an the second protein domain comprise an amino acid sequence having at least 80%, at least 85%, at least 90% or at least 95% sequence identity to the amino acid sequence set forth in SEQ ID NO:5. In certain embodiments, the fusion protein according to the invention may comprise or consist of the amino acid sequence set forth in SEQ ID NO:26 or SEQ ID NO:27.

In certain embodiments, the fusion protein comprises two copies of the protein design Moevan. That is, the fusion protein may comprise a first and a second protein domain, wherein each the first and the second protein domain comprise amino acid sequences that are independently selected from the group consisting of: SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:20, SEQ ID NO:21 and SEQ ID NO:22. In certain embodiments, the fusion protein may comprise a first and a second protein domain, wherein both the first and the second protein domain comprise an amino acid sequence selected from the group consisting of: SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:20, SEQ ID NO:21 and SEQ ID NO:22. In certain embodiments, the fusion protein comprises a first and a second protein domain, wherein both the first an the second protein domain comprise an amino acid sequence having at least 80%, at least 85%, at least 90% or at least 95% sequence identity to the amino acid sequence set forth in SEQ ID NO:6. In certain embodiments, the fusion protein according to the invention may comprise or consist of the amino acid sequence set forth in SEQ ID NO:29 or SEQ ID NO:30.

In certain embodiments, the fusion protein comprises two copies of the protein design Sohair. That is, the fusion protein may comprise a first and a second protein domain, wherein each the first and the second protein domain comprise amino acid sequences that are independently selected from the group consisting of: SEQ ID NO:14, SEQ ID NO:15, SEQ ID NO:16, SEQ ID NO:17, SEQ ID NO:23, SEQ ID NO:24, SEQ ID NO:25 and SEQ ID NO:31. In certain embodiments, the fusion protein may comprise a first and a second protein domain, wherein both the first and the second protein domain comprise an amino acid sequence selected from the group consisting of: SEQ ID NO:14, SEQ ID NO:15, SEQ ID NO:16, SEQ ID NO:17, SEQ ID NO:23, SEQ ID NO:24, SEQ ID NO:25 and SEQ ID NO:31. In certain embodiments, the fusion protein comprises a first and a second protein domain, wherein both the first an the second protein domain comprise an amino acid sequence having at least 80%, at least 85%, at least 90% or at least 95% sequence identity to the amino acid sequence set forth in SEQ ID NO:14.

In certain embodiments, the fusion protein comprises two copies of the protein design DiSohair. That is, the fusion protein may comprise a first and a second protein domain, wherein each the first and the second protein domain comprise amino acid sequences that are independently selected from the group consisting of: SEQ ID NO:19 and SEQ ID NO:18. In certain embodiments, the fusion protein may comprise a first and a second protein domain, wherein both the first and the second protein domain comprise an amino acid sequence selected from the group consisting of: SEQ ID NO:19 and SEQ ID NO:18. In certain embodiments, the fusion protein comprises a first and a second protein domain, wherein both the first an the second protein domain comprise an amino acid sequence having at least 80%, at least 85%, at least 90% or at least 95% sequence identity to the amino acid sequence set forth in SEQ ID NO:19.

In certain embodiments, the fusion protein comprises two copies of the protein design bika. That is, the fusion protein may comprise a first and a second protein domain, wherein each the first and the second protein domain comprise amino acid sequences that are independently selected from the group consisting of: SEQ ID NO:32 and SEQ ID NO:33. In certain embodiments, the fusion protein may comprise a first and a second protein domain, wherein both the first and the second protein domain comprise an amino acid sequence selected from the group consisting of: SEQ ID NO:32 and SEQ ID NO:33. In certain embodiments, the fusion protein comprises a first and a second protein domain, wherein both the first an the second protein domain comprise an amino acid sequence having at least 80%, at least 85%, at least 90% or at least 95% sequence identity to the amino acid sequence set forth in SEQ ID NO:32.

In certain embodiments, the invention relates to the fusion protein according to the invention, wherein the first protein domain and the second protein domain comprise identical amino acid sequences.

In one embodiment, the present invention relates to the fusion protein according to the invention, wherein the fusion protein has a melting temperature (Tₘ) of at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C. or at least 95° C.

In another embodiment, the invention relates to the fusion protein according to the invention, wherein the fusion protein comprises one or more G-CSF receptor binding sites. In another embodiment, the invention relates to the fusion protein according to the invention, wherein the fusion protein comprises at least two G-CSF receptor binding sites. In another embodiment, the invention relates to the fusion protein according to the invention, wherein the fusion protein comprises four G-CSF receptor binding sites.

In certain embodiments, the invention relates to the fusion protein according to the invention, wherein the fusion protein binds to G-CSF-R with a binding affinity of less than 1 mM, less than 900 μM, less than 800 μM, less than 700 μM, less than 600 μM, less than 500 μM, less than 400 μM, less than 300 μM, less than 200 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM, less than 1 μM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM.

Alternatively, in certain embodiments, the invention relates to the fusion protein according to the invention, wherein the fusion protein binds to G-CSF-R with a binding affinity ranging from 0.1 nM to 1 mM, from 0.1 nM to 500 μM, ranging from 0.1 nM to 100 μM, ranging from 0.1 nM to 50 μM, ranging from 0.1 nM to 25 μM, ranging from 0.1 nM to 10 μM, ranging from 0.5 nM to 10 μM or ranging from 1 nM to 10 μM.

In another embodiment, the invention relates to the fusion protein according to the invention, wherein the fusion protein has G-CSF-like activity. In another embodiment, the invention relates to the fusion protein according to the invention, wherein the fusion protein has G-CSF-like activity, in particular wherein the G-CSF-like activity comprises at least one, preferably at least two, more preferably at least three, most preferably all of the following activities: (i) induction of granulocytic differentiation of HSPCs; (ii) induction of the formation of myeloid colony-forming units from HSPCs; (iii) induction of the proliferation of NFS-60 cells; and/or (iv) activation of the downstream signaling pathways MAPK/ERK and/or JAK/STAT.

In another embodiment, the invention relates to the fusion protein according to the invention, wherein the fusion protein induces the proliferation of NFS-60 cells.

In another embodiment, the invention relates to the fusion protein according to the invention, wherein the fusion protein induces the proliferation of NFS-60 cells. In another embodiment, the invention relates to the fusion protein according to the invention, wherein the fusion protein induces the proliferation of NFS-60 cells in a culture at a half maximal effective concentration (EC50) of less than 100 μg/mL, preferably less than 50 μg/mL, preferably less than 20 μg/mL, preferably less than 15 μg/mL, preferably less than 10 μg/mL, preferably less than 9 μg/mL, preferably less than 8 μg/mL, preferably less than 7 μg/mL, preferably less than 6 μg/mL, preferably less than 5 μg/mL, preferably less than 4 μg/mL, preferably less than 3 μg/mL, preferably less than 2 μg/mL, preferably less than 1 μg/mL, preferably less than 0.75 μg/mL, preferably less than 0.5 μg/mL, preferably less than 0.25 μg/mL or preferably less than 0.1 μg/mL.

In another embodiment, the invention relates to the fusion protein according to the invention, wherein the fusion protein induces the proliferation and/or differentiation of cells comprising one or more G-CSF receptor on the cell surface.

In another aspect, the invention relates to a polynucleotide encoding the protein or the fusion protein according to the invention. That is, the polynucleotide may encode any protein or fusion protein that falls within the scope of the present invention. Similarly, the invention provides for a polynucleotide comprising a polynucleotide encoding a protein or fusion protein of the invention as described herein.

The term “polynucleotide” as used herein refers to a polymeric form of nucleotides of any length, either ribonucleotides or deoxyribonucleotides. This term refers only to the primary structure of the molecule. Thus, this term includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide.

The term “encoding”, as used herein, like in the terminology “a polynucleotide encoding the protein or fusion protein according to the invention”, refers to the capacity of such polynucleotide to produce a protein or fusion protein upon transcription and translation of the coding sequence contained in such polynucleotide in a target host cell.

Unless otherwise indicated, established methods of recombinant gene technology were used as described, for example, in Sambrook, Russell “Molecular Cloning, A Laboratory Manual”, Cold Spring Harbor Laboratory, N.Y. (2001) which is incorporated herein by reference in its entirety.

In a preferred embodiment, the invention relates to a polynucleotide according to the invention, wherein the polynucleotide is operably linked to at least one promoter capable of directing expression in a cell. Promoters are usually restricted to directing expression of polynucleotides in a certain cell type, organism, or group of organisms. Thus, the at least one promoter may be any promoter that directs expression of the polynucleotide of the invention in a suitable cell.

The term “promoter”, as used herein, refers to a DNA region to which RNA polymerase binds to initiate transcription of a polynucleotide. With respect to the present invention, the promoter may be any promoter that is functional in a respective host cell. Typically, RNA polymerases differ in sequence and structure between organisms or groups of organisms and therefore only initiate transcription at compatible promoters. The promoter may be a constitutive or an inducible promoter. A promoter is said to “direct expression in a cell” if the RNA polymerase of the host cell is compatible with the promoter and capable of initiating transcription. The person skilled in the art is aware of promoters that are compatible with a particular host cell.

The term “operably linked” as used herein, means that a polynucleotide, which can encode a gene product, for example the protein, the fusion protein or a polypeptide chain according to the invention, is linked to a promoter such that the promoter regulates expression of the gene product under appropriate conditions.

In yet another aspect, the invention relates to a vector comprising the polynucleotide according to the invention. The polynucleotide according to the invention may be comprised in any vector that can be maintained and/or replicated in a suitable cell. The vector may only comprise the polynucleotide encoding the protein according to the invention, or may be an expression vector that further comprises one or more promoters operably linked to the polynucleotide.

The term “vector,” as used herein, refers to a recombinant nucleic acid designed to carry a polynucleotide of interest to be introduced into a host cell. This term encompasses many different types of vectors, such as cloning vectors, expression vectors, shuttle vectors, plasmids, phage or virus particles, and the like. A typical expression vector may also include, in addition to a coding sequence of interest, elements that direct the transcription and translation of the coding sequence, such as a promoter, enhancer, terminator, and signal sequence.

In a further aspect, the invention relates to a host cell comprising the polynucleotide of the invention or the vector according to the invention. Preferably a host cell comprising the polynucleotide and expressing the protein encoded thereby is provided. The host cell according to the invention may be any type of cell. Thus, the host cell may be an eukaryotic or a prokaryotic cell and may be a single cell or may be part of a multicellular organization or tissue. The host cell may comprise the polynucleotide according to the invention, with or without a promoter operably linked to the polynucleotide, as a linear polynucleotide in free or modified form. Alternatively, the polynucleotide according to the invention, with or without a promoter operably linked to the polynucleotide, may be integrated into the genome of the host cell. The skilled person is aware of methods to integrate polynucleotides into the genome of various organisms. The cell may further comprise a vector according to the invention. The skilled person is aware of methods to introduce linear polynucleotides or vectors into cells of various organisms. Preferably, the cell according to the invention is compatible with the promoter capable of directing expression and, if necessary, can maintain and/or replicate the vector comprising the polynucleotide according to the invention. The skilled person is aware of combinations of cells, promoters and/or vectors that fulfill these criteria.

In one aspect, the present invention relates to a method for producing a protein or fusion protein according to the present invention. The method preferably comprises the steps of: i) cultivating a host cell according to the present invention; and recovering the protein or fusion protein of the invention from the cell culture and/or cell. In other words, the method may comprise the recombinant expression of the protein of the invention in a host cell according to the present invention that comprises the polynucleotide of the invention operably linked to a promoter (e.g. an inducible promoter). The protein or fusion protein may be expressed and subsequently purified by methods known in the art. Preferred methods for production are described in the appended examples. In some embodiments, the protein or fusion protein of interest may be fused to an affinity tag (e.g. a His-tag) that is used for protein purification. The affinity tag may optionally be removed after purification.

In another aspect, the invention relates to a pharmaceutical composition comprising the protein according to the invention, the fusion protein according to the invention, the polynucleotide according to the invention, the vector according to the invention, and/or the cell according to the invention. Preferably, the pharmaceutical composition also comprises a pharmaceutically acceptable carrier.

That is, the protein according to the invention, the fusion protein according to the invention, the polynucleotide according to the invention, the vector according to the invention or the cell according to the invention, or any combination thereof, may be comprised in a pharmaceutical composition that optionally further comprises at least on pharmaceutically acceptable carrier. The term “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In some cases, the pH of the formulation may be adjusted with pharmaceutically acceptable acids, bases or buffers to enhance the stability of the formulated compound or its delivery form.

In a preferred embodiment, the invention relates to a pharmaceutical composition according to the invention, wherein said pharmaceutical composition is administered in combination with a myelosuppressive agent and/or an immunostimulant.

Various agents have been described to cause myelosuppressive effects in the subjects they are administered to, which can, amongst others, result in anemia and neutropenia. Especially chemotherapeutic agents and antiviral agents frequently cause these side-effects. It has been demonstrated before that myelosuppressive effects of such agents can be prevented, treated and/or alleviated by administering the agent causing the myelosuppressive effect together with G-CSF. Thus, the pharmaceutical composition according to the invention may be administered in combination with any myelosuppressive agent, with the aim to prevent, treat and/or alleviate myelosuppressive effects caused by the myelosuppressive agent. The pharmaceutical composition according to the invention may be administered before the myelosuppressive agent, after the myelosuppressive agent or at the same time as the myelosuppressive agent is administered. In a more preferred embodiment, the invention relates to a pharmaceutical composition according to the invention, wherein said pharmaceutical composition is administered in combination with a myelosuppressive agent and/or an immunostimulant, wherein the myelosuppressive agent is a chemotherapeutic agent and/or an antiviral agent.

The pharmaceutical composition according to the invention may further be administered in combination with an immunostimulant. Immunostimulants may be administered to a subject to boost a subject's immune system or to induce the mobilization of stem cells in said subject. Preferably, the immunostimulant may be an interferon, an interleukin, a colony stimulating factor or any other immunostimulant, such as glatiramer, pegademase bovine, plerixafor or elapegademase. In certain embodiments, the immunostimulant may be G-CSF, preferably human G-CSF, or any derivative thereof. That is, a pharmaceutical composition according to the invention may comprise the protein according to the invention and G-CSF, or a derivative thereof, in any ratio. Without being bound to theory, administering the protein according to the invention together with G-CSF, or a derivative thereof, may result in a strong and fast-acting response to G-CSF, or the derivative thereof, followed by a milder long-term response to the more stable protein according to the invention.

The pharmaceutical composition according to the invention may further comprise more than one myelosuppressive agent or immunostimulant or a combination of myelosuppressive agents and immunostimulants.

The myelosuppressive agent may be any myelosuppressive agent that is known in the art. Preferably, the myelosuppressive agent may be an agent taken from a list consisting of: Peginterferon alfa-2a, Interferon alfa-n3, Peginterferon alfa-2b, Aldesleukin, Gemtuzumab ozogamicin, Interferon alfacon-1, Rituximab, Ibritumomab tiuxetan, Tositumomab, Alemtuzumab, Bevacizumab, L-Phenylalanine, Bortezomib, Cladribine, Carmustine, Amsacrine, Chlorambucil, Raltitrexed, Mitomycin, Bexarotene, Vindesine, Floxuridine, Tioguanine, Vinorelbine, Dexrazoxane, Sorafenib, Streptozocin, Gemcitabine, Teniposide, Epirubicin, Chloramphenicol, Lenalidomide, Altretamine, Zidovudine, Cisplatin, Oxaliplatin, Cyclophosphamide, Fluorouracil, Propylthiouracil, Pentostatin, Methotrexate, Carbamazepine, Vinblastine, Linezolid, Imatinib, Clofarabine, Pemetrexed, Daunorubicin, Irinotecan, Methimazole, Etoposide, Dacarbazine, Temozolomide, Tacrolimus, Sirolimus, Mechlorethamine, Azacitidine, Carboplatin, Dactinomycin, Cytarabine, Doxorubicin, Hydroxyurea, Busulfan, Topotecan, Mercaptopurine, Thalidomide, Melphalan, Fludarabine, Flucytosine, Capecitabine, Procarbazine, Arsenic trioxide, Idarubicin, Ifosfamide, Mitoxantrone, Lomustine, Paclitaxel, Docetaxel, Dasatinib, Decitabine, Nelarabine, Everolimus, Vorinostat, Thiotepa, Ixabepilone, Nilotinib, Belinostat, Trabectedin, Trastuzumab emtansine, Temsirolimus, Bosutinib, Bendamustine, Cabazitaxel, Eribulin, Ruxolitinib, Carfilzomib, Tofacitinib, Ponatinib, Pomalidomide, Obinutuzumab, Tedizolid phosphate, Blinatumomab, Ibrutinib, Palbociclib, Olaparib, Dinutuximab, Colchicine, Penicillamine, Indometacin, Cimetidine, Interferon gamma-1b, omega interferon, Interferon alfa-n1, Peginterferon beta-1a, Cepeginterferon alfa-2B, Interferon beta-1b, Interferon Alfa-2a, Recombinant, Natural alpha interferon and Interferon alfa-2b.

By the term “administered”, as used herein, is intended to include any method of delivering the protein according to the invention, the fusion protein according to the invention or the pharmaceutical composition according to the invention to a subject. The protein according to the invention, the fusion protein according to the invention or the pharmaceutical composition according to the invention may be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents (for example, sugars or sodium chloride). Prolonged absorption of the injectable compositions can be brought about by the use in the compositions of agents delaying absorption (for example, aluminum monostearate and gelatin).

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques that yield a powder of the active ingredient plus any additional desired ingredient from a previously sterile-filtered solution thereof.

The protein according to the invention, the fusion protein according to the invention or the pharmaceutical composition according to the invention would be formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners. The protein according to the invention, the fusion protein according to the invention or the pharmaceutical composition according to the invention need not be, but is optionally formulated with one or more agents currently used to prevent or treat the disorder in question. The effective amount of such other agents depends on the amount of the protein according to the invention present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the protein according to the invention, the fusion protein according to the invention or the pharmaceutical composition according to the invention will depend on the type of disease to be treated, the type of protein, polynucleotide, vector and/or cell, the severity and course of the disease, whether the protein according to the invention, the fusion protein according to the invention or the pharmaceutical composition according to the invention is administered for preventive or therapeutic purposes, previous therapy, the patient's clinical history and response to the protein according to the invention, the fusion protein according to the invention or the pharmaceutical composition according to the invention, and the discretion of the attending physician. The protein according to the invention, the fusion protein according to the invention or the pharmaceutical composition according to the invention is suitably administered to the patient at one time or over a series of treatments, for example, by one or more separate administrations, or by continuous infusion or injection. For repeated administrations over several days or longer, depending on the condition, the treatment would generally be sustained until a desired suppression of disease symptoms occurs.

The frequency of dosing will depend on the pharmacokinetic parameters of the protein according to the invention, the fusion protein according to the invention or the pharmaceutical composition according to the invention and the routes of administration. The optimal pharmaceutical formulation will be determined by one of skill in the art depending on the route of administration and the desired dosage. See, for example, Remington's Pharmaceutical Sciences, supra, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

In another aspect of the invention, an article of manufacture containing materials useful for the prevention, treatment and/or alleviation of symptoms of the disorders or conditions described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a pharmaceutical composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the pharmaceutical composition is a protein according to the invention, a fusion protein according to the invention, a polynucleotide according to the invention, a vector according to the invention or a cell according to the invention. The label or package insert indicates that the composition is used for treating the condition of choice.

Moreover, the article of manufacture may comprise (a) a first container with a pharmaceutical composition contained therein, wherein the composition comprises a protein according to the invention, a fusion protein according to the invention, a polynucleotide according to the invention, a vector according to the invention and/or a cell according to the invention; and (b) a second container with a composition contained therein, wherein the composition comprises a further therapeutic agent. The article of manufacture in this embodiment of the invention may further comprise a package insert indicating that the compositions can be used to treat a particular condition.

The protein according to the invention or the fusion protein according to the invention may be used as a medicament or in the manufacture of a medicament. Thus, in another aspect, the invention relates to a protein, a fusion protein, a polynucleotide, a vector, a cell or a pharmaceutical composition according to the invention for use as a medicament. Alternatively, the invention relates to a protein, a fusion protein, a polynucleotide, a vector, a cell or a pharmaceutical composition according to the invention for use in the manufacture of a medicament.

The protein, the fusion protein, the polynucleotide, the vector, the cell or the pharmaceutical composition according to the invention may be used as a medicament to treat, prevent and/or alleviate any medical condition.

It has been demonstrated by the inventors that the protein or the fusion protein according to the invention directly binds to G-CSF-R with high affinity and elicits similar biological responses as G-CSF when binding to G-CSF-R. Thus, it is plausible that the protein according to the invention, the fusion protein according to the invention or a pharmaceutical composition comprising the protein or fusion protein according to the invention may be used instead of G-CSF for a wide range of therapeutic treatments.

Further, it has been demonstrated by the inventors that the designs Boskar_3 and Boskar_4 have granulopoietic activity in mice (Example 15 and FIG. 26).

As used herein, “treatment” (and grammatical variations thereof such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

The term “prevent,” as used herein, includes prophylactic treatment or treatment that prevents one or more symptoms or conditions of a disease, disorder, or conditions described herein, or may refer to a treatment of a pre-disease state. Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “alleviation” as used herein means all actions that decrease at least the degree of parameters related to conditions being treated, e.g., symptoms.

The term “subject” as used herein denotes any animal, preferably a mammal, and more preferably a human. Examples of subjects include humans, non-human primates, rodents, guinea pigs, rabbits, sheep, pigs, goats, cows, horses, dogs and cats.

The term “medicament”, as used herein, is meant to mean and include any substance (i.e., compound or composition of matter) which, when administered to a subject induces a desired pharmacologic and/or physiologic effect by local and/or systemic action.

The terms “condition” and “medical condition” as used herein, indicate the physical status of the body of a subject (as a whole or of one or more of its parts) that does not conform to a physical status of the subject (as a whole or of one or more of its parts) that is associated with a state of complete physical, mental and possibly social well-being. Conditions herein described include but are not limited to disorders and diseases wherein the term “disorder” indicates a condition of the living subject that is associated to a functional abnormality of the body or of any of its parts, and the term “disease” indicates a condition of the living subject that impairs normal functioning of the body or of any of its parts and is typically manifested by distinguishing signs and symptoms. Exemplary conditions include but are not limited to injuries, disabilities, disorders (including mental and physical disorders), syndromes, infections, deviant behaviors of the subject and atypical variations of structure and functions of the body of an individual or parts thereof.

In another aspect, the invention relates to a protein, a polynucleotide, a vector, a cell or a pharmaceutical composition according to the invention for use in increasing stem cell production.

That is, the protein, the fusion protein, the polynucleotide, the vector, the cell or the pharmaceutical composition according to the invention may be used to induce stem cell production. In a preferred embodiment, the invention relates to a protein, a fusion protein, a polynucleotide, a vector, a cell or a pharmaceutical composition according to the invention for use in inducing hematopoiesis. The term “hematopoiesis” as used herein, refers to the highly orchestrated process of blood cell development and homeostasis. Hematopoiesis starts from multipotent hematopoietic stem cells that differentiate into more specialized cell types through a series of progenitor stages. Thus, a protein, a fusion protein, a polynucleotide, a vector, a cell or a pharmaceutical composition according to the invention is said to “induce hematopoiesis” if it induces the activity, differentiation and/or production of hematopoietic stem cells or any cell deriving thereof.

A “stem cell” as used herein describes a cell that can differentiate into other types of cells that are developmentally restricted to specific lineages, and can also divide in self-renewal to produce more of the same type of stem cells. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body to replenish adult tissues. In a developing embryo, stem cells can differentiate into all the specialized cells—ectoderm, endoderm and mesoderm (see induced pluripotent stem cells)—but also maintain the normal turnover of regenerative organs, such as blood, skin, or intestinal tissues.

A molecule, a cell or a composition is said to “increase stem cell production”, if the molecule induces the division of a stem cell in self-renewal. Thus, the protein, the fusion protein, the polynucleotide, the vector, the cell or the pharmaceutical composition according to the invention may induce the division of any type of human stem cell in self-renewal. Preferably, the protein, the fusion protein, the polynucleotide, the vector, the cell or the pharmaceutical composition according to the invention may induce the division of human hematopoietic stem cells in self-renewal.

The present invention refers mainly, but not exclusively to hematopoietic stem cells. Hematopoietic stem cells (HSCs) are the stem cells that give rise to blood cells. This process is called “hematopoiesis” and occurs in the red bone marrow, in the core of most bones. Hematopoiesis is the process by which all mature blood cells are produced. It must balance enormous production needs (the average person produces more than 500 billion blood cells every day) with the need to precisely regulate the number of each blood cell type in the circulation. In vertebrates, the vast majority of hematopoiesis occurs in the bone marrow and is derived from a limited number of hematopoietic stem cells (HSCs) that are multipotent and capable of extensive self-renewal. HSCs give rise to both the myeloid and lymphoid lineages of blood cells. Myeloid and lymphoid lineages both are involved in dendritic cell formation. Cells from the myeloid lineage include monocytes, macrophages, mast cells, neutrophils, basophils, eosinophils, erythrocytes, and megakaryocytes to platelets. Lymphoid cells include T cells, B cells, and natural killer cells.

Within the myeloid lineage, hematopoietic stem cells have differentiated into common myeloid progenitors, which can then further differentiate into megakaryocytes, erythrocytes, mast cells and myeloblasts. Myeloblasts further differentiate into basophils, neutrophils, eosinophils and monocytes.

The myeloblast is a unipotent stem cell, which will differentiate into one of the effectors of the granulocyte series. The stimulation by G-CSF and other cytokines triggers maturation, differentiation, proliferation and cell survival. It is found in the bone marrow.

The term “progenitor cell” as used herein refers to a cell which is able to differentiate into a certain type of cell and which has limited or no ability to self-renew. A “common myeloid progenitor” is a pluripotent cell that is capable of differentiating into white blood cells, red blood cells and platelets. A “neutrophil progenitor” in the sense of the present invention may be any cell that can differentiate into a neutrophil. A “basophil progenitor” in the sense of the present invention may be any cell that can differentiate into a basophil.

Granulocytes are white blood cells that, amongst others, help the immune system to fight off infections and other diseases. They have a characteristic morphology showing large cytoplasmic granules, which can be stained by basic dyes, and a bi-lobed nucleus. Typically granulocytes have a role both in innate and adaptive immune responses in the fight against viral and parasitic infections. As part of the immune response, granulocytes migrate to the site of infection and release a number of different effector molecules, including histamine, cytokines, chemokines, enzymes and growth factors. As a result granulocytes are an integral part of inflammation and have a significant role in the etiology of allergies.

There are four types of granulocytes: basophils, eosinophils, neutrophils and mast cells. Basophils are the least common type of granulocyte, making only 0.5% of the circulating blood leukocytes. They are involved in a number of functions such as antigen presentation, stimulation and differentiation of CD4+ T cells. Eosinophils make up approximately 1% of circulating leukocytes. Eosinophils play an important and varied role in the immune responses and in the pathogenesis of allergic or autoimmune disease. Neutrophils are the most abundant leukocyte found in human blood and form the vanguard of the body's cellular immune response. Mast cells are a type of granulocyte whose granules are rich in heparin and histamine. Mast cells are important in many immune related activities from allergy to response to pathogens and immune tolerance.

In another embodiment, the invention relates to a method for increasing stem cell production in a subject, the method comprising administrating to said subject a protein, a fusion protein, a polynucleotide, a vector, a cell or a pharmaceutical composition according to the invention. In a preferred embodiment, the invention relates to a method for inducing hematopoiesis in a subject, the method comprising administering to said subject a protein, a fusion protein, a polynucleotide, a vector, a cell or a pharmaceutical composition according to the invention.

G-CSF has been previously demonstrated to stimulate the proliferation of granulocytes. Thus, in a more preferred embodiment, the invention relates to a protein, a fusion protein, a polynucleotide, a vector, a cell or a pharmaceutical composition according to the invention for use in increasing the number of granulocytes in a subject. The protein, the fusion protein, the polynucleotide, the vector, the cell or the pharmaceutical composition according to the invention may be used to increase the number of any type of granulocyte. That is, the protein, the fusion protein, the polynucleotide, the vector, the cell or the pharmaceutical composition according to the invention may be used to increase the number of basophils, eosinophils, neutrophils and/or mast cells. In an even more preferred embodiment, the invention relates to a protein, a fusion protein, a polynucleotide, a vector, a cell or a pharmaceutical composition according to the invention for use in increasing the number of neutrophils and/or eosinophils. Example 5 (FIG. 5) shows that the protein variants of the present invention induce the proliferation of the cell line NFS-60. Thus, it can be plausibly assumed that the protein, the fusion protein, the polynucleotide, the vector, the cell or the pharmaceutical composition according to the invention induces similar physiological responses as G-CSF. In consequence, the protein, the fusion protein, the polynucleotide, the vector, the cell or the pharmaceutical composition according to the invention may be used to treat, prevent and/or alleviate any medical condition related to low stem cell production, impaired hematopoiesis, low granulocyte production and/or low neutrophil and/or eosinophil production.

Within the present invention, the protein, the fusion protein, the polynucleotide, the vector, the cell or the pharmaceutical composition according to the invention is said to “increase the number of granulocytes”, if the number of at least one type of granulocyte is increased in a subject upon administration of the protein, the fusion protein, the polynucleotide, the vector, the cell or the pharmaceutical composition according to the invention to said subject. Alternatively, the protein, the fusion protein, the polynucleotide, the vector, the cell or the pharmaceutical composition according to the invention is said to “increase the number of granulocytes”, if the number of at least one type of granulocyte is increased in a cell culture or any other cell-comprising sample when contacting the cells in the cell culture or sample with the protein, the fusion protein, the polynucleotide, the vector, the cell or the pharmaceutical composition according to the invention.

In another embodiment, the invention relates to a method for increasing the number of granulocytes in a subject, the method comprising administering to said subject a protein, a fusion protein, a polynucleotide, a vector, a cell or a pharmaceutical composition according to the invention.

In a further aspect, the invention relates to a protein, a fusion protein, a polynucleotide, a vector, a cell or a pharmaceutical composition according to the invention for use in accelerating neutrophil recovery following hematopoietic stem cell transplantation.

Low levels of neutrophils in a subject results in a weak immune system and makes said subject more susceptible to, for example, infectious diseases. A molecule, a cell or composition is said to “accelerate neutrophil recovery”, if the molecule induces the production of neutrophils in a subject upon administration of the molecule, cell or composition to said subject. G-CSF is frequently administered to subjects that received hematopoietic stem cell transplantations with the goal to accelerate neutrophil recovery in said subjects. Thus, the protein, the fusion protein, the polynucleotide, the vector, the cell or the pharmaceutical composition according to the invention may be administered to a subject that received hematopoietic stem cell transplantations. The protein, the fusion protein, the polynucleotide, the vector, the cell or the pharmaceutical composition according to the invention may be administered to the subject at any time point after the transplantation.

The term “hematopoietic stem cell transplantation” as used herein, refers to the transplantation of multipotent hematopoietic stem cells, usually derived from bone marrow, peripheral blood, or umbilical cord blood. It may be autologous (the patient's own stem cells are used), allogeneic (the stem cells come from a donor) or syngeneic (from an identical twin). It is most often performed for patients with certain cancers of the blood or bone marrow, such as multiple myeloma or leukemia. In these cases, the recipient's immune system is usually destroyed with radiation or chemotherapy before the transplantation. Infection and graft-versus-host disease are major complications of allogeneic hematopoietic stem cell transplantation. Hematopoietic stem cell transplantation remains a dangerous procedure with many possible complications; it is reserved for patients with life-threatening diseases. As survival following the procedure has increased, its use has expanded beyond cancer to autoimmune diseases and hereditary skeletal dysplasias; notably malignant infantile osteoporosis and mucopolysaccharidosis.

In another embodiment, the invention relates to a method for accelerating neutrophil recovery following hematopoietic stem cell transplantation in a subject, the method comprising administering to said subject a protein, a fusion protein, a polynucleotide, a vector, a cell or a pharmaceutical composition according to the invention.

In yet another aspect, the invention relates to a protein, a fusion protein, a polynucleotide, a vector, a cell or a pharmaceutical composition according to the invention for use in preventing, treating, and/or alleviating myelosuppression resulting from a chemotherapy and/or radiotherapy.

The term “myelosuppression” refers to a reduction in blood-cell production by the bone marrow. It commonly occurs after chemotherapy or radiation therapy. Cytotoxic chemotherapy and/or radiotherapy for the treatment of cancer cause a range of side effects that adversely affect the health and quality of life of a subject. One such side effect is myelosuppression, where chemotherapy and/or radiotherapy may massively deplete bone marrow progenitor cells resulting in anemia, neutropenia, and/or thrombocytopenia. Subjects suffering from myelosuppression may experience complications such as fatigue, dizziness, bruising, hemorrhage, and potentially fatal opportunistic infections. Consequently, drug dosage and/or frequency may be limited to abrogate these complications, but in turn, compromising the effectiveness of the treatment. G-CSF has been administered to subjects receiving chemotherapy and/or radiotherapy to prevent the emergence of chemotherapy-induced and/or radiotherapy-induced myelosuppression, as well as to treat subjects or alleviate the symptoms of subjects that already suffer from chemotherapy-induced and/or radiotherapy-induced myelosuppression. Based on the preserved G-CSF receptor binding site on the protein according to the invention and its demonstrated G-CSF-like activity, it is plausible to assume that the protein, the fusion protein, the polynucleotide, the vector, the cell or the pharmaceutical composition according to the invention may be used in preventing, treating, and/or alleviating the symptoms of myelosuppression resulting from chemotherapy and/or radiotherapy.

The terms “chemotherapy” or “cytotoxic chemotherapy” as used herein, refers to the treatment of cancer using specific chemical agents or drugs that are destructive of malignant cells and tissues. Also, “chemotherapy” refers to the treatment of disease using chemical agents or drugs that are toxic to the causative agent of the disease, such as a virus, bacterium, or other microorganisms.

The terms “radiotherapy” and “radiation therapy” as used herein, refers to a therapy using ionizing radiation, generally as part of cancer treatment to control or kill malignant cells and normally delivered by a linear accelerator. Radiation therapy may be curative in a number of types of cancer if they are localized to one area of the body. It may also be used as part of adjuvant therapy, to prevent tumor recurrence after surgery to remove a primary malignant tumor (for example, early stages of breast cancer). Radiation therapy is synergistic with chemotherapy, and has been used before, during, and after chemotherapy in susceptible cancers.

In another embodiment, the invention relates to a method for preventing, treating, and/or alleviating myelosuppression resulting from a chemotherapy and/or radiotherapy in a subject, the method comprising administering to said subject a protein, a fusion protein, a polynucleotide, a vector, a cell or a pharmaceutical composition according to the invention.

In a further aspect, the invention relates to a protein, a fusion protein, a polynucleotide, a vector, a cell or a pharmaceutical composition according to the invention for use in treating a subject having neutropenia.

Neutropenia is characterized by an abnormally low concentration of neutrophils, a certain type of white blood cells, in the blood of a subject. As a result, subjects suffering from neutropenia have a weakened immune system and are more susceptible to infectious diseases.

The term “neutropenia” as used herein refers to a decrease or small number of neutrophils in the blood compared to normal. For example, the World Health Organization defines neutropenia as a condition of having an absolute neutrophil cell count (ANC) of about 2000 cells/μL or less. Thus, as used herein a subject suffering from neutropenia is one having an ANC of about 2000 cells/μL or less, for example 1000 cells/μL or even less than 500 cells/μL. Neutropenia may be caused by depressed production or increased peripheral destruction of neutrophils. The most common neutropenias are iatrogenic, resulting from the widespread use of cytotoxic or immunosuppressive therapies for cancer treatment or control of autoimmune disorders. Other causes of neutropenia include induction by drugs, hematological diseases including idiopathic, cyclic neutropenia, Chediak-Higashi syndrome, aplastic anemia, infantile genetic disorders, tumor invasion such as myelofibrosis, nutritional deficiency; infections such as tuberculosis, typhoid fever, brucelloisis, tularemia, measles, infectious mononucleosis, malaria, viral hepatitis, leishmaniasis, AIDS, antineutrophil antibodies and/or splenetic or lung trapping, autoimmune disorders, Wegner's granulomatosis, acute endotoxemia, hemodialysis, and cardiopulmonary bypass. The present invention applies to any acquired and inherited neutropenic conditions.

G-CSF has been proven effective in the treatment of neutropenia, as it has been demonstrated to induce the proliferation of neutrophils. Based on the preserved G-CSF receptor binding site on the protein according to the invention and its demonstrated G-CSF-like activity, it is plausible to assume that the protein, the fusion protein, the polynucleotide, the vector, the cell or the pharmaceutical composition according to the invention may also be used in the treatment of neutropenia. As mentioned above, chemotherapy may be a cause for neutropenia. However, the protein, the fusion protein, the polynucleotide, the vector, the cell or the pharmaceutical composition according to the invention may be used to treat neutropenia caused by any other reason.

In another embodiment, the invention relates to a method for treating neutropenia in a subject, the method comprising administering to said subject a protein, a fusion protein, a polynucleotide, a vector, a cell or a pharmaceutical composition according to the invention.

In another aspect, the invention relates to a protein, a fusion protein, a polynucleotide, a vector, a cell or a pharmaceutical composition according to the invention for use in treating neurological disorders.

The receptor G-CSF-R has been shown to be not only present on the surface of hematopoietic stem cells and cells deriving thereof, but also to be present on certain neurons. For example, it has been shown that G-CSF can be used in the treatment of cerebral ischemia to reduce the infarct volume of acute stroke in a rat model [14]. Further, G-CSF may enhance the recovery of humans from a stroke through neuroprotective mechanisms or neurorepair [15]. G-CSF has also been shown to improve spatial learning performance and to markedly reduce amyloid deposition in hippocampus and entorhinal cortex in a murine model of Alzheimer's disease [16]. Thus, the protein, the fusion protein, the polynucleotide, the vector, the cell or the pharmaceutical composition according to the invention may be used in the treatment of neurological disorders, preferably in the treatment on cerebral ischemia and/or Alzheimer's disease.

The term “neurological disorder” as used herein is defined as disease, disorder or condition which directly or indirectly affects the normal functioning or anatomy of a subject's nervous system. Within the present invention, the protein, the polynucleotide, the vector, the cell or the pharmaceutical composition according to the invention may preferably be used in the treatment of cerebral ischemia and/or Alzheimer's disease.

The term “cerebral ischemia” as used herein is defined as insufficient cerebral blood flow resulting in inadequate delivery of oxygen and glucose to the brain. As used herein, it is meant to be synonymous with stroke, which is the clinical syndrome of rapid onset of focal (or global or subarachnoid hemorrhage) cerebral deficit, with no apparent cause other than a vascular one.

“Alzheimer's disease” (AD) is defined as a chronic neurodegenerative disease that usually starts slowly and gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self care, and behavioral issues. As a person's condition declines, they often withdraw from family and society. Gradually, bodily functions are lost, ultimately leading to death. Although the speed of progression can vary, the typical life expectancy following diagnosis is three to nine years.

In another embodiment, the invention relates to a method for treating neurological disorders in a subject, the method comprising administering to said subject a protein, a fusion protein, a polynucleotide, a vector, a cell or a pharmaceutical composition according to the invention.

In another aspect, the invention relates to a protein, a fusion protein, a polynucleotide, a vector, a cell or a pharmaceutical composition according to the invention for use in stem cell mobilization, preferably in mobilization of hematopoietic stem cells (e.g. CD34⁺ stem cells).

Hematopoietic stem cell transplantations have been successfully applied for treating several cancerous and non-cancerous conditions. The vast majority of hematopoietic stem cells is located in the bone marrow, where hematopoiesis takes place. Only small numbers of hematopoietic stem cells are found in peripheral blood. However, the yield of hematopoietic stem cells from peripheral blood can be boosted with daily subcutaneous injections of G-CSF, serving to mobilize stem cells from a donor's bone marrow into the peripheral circulation. As a consequence, hematopoietic stem cells can be extracted from blood in higher numbers, making the direct harvest of hematopoietic stem cells from bone marrow dispensable. Based on the preserved G-CSF receptor binding site on the protein according to the invention and its demonstrated G-CSF-like activity, it is plausible to assume that the protein, the fusion protein, the polynucleotide, the vector, the cell or the pharmaceutical composition according to the invention may be used for the mobilization of stem cells in a donor. In a preferred embodiment, the invention relates to a protein, a fusion protein, a polynucleotide, a vector, a cell or a pharmaceutical composition according to the invention for use in hematopoietic stem cell mobilization. Preferably, the protein according to the invention, a fusion protein according to the invention or the pharmaceutical composition according to the invention may be combined with one or more other stem cell mobilizing agents. Thus, in a preferred embodiment, the invention relates to a protein according to the invention, a fusion protein according to the invention or the pharmaceutical composition according to the invention for use in stem cell mobilization, wherein the protein according to the invention, the fusion protein according to the invention or the pharmaceutical composition according to the invention is administered in combination with at least one additional stem cell mobilizing agent. Non-limiting examples of stem cell mobilizing agents are AMD3100, GRO beta, VLA-4 inhibitor, fucoidan, BI05192, CXCR4 and SDF-1.

The term “stem cell mobilization” as used herein, refers to the recruitment of hematopoietic stem cells (HSCs) from the bone marrow into peripheral blood following treatment with chemotherapy and/or cytokines. The release of HSCs from the bone marrow is a physiological phenomenon for the protection of HSCs from toxic injury, as circulating cells can re-engraft bone marrow, or to maintain a fixed number of HSCs in the bone marrow (homeostatic mechanism). In fact, trafficking to blood is an important death pathway to regulate the steady-state number of HSCs [21]. Bone marrow cells also enter peripheral blood in response to stress signals during injury and inflammation of hematopoietic and non-hematopoietic tissues [22-24].

In another embodiment, the invention relates to a method for mobilizing stem cells in a subject, the method comprising administering to said subject a protein, a fusion protein, a polynucleotide, a vector, a cell or a pharmaceutical composition according to the invention.

In one aspect, the invention also relates to a protein, a fusion protein, a polynucleotide, a vector, a cell or a pharmaceutical composition according to the invention for use in mobilization of CD34⁺ hematopoietic progenitor cells from the bone marrow into the peripheral blood. The invention also provides for a method for mobilizing CD34⁺ hematopoietic progenitor cells in a subject, the method comprising administering to said subject a protein, a fusion protein, a polynucleotide, a vector, a cell or pharmaceutical composition according to the invention.

It is to be understood that for the medical treatments described above, a subject is preferably administered with the protein according to the invention, the fusion protein according to the invention or the pharmaceutical composition according to the invention, wherein the pharmaceutical composition comprises the protein or fusion protein according to the invention. However, a subject in need may also be administered with the polynucleotide according to the invention, the vector according to the invention or the cell according to the invention, for example in gene or cell therapy method as commonly known in the art.

In another aspect, the invention relates to a protein according to the invention or a fusion protein according to the invention as an additive in a cell culture, i.e. the use of a protein according to the invention or a fusion protein according to the invention as a cell culture additive.

That is, the protein according to the invention or the fusion protein according to the invention may be added at any concentration to any type of culture medium that is used for the culturing of any type of cell. The term “cell culture”, as used herein, refers to an in vitro population of viable cells under cell cultivation conditions, i.e. under conditions wherein the cells are suspended in a culture medium that will allow their survival and preferably their growth. The cells in the cell culture may be any cell type. Preferably, the cell in the cell culture is a cell comprising human G-CSF-R on the cell surface. More preferably, the cell in the cell culture is a human cell, even more preferably a human hematopoietic stem cell or any cell deriving thereof or a human neural stem cell or any cell deriving thereof.

The terms “medium” and “culture medium”, as used herein, refer to a solution containing nutrients that nourish cells. Typically, these solutions provide essential and nonessential amino acids, vitamins, energy sources, lipids, and trace elements required by the cell for minimal growth and/or survival. The solution may also contain components that enhance growth and/or survival above the minimal rate, including hormones and growth factors. The solution is preferably formulated to a pH and salt concentration optimal for cell survival and proliferation. Within the present invention, the protein according to the invention may be added to any culture medium. Preferably, the protein according to the invention is added to a culture medium that is suitable for culturing mammalian cells, such as culture media that are based on DMEM, RPMI 1640, MEM, IMDM, Alpha MEM, StemPro-34 and/or DMEM/F-12.

The term “additive” as used herein refers to a molecule that is added to a cell culture, preferably to the culture medium.

In a preferred embodiment, the invention relates to the use of a protein according to the invention or a fusion protein according to the invention for stimulating the proliferation and/or differentiation of cells in a cell culture.

That is, the protein according to the invention or the fusion protein according to the invention may be used to stimulate the proliferation and/or differentiation of any kind of cell. Example 5 (FIG. 5) shows that the protein variants of the present invention induce the proliferation of the cell line NFS-60. Further, Example 7 (FIG. 10) shows that the protein variants of the invention can induce the differentiation of HSPCs into myeloid CFUs. Thus, in a more preferred embodiment, the invention relates to a protein according to the invention or a fusion protein according to the invention, wherein the protein stimulates the proliferation and/or differentiation of cells in a cell culture, wherein the cells in the cell culture comprise the G-CSF receptor on the cell surface, even more preferably, wherein the cells in the cell culture are hematopoietic stem cells or any cell deriving thereof, even more preferably, wherein the cells in the cell culture are common myeloid progenitors or any cell deriving thereof, and most preferably, wherein the cells in the cell culture are myeloblasts or any cell deriving thereof.

The term “proliferation” as used herein in reference to cells can refer to a group of cells that can increase in number over a period of time. The term “differentiation” as used herein, refers to the cellular development of a cell from a less specialized stage towards a more mature specialized cell. The less specialized cell may be a stem cell or a progenitor cell. Within the present invention, the less specialized cell may be a hematopoietic stem cell or any cell deriving thereof that is not terminally differentiated or a neural stem cell or any cell deriving thereof that is not terminally differentiated. The protein according to the invention or the fusion protein according to the invention is said to “stimulate proliferation and/or differentiation” of a cell, if the protein or the fusion protein according to the invention increases the rate with which a cell, or population of cells, proliferates and/or differentiates.

In another aspect, the invention relates to a method for proliferating and/or differentiating cells in a cell culture by contacting said cells with the protein according to the invention or the fusion protein according to the invention.

That is, the protein according to the invention or the fusion protein according to the invention may be used in a method for proliferating and/or differentiating any type of cell in a cell culture. The cells in the cell culture may be contacted with the protein according to the invention or the fusion protein according to the invention by any means. For example, the cells in the cell culture may be contacted with the protein or the fusion protein according to the invention by adding the protein or fusion protein according to the invention to the culture medium. The protein or fusion protein according to the invention may be added to the culture medium at any concentration. Preferably, the protein or fusion protein according to the invention may be used to proliferate and/or differentiate cells comprising the G-CSF receptor on the cell surface. Thus, in a preferred embodiment, the invention relates to a method for proliferating and/or differentiating cells in a cell culture by contacting said cells with the protein or fusion protein according to the invention, wherein the cells comprise the G-CSF receptor on the cell surface. In a more preferred embodiment, the invention relates to a method for proliferating and/or differentiating cells in a cell culture by contacting said cells with the protein or fusion protein according to the invention, wherein the cells in the cell culture are hematopoietic stem cells or any cell deriving thereof. In an even more preferred embodiment, the invention relates to a method for proliferating and/or differentiating cells in a cell culture by contacting said cells with the protein or fusion protein according to the invention, wherein the cells in the cell culture are common myeloid progenitors or any cell deriving thereof. In a most preferred embodiment, the invention relates to a method for proliferating and/or differentiating cells in a cell culture by contacting said cells with the protein or fusion protein according to the invention, wherein the cells in the cell culture are myeloblasts or any cell deriving thereof.

The term “contacting,” as used herein, refers to the act of bringing two or more components together in direct contact by dissolving, mixing, suspending, blending, slurrying, or stirring. Within the present invention, the protein or fusion protein according to the invention is contacted with a cell, if the protein according to the invention and the cell are in such close proximity that the protein according to the invention may bind to a receptor on the cell surface, preferably to G-CSF-R.

### BRIEF DESCRIPTION OF THE FIGURES

FIG. 1. Topological manipulation strategies to simplify the G-CSF fold. (A) Topological rearrangement strategy via de novo design of short loops to replace the long, disordered loops. The structure on the left shows the human G-CSF fold (PDB: 5GW9), while the model on the right shows the simplified design topology Boskar_4 (SEQ ID NO:5). (B) Scaffold hopping strategy by retrofitting the receptor binding site (black patch represents binding site II) onto diverse scaffolds with locally geometrically matched backbones. Top pane shows G-CSF bound to its receptor (PDB: 2D9Q). Bottom pane shows two diverse geometrically compatible scaffolds with simpler topologies; Sohair (SEQ ID NO:14) and Moevan (SEQ ID NO:6), on the left and right sides, respectively.

FIG. 2. The most active designs are all more stable than wild type G-CSF. Top to bottom panes show melting curves and circular dichroism spectra, and are ordered as Moevan (SEQ ID NO:6), Disohair_2 (SEQ ID NO:19; C₂ symmetric dimer), Boskar_4 (SEQ ID NO:5) and GCSF, respectively.

FIG. 3. Disohair_2 (SEQ ID NO:19) is substantially more resistant to neutrophil elastase digestion than G-CSF and Moevan (SEQ ID NO:6). SDS-PAGE analysis of digestion products after neutrophil elastase incubation for 5, 15 and 30 minutes. While, Moevan (left) and G-CSF (right) get completely digested after 5 minutes of incubation, Disohair_2 (middle) is more resistant to proteolysis by neutrophil elastase.

FIG. 4. Boskar_4 (SEQ ID NO:5) is substantially more resistant to neutrophil elastase digestion than G-CSF. SDS-PAGE analysis of digestion products after neutrophil elastase incubation for 5, 15 and 30 minutes.

FIG. 5. Concentration-dependent cell proliferation curves of NFS-60 cells in presence of five different newly designed proteins and rhGCSF (recombinantly expressed in E. coli). Each data point represents the average of three independent measurements with the standard deviation indicated by error bars. The curves were analyzed using a four-parameter sigmoid fit.

FIG. 6. Concentration-dependent cell proliferation curves of NFS-60 cells to evaluate the functional stability of rhGCSF and the most active design Boskar_4 (SEQ ID NO:5), following 4-week incubation at 4° C. Each data point represents the average of three independent measurements with the standard deviation indicated by error bars. The curves were analyzed using a four-parameter sigmoid fit.

FIG. 7. Expression of the protein designs in E. coli. The designs DiSohair_1 (SEQ ID NO:18), DiSohair_2 (SEQ ID NO:19) and Moevan (SEQ ID NO:6) and the recombinant G-CSF variant filgrastim were expressed in E. coli, respectively. Total lysates of the cells (left) and the soluble protein fraction (middle) were separated by SDS-PAGE. On the right, the separation of total lysates of uninduced cells is shown.

FIG. 8. Evaluation of the biological activity of the Boskar_3 (SEQ ID NO:4) and Boskar_4 (SEQ ID NO:5) in human hematopoietic stem and progenitor cells. Representative FACS profiles (A) and neutrophil surface marker expression of treated CD34+ HSPCs as assessed by FACS (B) after 14 days of culture. Data represent mean±standard deviation performed in triplicates from two different healthy donor samples. (C) Representative cytospin slides images of cells generated using liquid culture myeloid differentiation for 14 days.

FIG. 9. Evaluation of the biological activity of the DiSohair_2 (SEQ ID NO:19) and Movean (SEQ ID NO:6) in human hematopoietic stem and progenitor cells (HSPCs). Representative FACS profiles (A) and neutrophil surface marker expression of treated CD34+ HSPCs as assessed by FACS (B) after 14 days of culture. Data represent mean±standard deviation performed in triplicates from two different healthy donor samples. (C) Representative cytospin slides images of cells generated using liquid culture myeloid differentiation for 14 days.

FIG. 10. Generation of colony forming units (CFU) from HSPCs stimulated with designed proteins. (A) Quantification of CFU numbers and (B) representative images of colonies induced by rhG-CSF, or designs n CD34+ HSPCs after 14 days in culture. Data represent mean±standard deviation of triplicates from two independent experiments.

FIG. 11. Evaluation of the ability of designs to phosphorylate signaling proteins that are normally activated downstream of G-CSFR upon G-CSFR activation. Intracellular levels of phospho-ERK1/2 (p44/42 MAPK), phospho-STAT3 and phospho-STAT5 in CD34+ HSPCs treated with rhG-CSF or designs. Data derived from two independent experiments.

FIG. 12. NMR solution structure agrees with design models of Moevan and Sohair. A) Ribbon representation of the NMR structure ensemble is overlaid on cartoon design model of Moevan. B) Ribbon representation of the NMR ensemble is overlaid on cartoon design model of Sohair.

FIG. 13. Chromatographic specific elution peak for Boskar_4. Affinity purified Boskar_4 from supernatant of a 2.5-Litre E. coli expression culture (straight line represent baseline drift).

FIG. 14. Chromatographic specific elution peak for rhG-CSF. Affinity purified refolded rhG-CSF from the denatured insoluble fraction of a 2.5-Litre E. coli expression culture (straight line represent baseline drift).

FIG. 15. Chromatographic specific elution peak for Moevan. Affinity purified Moevan from supernatant of a 2.5-Litre E. coli expression culture (straight line represent baseline drift).

FIG. 16. Chromatographic specific elution peak for diSohair2. Affinity purified DiSohair_2 from supernatant of a 2.5-Litre E. coli expression culture (straight line represent baseline drift).

FIG. 17. The design model shows atomic-level agreement with its NMR solution structure. (A) Boskar4 solution structure shows an ensemble deviation from the average structure of 1.34 Å, and 2.59 Å from the designed coordinates. The design model is shown against the NMR ensemble and the box plot shows the deviations across the ensemble. (B) The backbone atoms RMSD of the binding epitope averaged at 0.80 Å, while all-atom RMSD of averaged at 1.52 Å, highlighting the design precision. The design model residues are shown against the NMR ensemble, and the box plot shows the deviations across the ensemble.

FIG. 18. Analytical size-exclusion elution profile of Boskar3 shows almost equipartition between monomeric and dimeric species. Calibration curve shown in grey.

FIG. 19. Supplementary FIG. 6. Analytical size-exclusion elution profile of Boskar4 shows dimeric (minor) and monoric (major) species. Calibration curve shown in grey.

FIG. 20. The designs directly bind the human G-CSF receptor. SPR sensograms of rhG-CSFR binding kinetics by (A) rhG-CSF, (B) diSohair2, (C) diSohair_control, (D) Moevan, (E) Moevan_t2, and (F) Moevan_control. Moevan_control and diSohair_control showed no measurable binding (C, F). Curves represent binding model fits.

FIG. 21. Analytical size-exclusion elution profile of Moevan shows a monomeric (major) and dimeric (minor) species. Calibration curve shown in grey. (Bottom) Analytical size-exclusion elution profile of diSohair2 shows a dimeric and tetrameric species. Calibration curve shown in grey.

FIG. 22. G-CSFR-deficient primary stem cells (G-CSFR KO), show abolished proliferative responses to either rhG-CSF or the designs. Experiment was performed twice in triplicates.

FIG. 23. Intracellular levels of phospho-AKT (Thr308), phospho-ERK1/2 (p44/42 MAPK), phospho-STAT3 (Tyr705), and phospho-STAT5 (Tyr694) in CD34+ HSPCs treated with rhG-CSF or the designs (see Materials and Methods). Geometric mean of the expression intensity of each phospho-protein (GeoMean intensity) is shown on the y-axis. The experiment was performed twice.

FIG. 24. Reactive oxygen species (ROS) assay of granulocytes generated on day 14 of liquid culture. Data show mean±standard deviation.

FIG. 25. Phagocytosis kinetic analysis using IncuCyte ZOOM System of granulocytes generated on day 14 of liquid culture. Lines represent mean, shades represent ±standard deviation. Solid and dashed lines represent activated neutrophils with or without pHrodo green E. coli bioparticles conjugate, respectively.

FIG. 26. C57BL/6 mice were treated with PBS, rhG-CSF, Boskar3, or Boskar4 (n=7 per group for each condition). Mice treated with Boskar3 or Boskar4 show significant increase in Gr-1+ and CD11b+ cells in the bone marrow compared to PBS-treated mice. Data show mean±standard deviation. (*, p<0.05 vs. the PBS group).

### EXAMPLES

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

### Example 1: In Silico Design of Protein Variants

The first stage of the inventors approach was to convert the two bundle-spanning loops between α-helices A and B and α-helices C and D into two short de novo designed loops, which obligates the redesign of an up-up-down-down four-helix-bundle into an up-down-up-down four-helix-bundle. This is expected to bring the contact order of an idealized bundle to a theoretical minimum, and also decreases the domain sequence length by almost a third of the wild-type sequence length. This was followed by three more stages of redesign to improve the core packing, optimization of the loop landing sites to the best scoring new loop compositions, and redesign all of the newly surface exposed residues after removing the loops. This was done while maintaining site II conformationally and compositionally fixed.

Geometric Search Algorithm

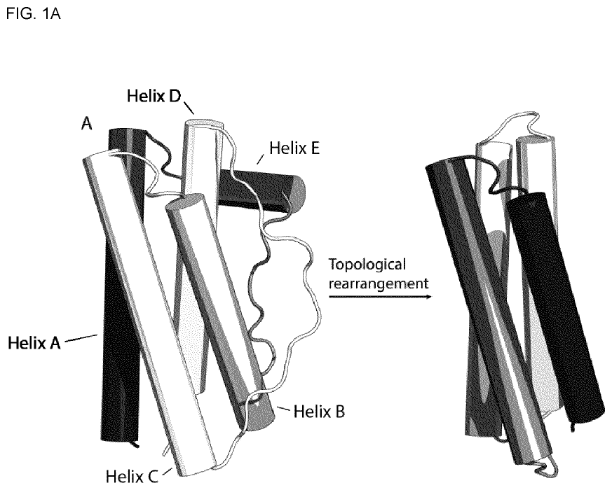

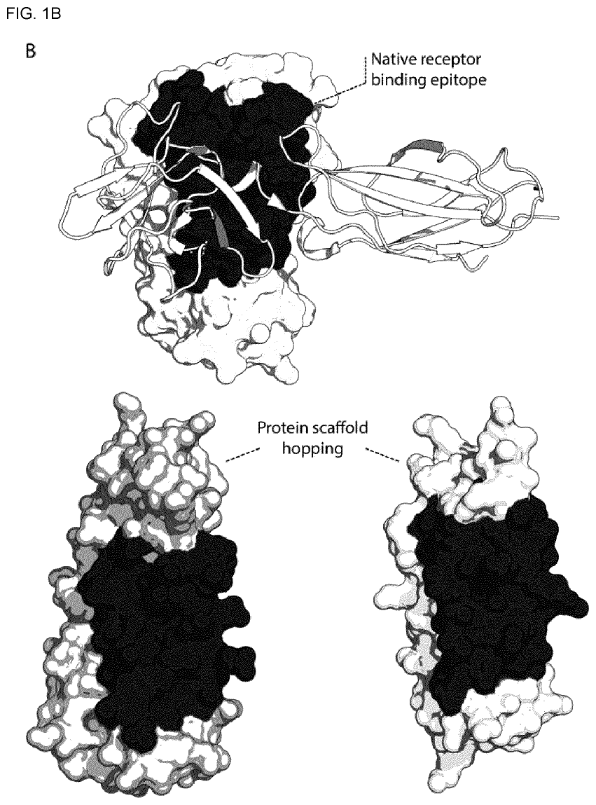

At the first stage the inventors aimed at systematically searching the PDB for finding accommodating structural scaffolds to host the essential site II residues, namely: K16, E19, Q20, R22, K23, D27, D109, and D112 (FIG. 1A). The aim was to match backbone dihedrals and 3D backbone positions of the query residues to similar substructures in the PDB. To simplify the search space, the residues were assumed to lie on two discontinuous segments in the subject structures (i.e.: segment 1: 16-27, segment 2: 109-112). This has allowed us to extend a previous loop-grafting routine, originally developed finding loops across discontinuous secondary structure [37], to generically search for pairs of structural segments disconnected by any number of intervening residues. The extended routine aims at finding minimizing arguments for three objective functions. The routine scans across every protein in a protein structure database searching for disjoint fragments, that have minimal internal orientation difference to the query fragments, minimal internal spacing difference to the query fragment, and minimal average backbone dihedrals deviation from the query fragments. These three functions were applied in a tiered search scheme, to systematically scan the PDB for candidate domains to host the disembodied residues. The top hits were re-ranked by their aligned RMSD to the query substructures, and smallest and topologically simplest hits where chosen for the design stage.

Loop Design

Novel loops were constructed through the automatic modeling of three- or four-residue long loops was performed covering the all sequence combinations of the involved residue types, which comprised: G, D, P, S, L, N, T, E, K for three-residue-loops, and G, D, P, S, L, N, T, K for four-residue-loops. A novel loop energetics evaluation routine was devised to perform adaptively directed generalized-ensemble sampling, based on a theoretical framework demonstrating the approximate equivalence of serial tempering to systematic umbrella sampling. The conformational homogeneity, quantified through a measure of local mean square structural deviations, of the resulting simulation trajectories was used to rank the candidate loop sequences for stability.

Sequence Optimization for Stability Enhancement

Sequence and conformer sampling were performed to the designs upon retrofitting the selected scaffolds with disembodied residues, using the RosettaScripts framework. In addition to an RMSD constraint on the binding epitope, a previously described core packing protocol was used. That comprised steps of interleaved Monte Carlo sequence and side chain and backbone conformer sampling iterations. The sequence sampling was directed to most core residues and to solvent-exposed hydrophobic residues. The scoring functions used were the talaris2013 energy function and the packstat packing score. While the energy function was used to bias the sampling towards lower energy decoys, the top decoys were forwarded for further evaluation based on the packing quality, where the latter was further judged by the ruggedness of the radial distribution function g(r) as given by the definite integral ∫₀⁴|dg(r)/dr|dr.

In Silico Affinity Maturation

Mutations were systematically sampling for residues around the binding epitope of the artificial GCSF to lower the potential energy of the modelled receptor-design complexes. The modeled complexes were based on the native GCSF-GCSFR complex (PDB:2D9Q), where the design models were aligned by their binding pharmacophore to the native ligand and further annealed in implicit solvent to refine their docked posses. For a more accurate evaluation for the binding free energy of the complexes, potential of mean force (PMF) [37] simulations were used to estimate the binding free energy (to the of the GCSF receptor CRH domains) generated decoys.

As a result, eight protein designs, namely Boskar_1 (SEQ ID NO:2), Boskar_2 (SEQ ID NO:3), Boskar_3 (SEQ ID NO:4), Boskar_4 (SEQ ID NO:5), Moevan (SEQ ID NO:6), Sohair (SEQ ID NO:14), Disohair_1 (SEQ ID NO:18) and Disohair_2 (SEQ ID NO:19) have been obtained with the strategy described above.

### Example 2: Expression and Purification

The synthetic genes encoding the protein variants designed in Example 1 were ordered and cloned in-frame with an N-terminal hexa-His-tag and a thrombin cleavage site into the NdeI and XhoI sites of the pET28a(+) expression vector harboring a kanamycin resistance gene as a selection marker. The plasmids were transformed by heat-shock in chemically competent E. coli BL21(DE3) cells. For protein expression, the cells were grown in LB medium and expression was induced with IPTG at OD₆₀₀ of 0.5-1 followed by incubation overnight at 25° C. For expression of isotopically labeled protein, a pre-culture in LB medium was grown, cells were collected, washed twice in PBS buffer, and resuspended in M9 minimal medium (240 mM Na₂HPO₄, 110 mM KH₂PO₄, 43 mM NaCl), supplemented with 10 μM FeSO₄, 0.4 μM H₃BO₃, 10 nM CuSO₄, 10 nM ZnSO₄, 80 nM MnCl₂, 30 nM CoCl₂, and 38 μM kanamycin sulfate to an OD₆₀₀ of 0.5-1. After 40 minutes of incubation at 25° C., 2.0 gram ¹⁵N-labelled ammonium chloride (Sigma-Aldrich cat. nr. 299251) and 6.25 gram ¹³C D-glucose (Cambridge Isotope Laboratories, Inc. cat. nr. CLM-1396) were added to a 2.5 L culture. Following another 40 minutes of incubation, IPTG was added to 1 mM final concentration to induce overnight expression. Cells were collected by centrifugation at 5,000 g for 15 minutes, lysed using a Branson Sonifier S-250 (Fisher Scientific) in hypotonic 50 mM Tris-HCl buffer supplemented with cOmplete protease cocktail (Sigma-Aldrich cat. nr. 4693159001) and 3 mg of lyophilized DNase I (5200 U/mg; Applichem cat. nr. A3778). The insoluble fraction was pelleted by centrifugation at 25,000 g for 50 minutes, and the soluble fraction was filtered (0.45 μm filter pore size) and directly applied to a Ni-NTA column. For wild-type G-CSF, from the expressed protein was extracted from the insoluble fraction of lysed E. coli cells by stirring the pellet in 8 M guanidinium chloride solution for 2 hours at 4° C. The mixture was gradually diluted to 1 M guanidinium chloride in 4 steps over 4 hours, and loaded directly onto a Ni-NTA column. A 5 mL HisTrapFF immobilized nickel column (GE Healthcare Life Sciences cat. nr. 17-5255-01) was used for this purpose, washed consecutively with 30 mL 150 mM NaCl, 30 mM Tris buffer (pH 8.5) containing 0, 30 and 60 mM imidazole. Bound protein was eluted with a linear gradient from 60-500 mM imidazole and fractions were collected. The eluate was concentrated using 3 kDa MWCO centrifugal filters (Merck Millipore cat. nr. UFC901024) and loaded onto a Superdex 75 gel filtration column (GE Healthcare Life Sciences cat. nr. 17517401) equilibrated with gel filtration buffer, which was always Phosphate Buffered Saline (PBS) pH 7.4 which is favorable for NMR, CD, and cell culture. An ÄktaFPLC system (GE Healthcare Life Sciences) was used for all chromatography runs.

The inventors expressed all newly designed proteins in E. coli, where all protein variants were efficiently expressed as soluble protein. After the IMAC and preparative size exclusion chromatography, the non-optimized final purification yield of the designs was at least 15 mg per liter culture.

In comparison, filgrastim (recombinant human G-CSF) is only insolubly expressed in E. coli and has to be refolded from inclusion bodies prior to purification. The optimized production yield in the pharmacopoeia-mandated expression host, E. coli, was 3.2 mg/Liter culture [11].

### Example 3: Biophysical Analyses

Thermal unfolding was measured by CD spectroscopy monitoring loss of secondary structure. The temperature was monitored and regulated by a Peltier element which was connected to the CD spectroscopy unit. The temperature was measured in the cuvette jacket that is made of copper. Samples (0.5 mL) of concentrations between 0.3 and 6 mg/mL were loaded into 2 mm path length cuvettes. Spectral scans of mean residual ellipticity were done at a resolution of 0.1 nm, across the range of 240-195 nm. Melting curves tracked the mean residual ellipticity at a wavelength of 222 nm across a temperature range of 20 to 100° C. Melting temperature was extracted as the value of Tₘ (where

$$\frac{1}{2} = \frac{{T}_{max} - {T}_{m}}{{T}_{max} - {T}_{min}}$$

), where an inflection is observed.

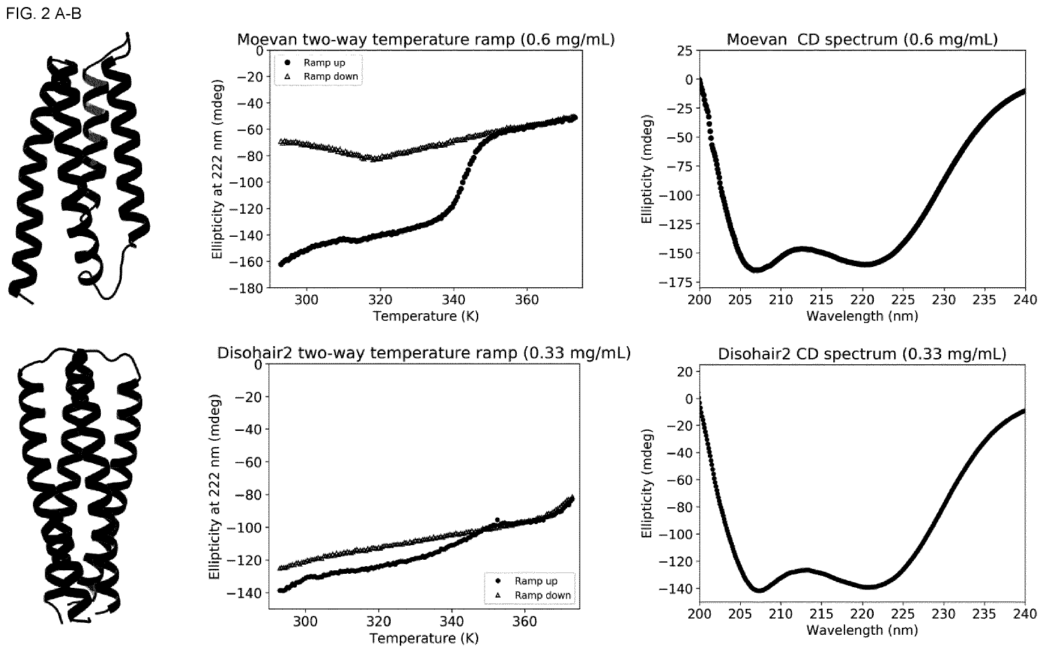

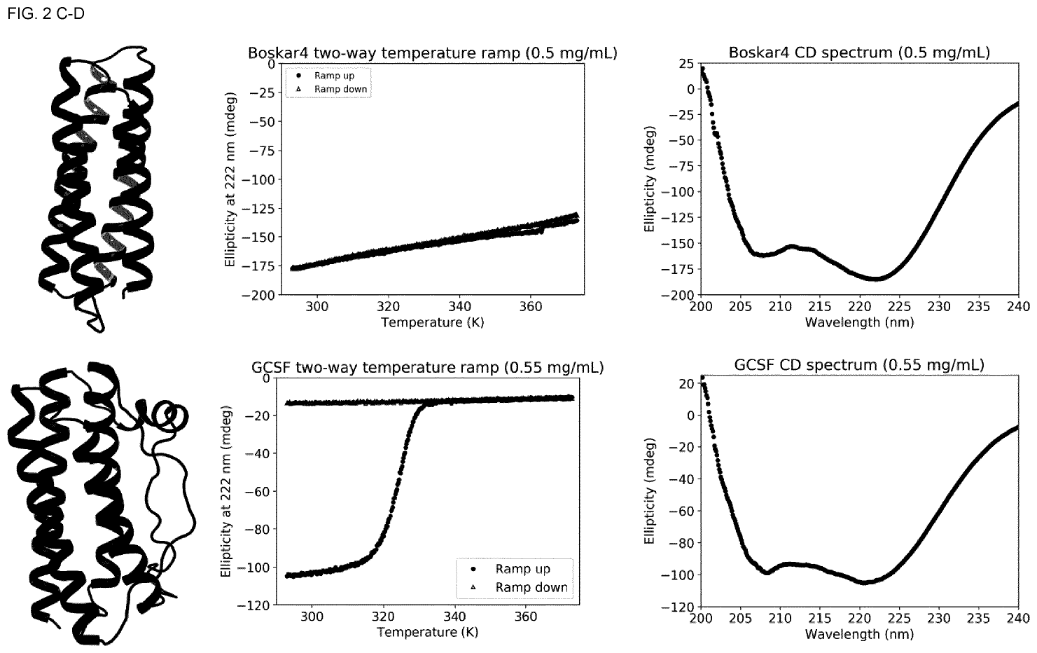

Circular dichroism spectra of diSohair_2 (SEQ ID NO:19), Moevan (SEQ ID NO:6) and Boskar_4 (SEQ ID NO:5) showed strong alpha-helical content, with characteristic minima of almost double intensities compared to that of G-CSF at the same concentration. Strong NMR signal dispersion also indicated well folded proteins for Moevan (SEQ ID NO:6), Sohair (SEQ ID NO:14), and Boskar_4 (SEQ ID NO:5). Thermal melting measured by circular dichroism of the most active design Boskar_4 (SEQ ID NO:5), and diSohair_2 (SEQ ID NO:19), showed thermal stability up to 100° C. accompanied by only a slight decrease in helicity, which was fully reversible upon cooling (FIGS. 2 B and C). The melting curves of wild-type G-CSF however showed complete thermal unfolding of the protein with a mid-transition at 57° C. Unfolding of wild-type G-CSF was irreversible as the unfolded protein aggregated and formed precipitates (FIG. 2D).

### Example 4: Protease Sensitivity Assay

Previous studies have established the negative feedback loop of granulopoiesis, where GCSF-induced neutrophils in-turn release neutrophil elastase (NE) that strongly antagonises GCSF through its GCSF-directed protease activity. NE concentration in serum was shown to be directly correlated to neutrophil count, and is demonstrated to be the major degrading protease of GCSF [18, 19]. The inventors have thus compared three of their protein designs against filgrastim USP standard to assess their NE degradation sensitivity.

Purified human neutrophil elastase was obtained from Enzo Life Science (cat. nr.: BML-SE284-0100). The elastase was reconstituted in PBS buffer (pH 7.4) to a stock concentration of 20 IU/mL. Digestion reactions were conducted in PBS buffer with final concentrations of 300 μg/mL of the protein of interest and 1 U/mL of neutrophil elastase. The reaction mixture was incubated at 37° C. and digestion samples were withdrawn, immediately mixed with SDS sample buffer (450 mM Tris HCl, 12% Glycerol, and 10% SDS) and flash-frozen in liquid nitrogen bath to stop the reaction, after 5, 15 and 30 minutes from the reaction start. Frozen samples were then heated at 85° C. for 10 minutes before loading on Novex™ 16% Tricine Protein Gels (ThermoFisher Scientific; cat. nr. EC6695BOX). The SDS-PAGE gels were incubated overnight in fixing solution (30% ethanol, 10% acetic acid), and then stained using colloidal coomassie dye.

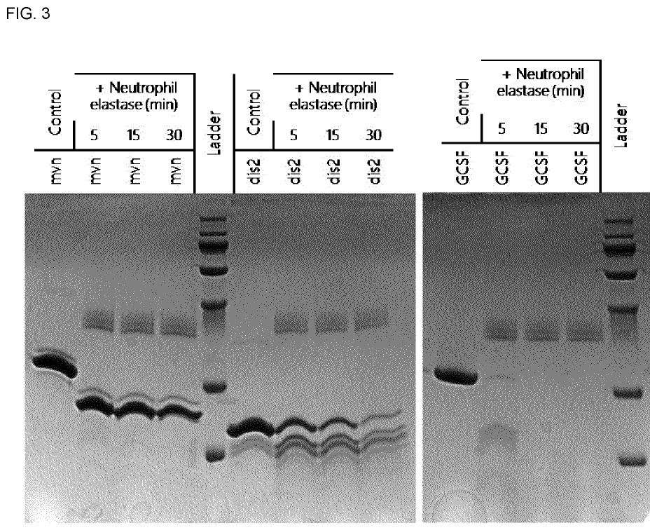

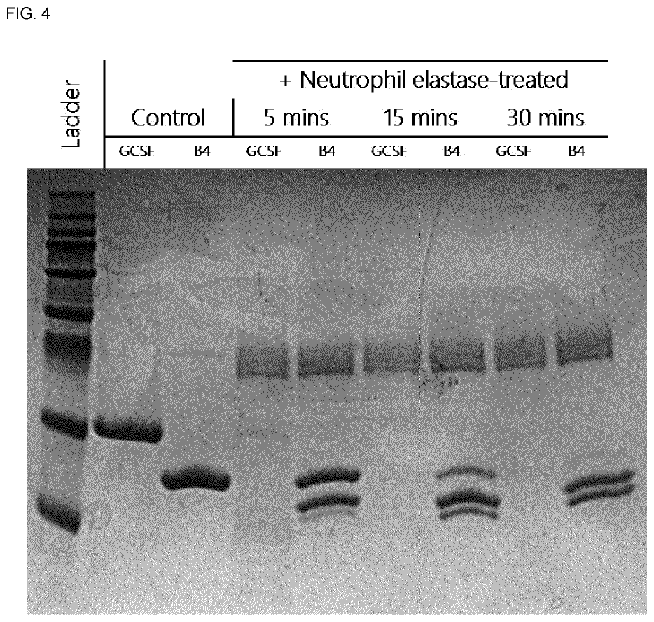

The results show that Moevan (SEQ ID NO:6) and human G-CSF are very susceptible to NE proteolysis, while Boskar_4 (SEQ ID NO:5) and Disohair_2 (SEQ ID NO:19) are much more resistant to NE (FIGS. 3 and 4).

### Example 5: In-Cell Activity Testing

For testing the functionality of the newly designed protein variants in cells, the inventors analyzed the proliferation of NFS-60 cells. The growth and maintenance of viability of this murine myeloblastic cell line is dependent on IL-3. NFS-60 cells are also highly responsive to IL-3, GM-CSF, G-CSF, and erythropoietin and therefore commonly used to assay human and murine G-CSF activity.

NFS-60 cells were cultured in GM-CSF-containing RPMI 1640 medium ready-to-use, supplemented with L-glutamine, 10% KMG-5 and 10% FBS (cls, cell line services). Before each assay, cells were pelleted and washed three times with cold non-supplemented RPMI 1640 medium. After the last washing step, cells were diluted at a density of 6×10⁵ cells/mL in RPMI 1640 containing glutamine and 10% FBS. In order to analyze cell proliferation, NFS-60 cells were grown in the presence of varying concentrations of G-CSF wild-type and designed variants. For this, fivefold dilution series were prepared from stock solutions of wild type G-CSF (40 ng/mL) and newly designed protein variants (40 μg/mL) in RPMI 1640 medium supplemented with glutamine and 10% FBS. 75 μL of each dilution were mixed with the same volume of washed cells in a 96 well plate yielding a final cell density of 3×10⁵ cells/mL and G-CSF concentrations varying from 0.00001-20 ng/mL for wild type and 0.01-20,000 ng/μL for the designs. Each 96 well plate contained triplicates of each dilution and the according blanks, including wells containing cells seeded in RPMI 1640 medium supplemented with L-glutamine, 10% KMG-5 and 10% FBS (cls, cell line services) and wells containing medium solely. Following incubation for 48 h at 37° C. and 5% CO₂, 30 μL of the redox dye resazurin (CellTiter-Blue® Cell Viability Assay, Promega) was added to the wells and incubation was continued for another hour. Cell viability was measured by monitoring the fluorescence of each well at a H4 Synergy Plate Reader (BioTek) using the following settings: excitation=560/9.0, Emission=590/9.0, read speed=normal, delay=100 msec, measurements/data Point=10. The data were analyzed and curves were plotted applying a four-parameter sigmoid fit using SigmaPlot (Systat Software).

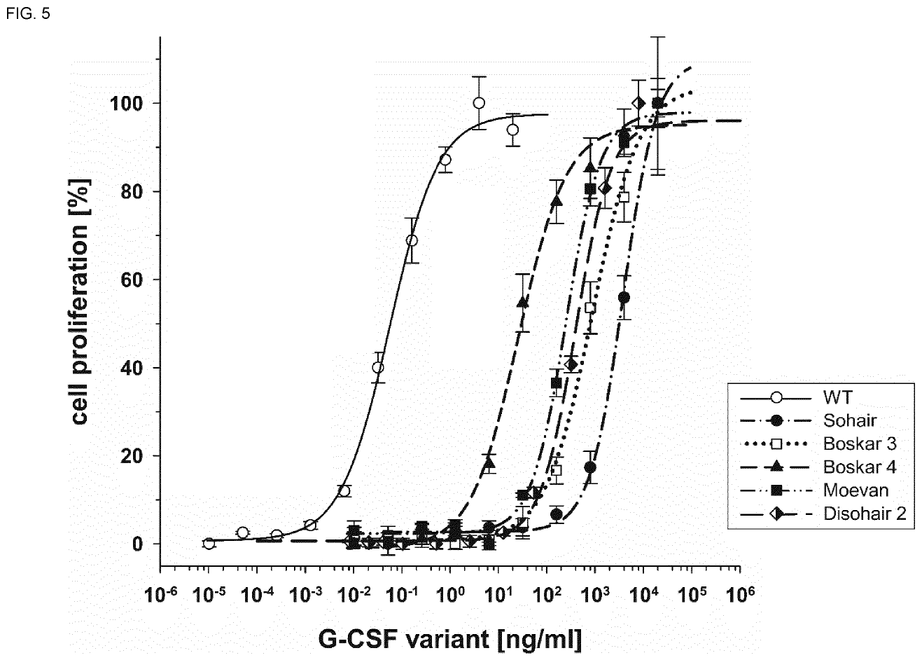

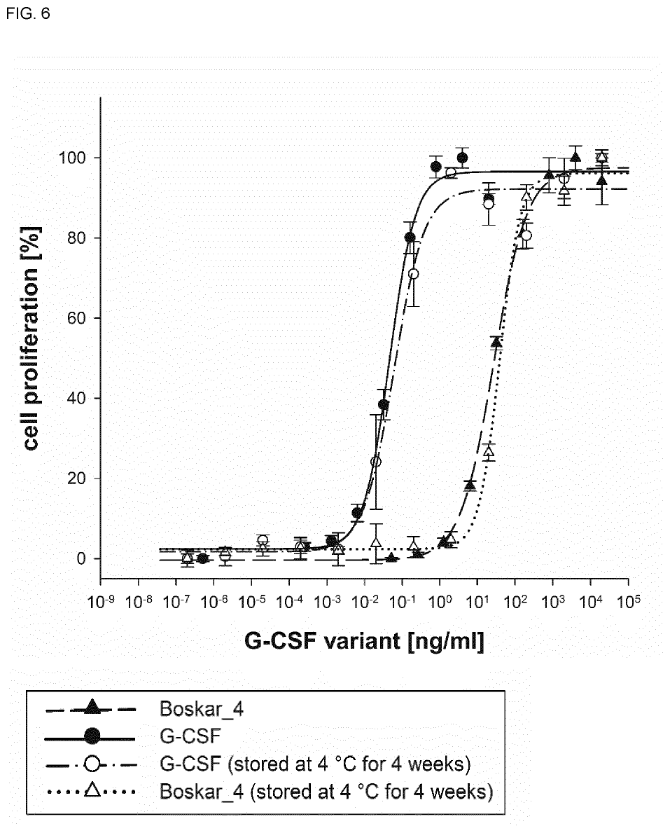

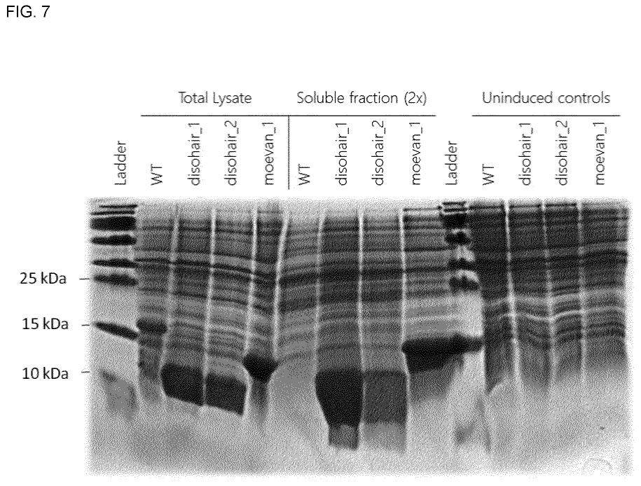

Five different designs (Boskar_3 (SEQ ID NO:4), Boskar_4 (SEQ ID NO:5), Sohair (SEQ ID NO:14), Moevan (SEQ ID NO:6) and Disohair_2 (SEQ ID NO:19)) were analyzed in comparison to wild-type human G-CSF. In the assay, variant Boskar_4 (SEQ ID NO:5) had the highest activity of the five designs followed by Moevan (SEQ ID NO:6), Disohair_2 (SEQ ID NO:19), Boskar_3 (SEQ ID NO:4) and Sohair (SEQ ID NO:14) (FIG. 5). All protein variants, as well as human G-CSF, retained their stability over a storage period of 4 weeks at 4° C. (FIG. 6), although wild type G-CSF started to aggregate at a concentration of 1 mg/ml.

### Example 6: Induction of In Vitro Granulocytic Differentiation of HSPCs

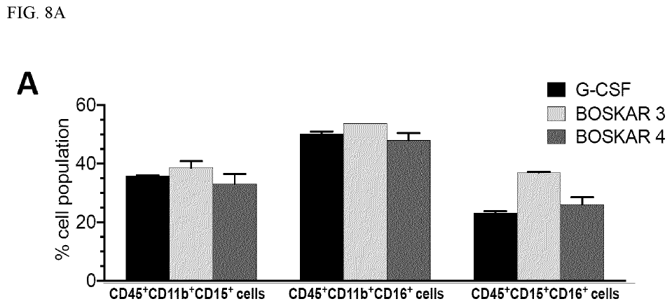

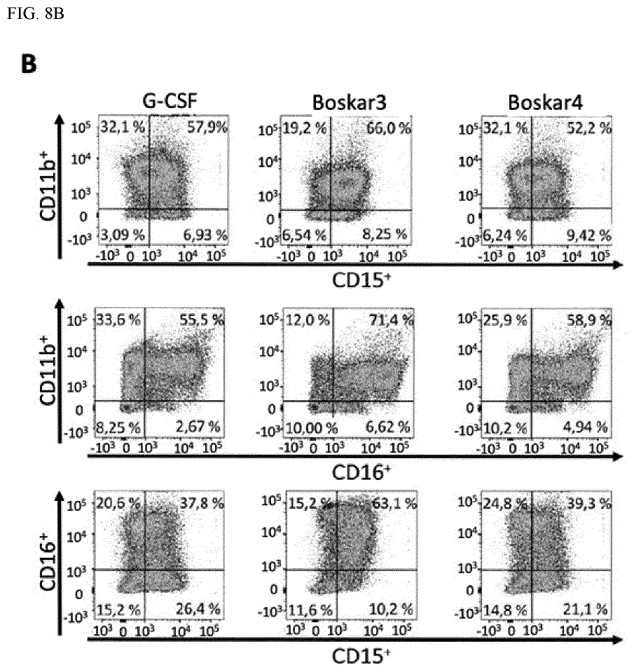

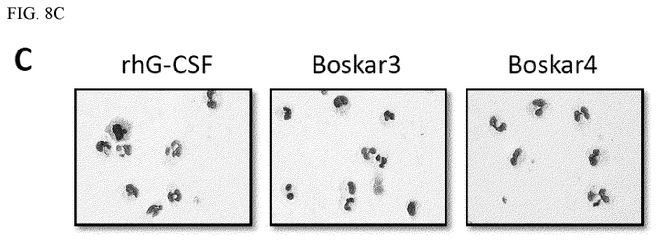

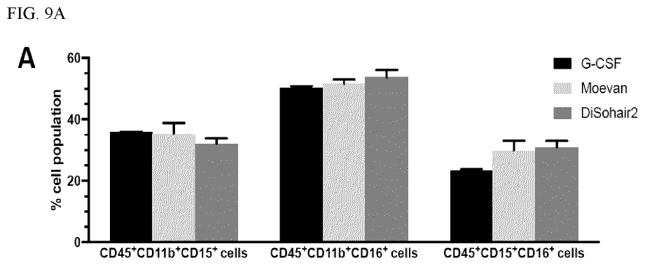

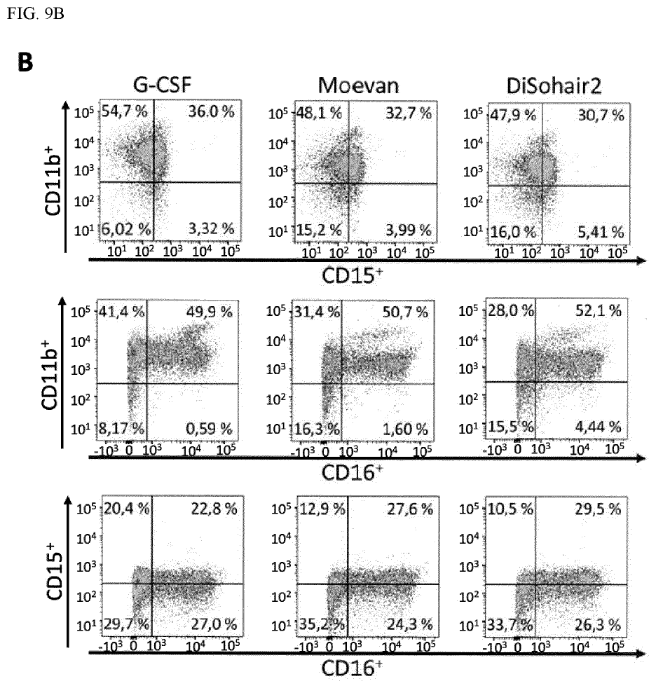

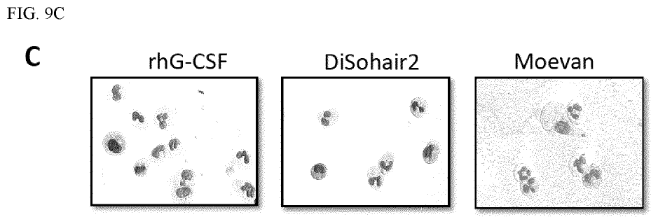

It was first evaluated whether G-CSF-like designs are capable to induce myeloid differentiation of human CD34+ hematopoietic stem and progenitor cells (HSPCs) in vitro. To study in vitro myelopoietic capacity of the designs, human CD34+ HSPCs were isolated from the bone marrow mononuclear cell fraction of two healthy donors by magnetic bead separation using the Human CD34 Progenitor Cell Isolation Kit (Miltenyi Biotech #130-046-703, Germany). CD34+ cells were cultured at a density of 2×10⁵ cells/mL in Stemline II Hematopoietic Stem Cell Expansion medium (Sigma Aldrich, #50192) supplemented with 10% FBS, 1% penicillin/streptomycin, 1% L-glutamine and 20 ng/mL IL-3, 20 ng/mL IL-6, 20 ng/mL TPO, 50 ng/mL SCF and 50 ng/mL FLT-3L. For liquid culture granulocytic differentiation, expanded CD34+ cells (2×10⁵ cells/mL) were incubated for 7 days in RPMI 1640 GlutaMAX supplemented with 10% FBS, 1% penicillin/streptomycin, 5 ng/mL SCF, 5 ng/mL IL-3, 5 ng/mL GM-CSF and 10 ng/mL of rhG-CSF, or 10 μg/mL of each design, respectively. Medium was exchanged every second day. On day 7, medium was changed to RPMI 1640 GlutaMax supplemented with 10% FBS, 1% penicillin/streptomycin and 10 ng/mL rhG-CSF, or 10 μg/mL of each design, respectively. Medium was exchanged every second day until day 14. On day 14, cells were analyzed by flow cytometry using the following antibodies: mouse anti-human CD45 (Biolegend, #304036), mouse anti-human CD11b (BD, #557754), mouse anti-human CD15 (BD, #555402), and mouse anti-human CD16 (BD, #561248) on a FACSCanto II instrument. Of note, FACS analysis revealed differentiation of HSPCs isolated from two healthy donors in myeloid/granulocytic cells, co-expressing cell surface markers of granulocytes, such as CD15+CD11b+, CD16+CD11b+, CD15+CD16+ cells, in the presence of designs to the levels comparable to that of rhG-CSF. HSPCs of healthy donor 1 were stimulated with rhG-CSF, Boskar_3 (SEQ ID NO:4), or Boskar_4 (SEQ ID NO:5) (FIG. 8A, B), HSPCs of healthy donor 2 were treated with rhG-CSF, DiSohair_2 (SEQ ID NO:19), or Moevan (SEQ ID NO:6) (FIG. 9A, B).

It was also analyzed whether myeloid cells generated in the presence of the designs will have the typical cell morphology of mature neutrophils. Cell morphology was evaluated on cytospin preparations. For this, cells were isolated on day 14 of culture, 10×10⁴ cells per cytospin slide were centrifuged at 400 g for 5 min at room temperature using a Thermo Scientific Cytospin 4 Cytocentrifuge. Wright-Giemsa-stained cytospin slides were prepared using Hema-Tek slide stainer (Ames) and evaluated using a Nikon Inverted Microscope. As expected, a vast majority of cells cultured in the presence of rhG-CSF or designs revealed the typical and highly specific morphology of neutrophilic granulocytes with multilobed nuclei (FIG. 8C, 9C).

These data clearly demonstrate biological activity of designs towards granulocytic differentiation of human hematopoietic stem and progenitor cells.

### Example 7: Induction of Formation of Myeloid Colony-Forming Units (CFUs) from HSPCs

It was further tested whether the designs induce the formation of myeloid colony-forming units (CFUs) from healthy donor HSPCs. This would be an additional proof of the biological activity of designs on the hematopoietic stem cells. For this, CD34+ HSPCs at a concentration of 10.000 cells/mL medium were plated in 35 mm cell culture dishes in 1 mL Methocult H4230 medium (Stemcell Technologies) supplemented with 2% FBS, 10 μg/mL of 100× Antibiotic-Antimycotic Solution (Sigma) and 50 ng/mL of rhG-CSF, or 1 μg/mL of Boskar_3, Boskar_4, DiSohair_2 or Moevan, respectively. Cells were cultured at 37° C., 5% CO2. Colonies were counted on day 14.

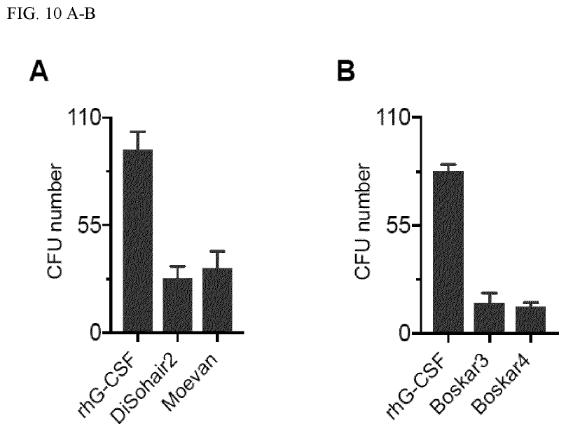

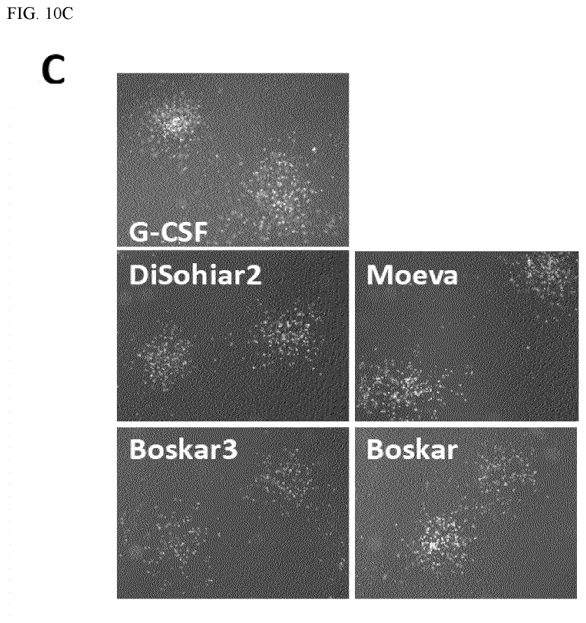

Indeed, myeloid CFUs were observed in the HSPC cultures in the presence of the designed proteins. Although the number of CFU colonies induced by Boskar_3 (SEQ ID NO:4), Boskar_4 (SEQ ID NO:5), Moevan (SEQ ID NO:6) and DiSohair_2 (SEQ ID NO:19) was much lower than the number stimulated by rhG-CSF, the typical myeloid cell morphology of CFUs was visible in all groups (FIG. 10). These data further support granulopoietic activity of design proteins.

### Example 8: Activation of G-CSF Receptor Downstream Intracellular Signaling Pathways in Human Hematopoietic Stem Cells

Binding of rhG-CSF to G-CSFR activates a cascade of intracellular signaling pathways, including phosphorylation of downstream proteins, such as STAT3, STAT5, or MAPK, which ultimately induces granulocytic differentiation of HSPCs. Therefore, it was investigated whether the designs are capable of inducing phosphorylation of these proteins in CD34+ HSPCs. For this, CD34+ cells were cultured in Stemline® II Hematopoietic Stemcell Expansion Medium (Sigma-Aldrich; #50192) supplemented with 10% FBS (Sigma-Aldrich; #F7524; batch-no. BCBW7154), 1% L-Glutamine (Biochrom; #K0283), 1% Pen/Strep (Biochrom; #A2213) and a premixed Cytokine Cocktail containing rh-IL3 (PeproTech; #200-03), rh-IL6 (Novus Biologicals; #NBP2-34901), rh-TPO, rh-SCF (both R&D Systems; TPO #288-TP200; SCF #255-SC-200) and rh-Flt-3L (BioLegend; #550606). The final concentration of IL-3, IL-6 and TPO was 20 ng/ml, and for SCF and Flt-3L 50 ng/ml. On day 6 of culture, serum- and cytokine-starved (3 h) CD34+ HSPCs were treated with rhG-CSF, Moevan (SEQ ID NO:6) or DiSohair_2 (SEQ ID NO:19) (10 μg/mL for Moevan and Di-Sohair_2), respectively, for 10, 15 or 30 min, fixed in 4% PFA (Merck; #P6148) for 15 min at room temperature, and permeabilized for 30 min by slowly adding ice-cold methanol (C. Roth; #7342.1) to a final concentration of 90%. Cells were left overnight in methanol at −20° C. and stained on the next day with specific antibodies recognizing phosphorylated signaling effectors (phospho-Stat3 (Tyr705) (D3A7) XP rabbit mAb (Cell Signaling; #9145); phospho-Stat5 (Tyr694) (C1105) rabbit mAb (Cell Signaling; #9359), and phospho-p44/42 MAPK (Erk1/2) (Thr202/Tyr204) (E10) mouse mAb (Cell Signaling; #9106) or respective isotype control antibody (anti-mouse IgG (H+L), F(ab′)2 fragment (Alexa Fluor® 488 Conjugate) (Cell Signaling; #4408; goat anti-rabbit IgG H+L (Alexa Fluor® 488) (abcam; #ab150077) by incubation for 20 minutes on ice in PBS/2% BSA. After that, cells were washed twice in ice-cold PBS/2% BSA and analyzed by FACS. To determine the background-corrected fluorescent signal from the corresponding phosphorylated proteins, the fluorescent signal of the appropriate isotype control estimated at each time point of stimulation was subtracted from the specific phospho-protein signal.

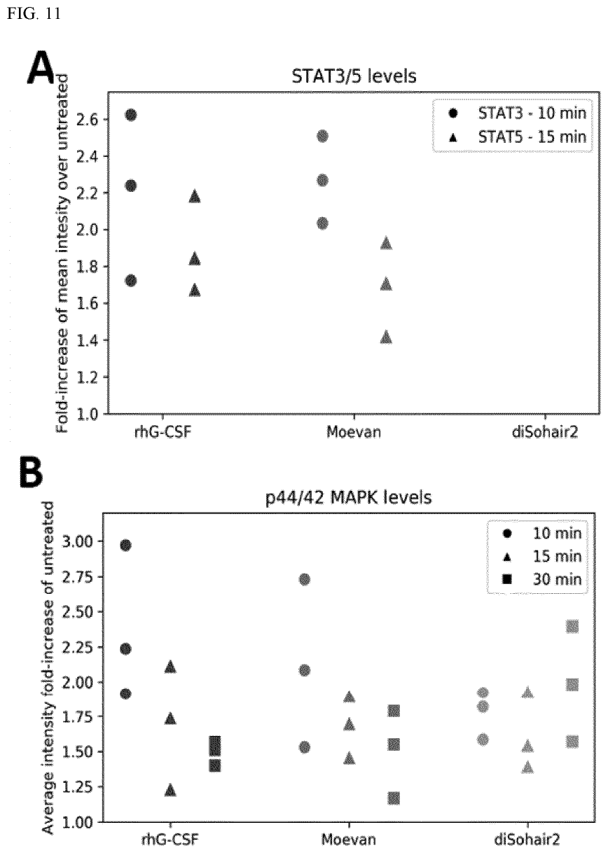

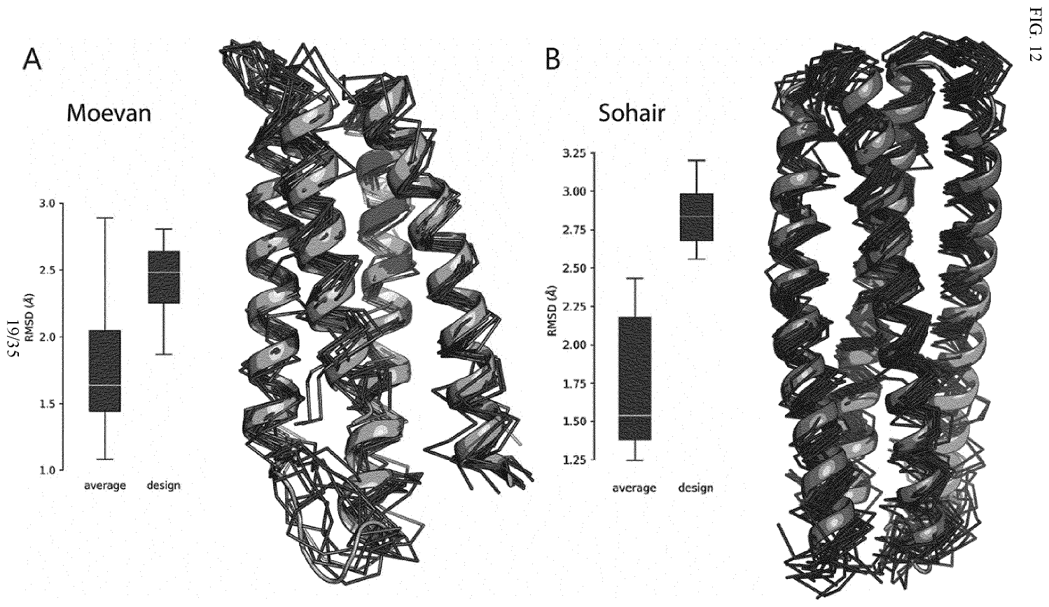

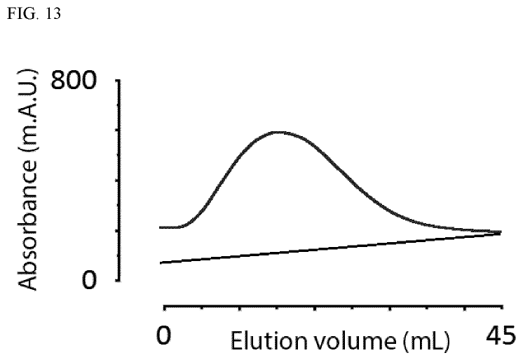

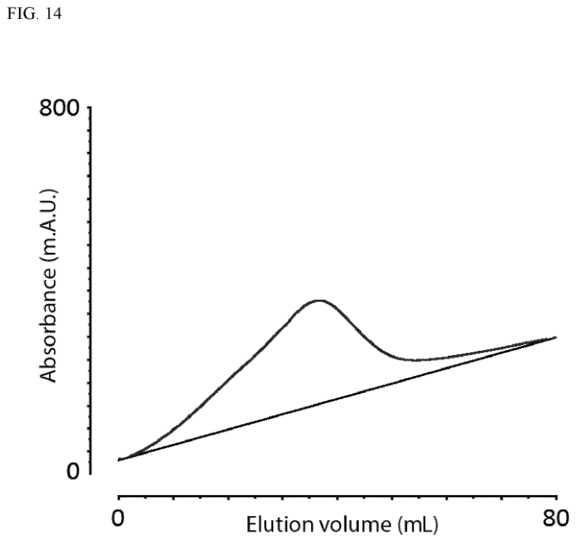

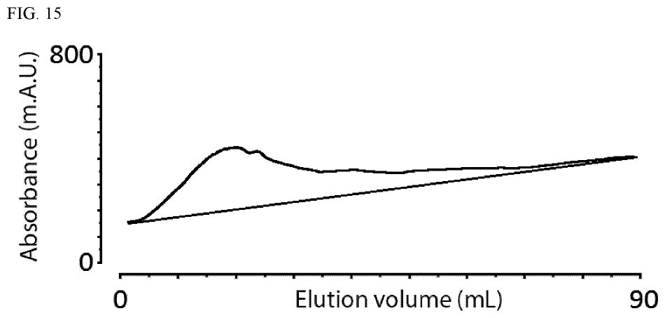

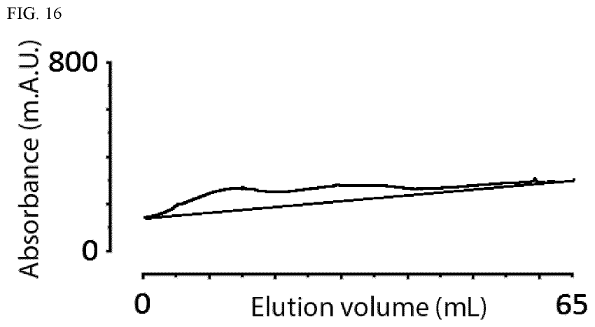

Indeed, time-dependent tyrosine phosphorylation of p44/42 MAPK (Erk1/2) in HSPCs treated with Moevan (SEQ ID NO:6) or DiSohair_2 (SEQ ID NO:19), respectively, was observed to a similar degree as in rhG-CSF treated cells (FIG. 11B). At the same time, Moevan (SEQ ID NO:6) activates tyrosine phosphorylation of STAT3 and STAT5 proteins after 10 and 15 min of treatment, respectively (FIG. 11A). Although the kinetic modes and the degree of activation were different between rhG-CSF and G-CSF mimics, these data strongly demonstrate that G-CSF mimics are capable to activate downstream G-CSF receptor signaling pathways in CD34+ cells. These data suggest that design proteins act through G-CSFR activation upon stimulation of human hematopoietic stem cells.

### Example 9: In-Cell Activity Testing with Further Designs

NFS-60 cells were cultured in IL-3-containing RPMI 1640 medium, supplemented with L-glutamine, 10% KMG-5 and 10% FBS (CLS, cell line services). Before each assay, cells were pelleted and washed three times with cold non-supplemented RPMI 1640 medium. After the last washing step, cells were diluted at a density of 6×10⁵ cells/ml in RPMI 1640 containing glutamine and 10% FBS. In order to analyze cell proliferation, NFS-60 cells were grown in the presence of varying concentrations of G-CSF wild-type and designed variants. For this, five-fold dilution series were prepared from stock solutions of the designs (Moevan t2=60.2 μg/ml, Boskar4 t2=2 μg/ml, bika1=26.8 μg/ml, bika2=1.07 μg/ml, Sohair2_15 rl=26.8 μg/mL, Boskar4_15 rl=26.8 μg/ml, Boskar4_st2=26 μg/mL, Moevan_st2=26 μg/mL) in RPMI 1640 medium supplemented with glutamine and 10% FBS. 75 μl of each dilution were mixed with the same volume of washed cells in a 96-well plate yielding a final cell density of 3×10⁵ cells/ml and designed protein concentrations varying from 0.0001-60,000 ng/mL. Each 96-well plate contained triplicates of each dilution and the according blanks, including wells containing cells seeded in RPMI 1640 medium supplemented with L-glutamine, 10% KMG-5 and 10% FBS (cls, cell line services) and wells containing medium only. For endpoint analysis, following incubation for 48 h at 37° C. and 5% CO₂, 30 μl of the redox dye resazurin (CellTiter-Blue® Cell Viability Assay, Promega) was added to the wells, and incubation was continued for another hour. Cell viability was measured by monitoring the fluorescence of each well at a H4 Synergy Plate Reader (BioTek) using the following settings: excitation=560 nm±9 nm, Emission=590 nm±9 nm, read speed=normal, delay=100 ms, measurements per data point=10. The data were analysed and curves were plotted applying a four-parameter sigmoid fit using SigmaPlot (Systat Software).

The inventors surprisingly found that dimerization of protein designs results in more active variants. For example, it has been demonstrated that the variant boskar4_t2, comprising two boskar_4 variants connected via a 24 amino acid GS-rich linker, induced the proliferation of NFS-60 cells with an EC₅₀ of 4.2 ng/mL. More importantly, the dimeric variant boskar_4_st2, comprising a 6 amino acid GS-linker induced the proliferation of NFS-60 cells even with an EC50 of 0.202 ng/mL (Table 7). In comparison, the parent variant boskar_4 induced the proliferation of NFS-60 cells with an EC₅₀ of 27 ng/mL (Table 5). Variant boskar4_15 rl, comprising a 15 amino linker between helices 2 and 3 induced the proliferation of NFS-60 cells with an EC₅₀ of 48.5 ng/mL (Table 7).

Similarly to boskar_4, dimerization of Moevan also resulted in higher activity. The designs moevan_t2 (24 amino acid GS-linker) and moevan_st2 (6 amino acid GS-linker) induced proliferation of NFS-60 cells with EC₅₀ values of 47.1 ng/mL and 8.89 ng/mL, respectively (Table 5). The parent variant Moevan induced proliferation of NFS-60 cells with an EC₅₀ of 356 ng/mL (Table 7).

Variant disohair2_15 rl comprises two disohair2 designs connected via a 15 amino acid GS-linker. The activity of this variant was increased compared to the variant Disohair_2 (228 ng/mL compared to 396 ng/mL, Tables 5 and 7).

The two designs bika1 and bika2 have been demonstrated to induce the proliferation of NFS-60 cells with an EC₅₀ of 63 ng/mL and 98 ng/mL respectively (Table 7).

### Example 10: Analysis of the Binding Epitope in Boskar_4

To evaluate the structural precision of the design process, the inventors determined the structure of Boskar4. The structure was determined using the CoMAND method (Conformational Mapping by Analytical NOESY Decomposition), a protocol that provides unbiased structure determination driven by a residue-wise R-factor tracking the match between experimental and back-calculated NOESY spectra. In the CoMAND protocol, a 3D-CNH-NOESY spectrum is divided into 1D sub-spectra, each representing contacts to a single backbone amide proton, thus representing the structural environment at and around the respective residue. Spectral decomposition is then performed, which yields the local backbone dihedral angles for all residues where strips are available. In a subsequent stage, the R-factor is used as a selection criterion for frame-picking from equilibrium MD trajectories, yielding the final structure ensemble.

The CNH-NOESY spectra of Boskar4 provided 98 strips, after excluding strips containing overlapped intensities. CoMAND factorization calculations were performed on these strips, yielding backbone dihedrals, that were both consistent with the values predicted from chemical shift profiles by TALOS-N, as well as the lowest energy Rosetta ab initio folding decoy. Refinement was done by running 1 μs of explicit solvent NPT sampling followed by the frame picking step, where the global average R-factor minimization converged after the picking of 12 frames. This final ensemble yielded an average R-factor of 0.36±0.11 over 89 spectra (Table 8). The ensemble deviated by an average of 1.34 Å from the average structure, and 2.59 Å from the design model (FIG. 17A). Locally aligning the NMR ensemble to the designed binding epitope residues showed a backbone RMSD of 0.80 Å and an all-atom RMSD of 1.52 Å (FIG. 17B), thus demonstrating atomic precision in resculpting the binding epitope.

For Moevan, the CNH-NOESY spectra provided sub-spectra 205 for 102 amide protons, with those missing mainly due to unassigned resonances spanning two ranges (residues 1-8 and 65-67) where the latter stretch was a disordered loop in the template structure. The inventors applied CoMAND factorization calculations to these sub-spectra, yielding backbone dihedrals both consistent with the values predicted from chemical shift profiles by TALOS-N and having the lowest energy Rosetta ab initio folding decoy. Due to its high conformational heterogeneity, the refinement simulations for Moevan were carried out under a set of unambiguous distance restraints. During the frame-picking stage, R-factor minimization converged at 17 frames, three of which were rejected on the basis of distance restraint violations, leaving 14 frames constituting the final ensemble. The ensemble deviated by an average of 1.8 Å from the average structure, and 2.5 Å from the design model (FIG. 12A). Locally aligning the NMR ensemble to the G-CSF binding epitope stretches (residues 12-28 and 104-116) resulted in an RMSD of 1.0 Å.

For Sohair, the inventors extracted 146 CNH-NOESY sub-spectra out of a total length of 154 residues (excluding the purification tag). Due to the significant pseudo-symmetry in the sequence and chemical environment, 29 of these had overlapped intensities. Performing CoMAND factorization on the non-overlapped strips, the inventors obtained backbone dihedrals consistent with TALOS-N predictions, which are in turn in line with the dihedral values of the lowest energy Rosetta ab initio folding decoy. The final, refined ensemble compiled by R-factor minimization yielded 19 frames, with an RMSD of 1.8 Å from the average structure. Although the final ensemble has an average RMSD of 2.9 Å to the design model (FIG. 12B), local alignment of the grafted epitope to G-CSF yields a considerably lower average RMSD of 1.5 Å.

Methods:

All spectra were recorded at 310 K on Bruker AVIII-600 and AVIII-800 spectrometers. Backbone sequential and aliphatic side chain assignments were completed using standard triple resonance experiments, while aromatic assignments were made by linking aromatic spin systems to the respective CβH2 protons in a 2D-NOESY spectrum. Structures were calculated using the CoMAND method, which exploits the high accuracy that can be obtained in back-calculating NOESY spectra with indirect 13504 C dimensions. The CoMAND method involves spectral decomposition of one-dimensional sub-spectra extracted from a 3D-CNH-NOESY spectrum. These sub-spectra are chosen from a search area centered on assigned 15506 N-HSQC positions and thus contain only cross-peaks to a specific amide proton. Residues with overlapping search areas were examined separately. In most cases strips with acceptable separation of signals could be obtained. Where this was not possible, the residues were flagged as overlapped and a joint strip constructed by summing those at the estimated maxima of the respective components. These 1D strips were decomposed against a library of spectra back-calculated by systematic sampling over a local dihedral angle space, yielding estimates of backbone and side chain dihedral angles for each residue. In this work however, the inventors have excluded heavily overlapped strips since there were only few overlaps. Later stages of the protocol involve conformer selection aimed at minimizing a quantitative R-factor expressing the match between the experimental strips and back-calculated spectra, or a fold-factor designed to isolate the contribution to the R-factor from long-range NOESY contacts.

For initial model building, unrestrained Rosetta ab initio folding simulations were performed and generated 10,222 decoys. The corresponding CNH-NOESY spectra of these decoys were back-calculated to evaluate the structure-averaged fold-factors. The decoy with the lowest fold-factor was used to seed five independent unrestrained molecular dynamics simulations. These refinement simulations were carried out using the CHARMM36 force field in explicit solvent using the polarizable TIP3P water model. Trajectories of a total length of approximately 1 μs were run, with frames collected every 100 ps. An initial refined ensemble was compiled through a global greedy minimization of the R-factor as previously described, which converged on a total of 12 frames.

### Example 11: Binding of the Protein Designs to G-CSF-R

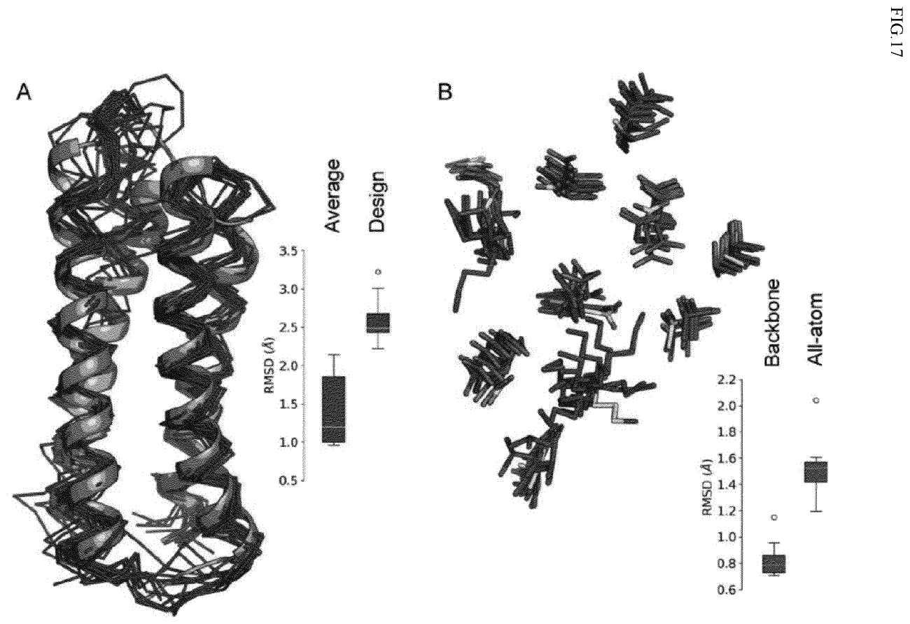

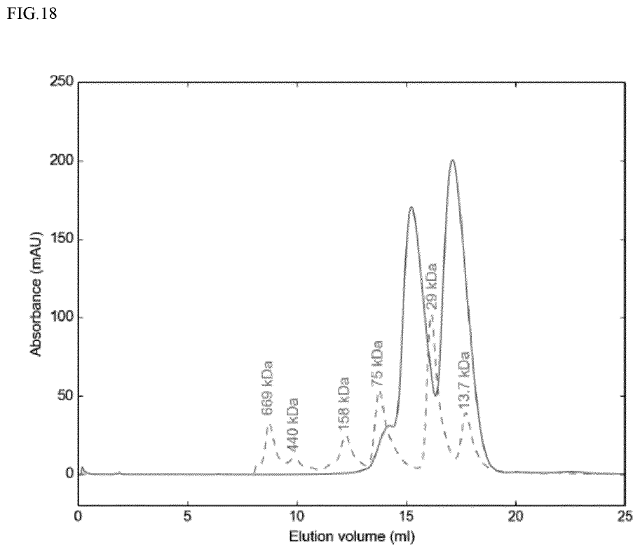

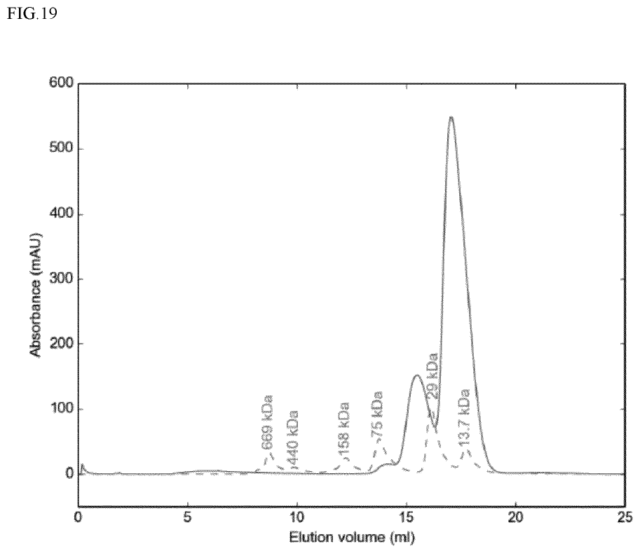

To characterize the kinetics and affinity of interactions between the designs and the G-CSF receptor, the inventors performed surface plasmon resonance (SPR)-based measurements for Boskar3 and Boskar4 in comparison to rhG-CSF. Analysis of the kinetics across the injection dilution series, assuming 1:1 binding, resulted in dissociation constants (Kd) of 14 nM and 5.1 nM for Boskar3 and Boskar4, respectively. In comparison, the Kd determined for rhG-CSF was 335 μM (Table 9). Previous studies have reported Kd values for the G-CSF:G-CSFR interaction between 200 μM using SPR [38] and 1.4 nM using ITC [39]. To obtain a more detailed picture on the nature of the binding, the inventors fitted the highest concentration sensorgram curves using higher order kinetics models. These fitting attempts showed the second-order reaction model to better fit the data than a first-order model despite the same number of parameters in each model. This indicates that the binding reaction depends on two analyte molecules, yielding Kd values of 4.4 μM, 6.1 μM, 86 nM, for Boskar3, Boskar4, and rhG-CSF, respectively. While this higher-order interaction model better explains the data than a 1:1 binding model, a clear deviation remained for rhG-CSF sensorgrams. While this may point to different interaction modes between the two designs and rhG-CSF with the G-CSFR, it demonstrates that the binding form of the designs is plausibly dimeric. Size-exclusion chromatography of the designs indeed show that the designs partition between monomeric and dimeric forms (FIGS. 18 and 19)).

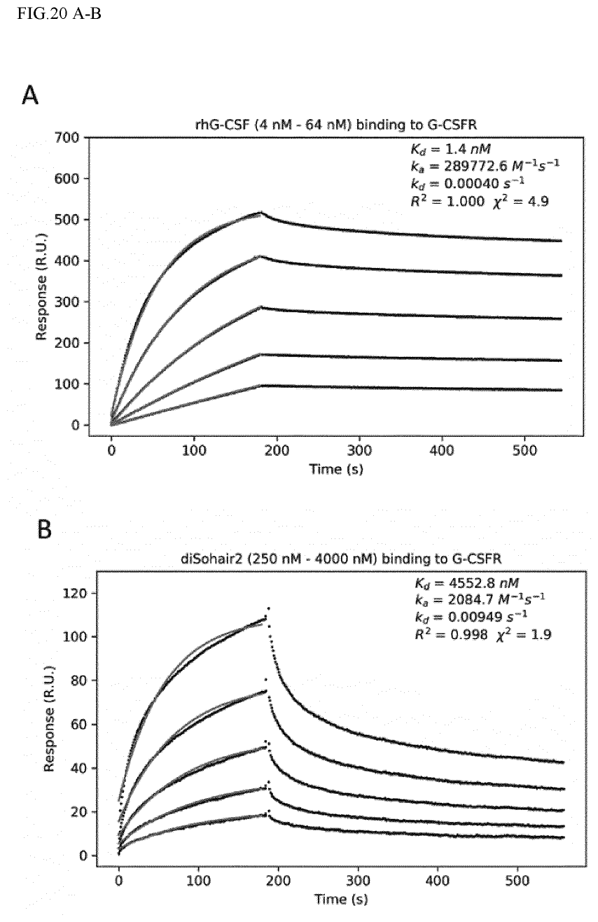

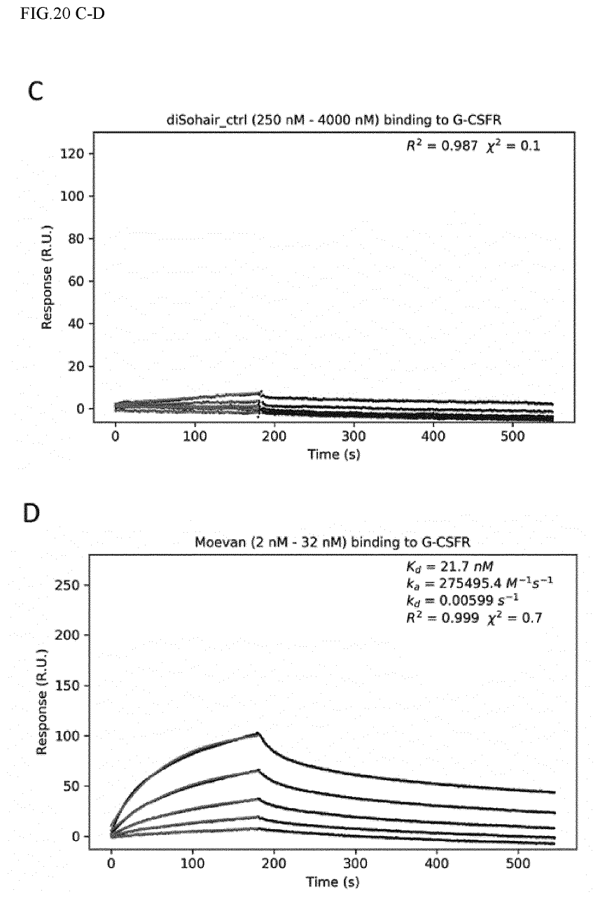

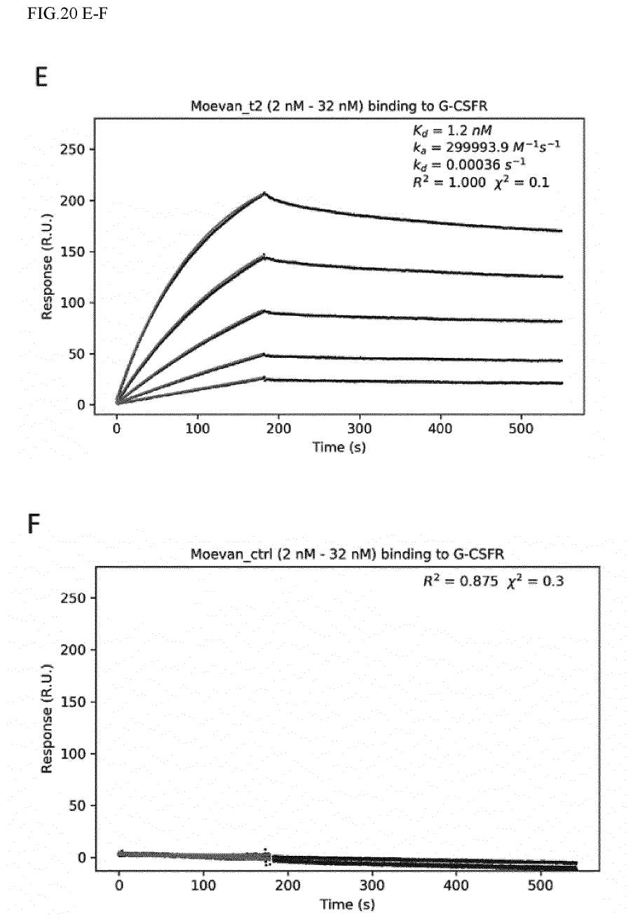

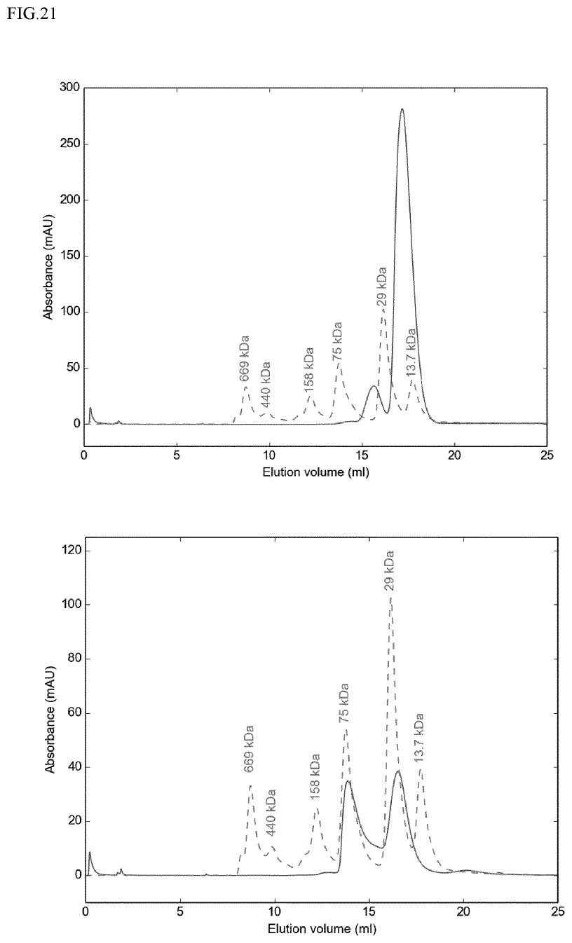

To characterize the kinetics and affinity of interactions between the designs and the G-CSF receptor, the inventors performed surface plasmon resonance-based measurements for Moevan and diSohair2 in comparison to rhG-CSF (Table 10). Analysis of the kinetics across the injection dilution series, assuming 1:1 binding, resulted in dissociation constants (Kd) of 4.5 μM, 21.0 nM, and 1.1 nM for diSohair2 (FIG. 20B), Moevan (FIG. 20D), and Moevan_t2 (FIG. 20E), respectively. In comparison, the Kd determined for rhG-CSF was 1.1 nM (FIG. 20A), in line with previous studies that have reported Kd values for the G-CSF:G-CSFR interaction between 200 μM using SPR [38] and 1.4 nM using ITC [39]. To test whether the grafted epitope residues mediate binding of the designs to the G-CSF receptor, the inventors also performed SPR measurements for the Moevan and diSohair2 initial design templates (Moevan_control and diSohair_control, respectively), and no binding was observed (FIG. 20C,F). As bivalency influences the binding to and the activation of the G-CSFR, the inventors also performed analytical size exclusion chromatography, which showed that diSohair2 assumes both dimeric and tetrameric forms, whereas Moevan is majorly monomeric with a minor dimeric fraction (FIG. 21).

The Moevan control and diSohair control refers to the unmutated scaffold protein sequences of both diSohair2 (PDB: 5J73) and Moevan (PDB: 2QUP), lacking the G-CSF-R binding epitope.

Methods:

Single-cycle kinetics experiments were performed on a Biacore X100 system (GE Healthcare Life Sciences). G-CSF Receptor (G-CSFR) (R&D systems 381-GR-050/CF) was diluted to 50 μg/ml in 10 mM acetate buffer pH 5.0 and immobilized on the surface of a CM5 sensor chip (GE Healthcare 29149604) using standard amine coupling chemistry. The designs and rhG-CSF (USP RS Filgrastim, Sigma-Aldrich 1270435) were diluted in running buffer (10 mM HEPES, 150 mM NaCl, 3.4 mM EDTA, 0.005% v/v Tween-20). Analyses were conducted at 25° C. at a flow rate of 30 μl/min. Five sequential 10-fold increasing concentrations of the sample solution (for the designs from 0.5 nM to 50 μM, and for rhG-CSF from 0.05 to 500 nM) were injected over the functionalized sensor chip surface for 180 s, followed by a 180 s dissociation with running buffer. At the end of each run, the sensor surface was regenerated with a 240 s injection of 10 mM glycine-HCl pH 2.0. Each experiment was performed two times for rhG-CSF, Boskar3, Boskar4, diSohair2, Moevan, and Moevan_t2. Association rate (ka), dissociation rate (kd), and equilibrium dissociation (Kd) constants were initially obtained by global fitting of the experimental reference-subtracted data to a 1:1 interaction model using the Biacore X100 evaluation software (v.2.0.1). To evaluate if a kinetics model that depends on double the analyte stoichiometry improves the goodness of fit to the data, the following rate integral was used:

$$R ( t ) = \{ \begin{matrix}{R}_{max} - \frac{1}{\frac{1}{{R}_{max}} + {k}_{a} Ct} & if 0 < t \leq 180 \\ \frac{1}{\frac{1}{{R}_{max}} + {k}_{d} t} & if 180 < t \leq 360\end{matrix}$$

where R(t) is the normalized response at time tin normalized response units (and time t is in seconds), and Rmax is the maximum normalized response (i.e. R(180 s)), at analyte concentration C, given association and dissociation intervals of 180 s each. The goodness of fit was evaluated by the χ2 as:

$${𝒳}^{2} = \frac{\sum {( {R}_{fit} - {R}_{obs} )}^{2}}{n}$$

where Rfit is the R(t) function with minimum sum of square deviation from the observed sensorgram curve Robs, optimizing ka and kd, individually, within the bounds [10, 1×10⁶] and [1×10⁻⁵, 0.1], respectively. The optimization was performed using the Nelder-Mead method at a tolerance of 1×10⁻⁸ and a maximum number of iterations of 1×10⁴. The coefficient of determination R² was calculated as:

$${R}^{2} = 1 - \frac{\sum {( {R}_{fit} - {R}_{obs} )}^{2}}{\sum {( {R}_{obs} - 〈 {R}_{obs} 〉 )}^{2}}$$

where <⋅> is the vector average.

### Example 12: Activation of G-CSFR Signaling by Boskar3 and Boskar4

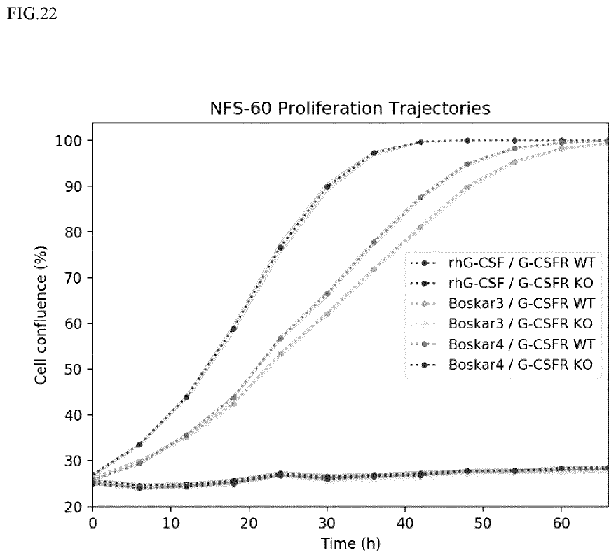

To evaluate the dependency of the response to the designed proteins on G-CSFR expression, the inventors knocked out G-CSFR in NFS-60 cells using CRISPR/Cas9-mediated mutagenesis. For this, the inventors synthesized guide RNA (gRNA) specifically targeting exon 4 of CSF3R (cut site: chr4 [+126,029,810: −126,029,810]) to introduce stop-codon or frameshift mutations in the extracellular part of all G-CSFR isoforms. The inventors generated pure G-CSFR KO NFS-60 cell clones that have one nucleotide deletion on each allele, as assessed by Sanger sequencing and tracking of indels by decomposition (TIDE) analysis. In contrast to wild type cells, G-CSFR KO NFS-60 cells did not respond to treatment with rhG-CSF, Boskar3 or Boskar4 (FIG. 22). These data demonstrate that the designed proteins act via G-CSFR.

Methods:

A specific guide RNA (sgRNA) for knock-out of the CSF3R gene (cut site: chr4 [+126.029.810: −126.029.810], NM_007782.3 and NM_001252651.1, exon 4, 112 by after ATG; NP_031808.2 and NP_001239580.1 p.L38) was designed using CCTop at (http://crispr.cos.uni-heidelberg.de) [54]. Electroporation of NFS-60 cells was carried out using the Amaxa nucleofection system (SF cell line 4D-Nucleofector kit, #V4XC-2012) according to the manufacturer's instructions. Briefly, 1×106 cells were electroporated with assembled sgRNA (8 μg) and HiFi Cas9 nuclease protein (15 μg) (Integrated DNA Technologies). Clonal isolation of single-cell derived NFS-60 cells was performed by limiting dilution followed by an expansion period of 3 weeks. Genomic DNA of each single-cell derived NFS-60 clones was isolated using QuickExtract DNA extraction solution (Lucigen #QE09050). PCR was carried out with mouse CSF3R-specific primers (forward: 5′-GGCATTCACACCATGGGGCACA-3′, reverse: 5′-GCCTGCGTGAAGCTCAGCTTGA-3′) and the GoTaq Hot Start Polymerase Kit (Promega, #M5006) using 2 μl of gDNA template for each PCR reaction. In vitro cleavage assay was done by adding 1 μM Cas9 RNP assembled by the same sgRNA used for the knock-out experiment to 3 μL of each PCR product. The PCR reactions were incubated at 37° C. for 60 min and run on a 1% agarose gel. The PCR products that showed no cleavage were purified by ExoSAP (ratio 3:1), which is a master mix of one-part Exonuclease I 20 U/μl (Thermo Fisher Scientific, #EN0581) and two parts of FastAP thermosensitive alkaline phosphatase 1 U/μl (Thermo Fisher Scientific, #EF0651). Sanger sequencing of purified PCR products was performed by Microsynth and analysed using the TIDE (Tracking of Indels by Decomposition) webtool.

### Example 13: Activation of G-CSF Receptor Downstream Intracellular Signaling Pathways in Human Hematopoietic Stem Cells

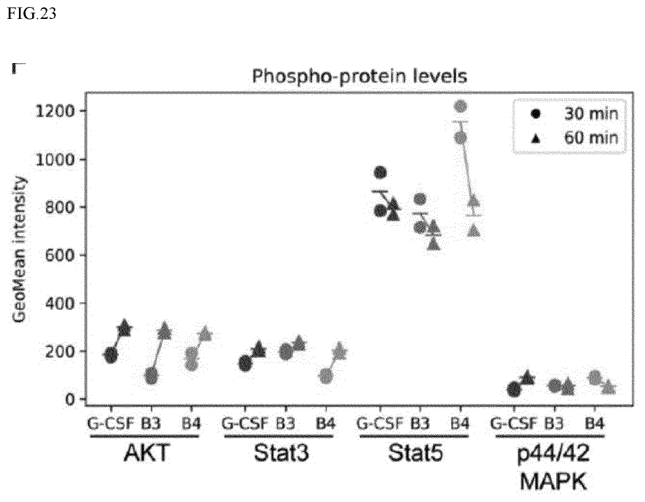

Binding of G-CSF to G-CSFR rapidly activates a cascade of intracellular events, including phosphorylation of downstream effectors, e.g. Akt, STAT3, STAT5 or MAPK, that ultimately induce granulocytic differentiation. To test whether our designed proteins directly induce G-CSFR signaling, the inventors measured these immediate phosphorylation targets of G-CSFR signaling in CD34+ HSPCs. Indeed, the inventors found that Akt, STAT3, STAT5 and p44/42 MAPK (Erk1/2) were tyrosine phosphorylated in HSPCs treated with Boskar3 or Boskar4 to a similar degree as in rhG-CSF-treated cells (FIG. 23). Together, this shows that the biological activity of the designs is directly attributable to G-CSFR activation.

Methods:

CD34+ 703 cells were cultured in Stemline II Hematopoietic Stemcell Expansion Medium (Sigma-Aldrich; #50192) supplemented with 10% FBS (Sigma-Aldrich; #F7524), 1% L-glutamine (Biochrom; #K0283), 1% penicillin/streptomycin (Biochrom; #A2213) and a premixed cytokine cocktail containing IL-3 (PeproTech; #200-03), IL-6 (Novus Biologicals; #NBP2-34901), TPO (R&D Systems; #288-TP200), rhSCF (R&D Systems; #255-SC-200) and Flt-3L (BioLegend; #550606). Final concentrations were 20 ng/ml for IL-3, IL-6 and TPO, and 50 ng/ml for SCF and Flt-3L. On day 6 of culture, serum- and cytokine-starved (4 h) CD34+ HSPCs were treated with 20 ng/ml of rhG-CSF, 10 μg/ml of Boskar3 or 10 μg/ml of Boskar4 for 30 or 60 min, fixed in 4% PFA (Merck; #P6148) for 15 min at room temperature, and permeabilised by slowly adding ice-cold methanol (C. Roth; #7342.1) to a final concentration of 90% and incubating for 30 min. Cells were left overnight in methanol at −20° C. and stained on the next day by incubation for 20 min on ice in PBS/2% BSA with specific antibodies recognizing the phosphorylated signaling effectors, phospho-Stat3 (Tyr705) (D3A7) XP rabbit mAb (Cell Signaling; #9145); phospho-Stat5 (Tyr694) (C1105) rabbit mAb (Cell Signaling; #9359); phospho AKT (Thr308) (244F9) rabbit mAb (Cell Signaling; #4056S), and phospho-p44/42 MAPK (Erk1/2) (Thr202/Tyr204) (E10) mouse mAb (Cell Signaling; #9106), or the respective Alexa Fluor 488-conjugated isotype control antibody, anti-mouse IgG (H+L) F(ab′)2 fragment (Cell Signaling; #4408) or goat anti-rabbit IgG H+L (Abcam; #ab150077). Thereafter, cells were washed twice in ice-cold PBS/2% BSA and analyzed by FACS. The background-corrected fluorescence signal was distinguished from the corresponding phosphorylated proteins by subtracting the fluorescence signal of the appropriate isotype control, estimated at each time point of stimulation, from the specific phospho-protein signal.

### Example 14: Neutrophils Generated from Design-Treated HSPCs are Functional

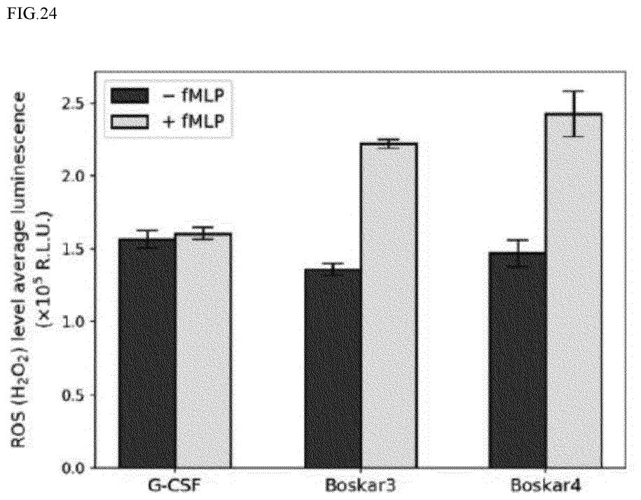

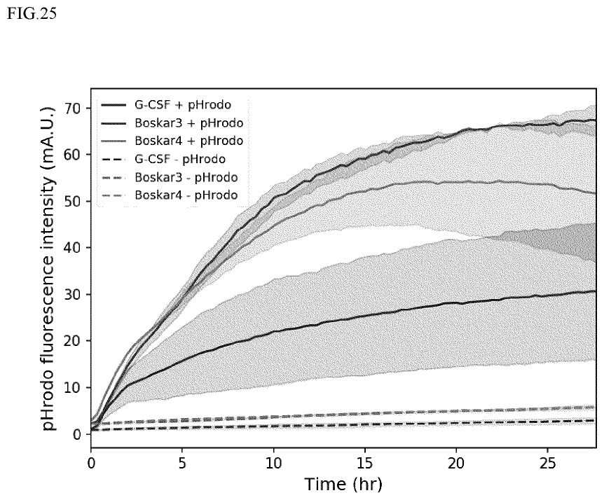

To test whether the neutrophils differentiated by our designs can execute neutrophil-specific functions such as production of reactive oxygen species (ROS) and phagocytosis, the inventors evaluated in vitro activation of neutrophils generated from Boskar3- and Boskar4-treated HSPCs in liquid culture for 14 days. For that, cells were seeded at a density of 1×10⁵ cells/mL with or without 10 nM tMLP (Sigma, #F3506) and incubated for 30 min at 37° C. and 5% CO₂. The level of hydrogen peroxide (H₂O₂), a reactive oxygen species (ROS), was measured with the ROS-Glo H₂O₂ Assay kit (Promega, #G8820) according to the manufacturer's protocol. The inventors first assessed H₂O₂ levels in N-Formylmethionyl-leucyl-phenylalanine (fMLP)-activated neutrophils and detected even higher ROS levels in Boskar-generated neutrophils compared to rhG-CSF-stimulated samples (FIG. 24). Phagocytosis was evaluated using live cell imaging of neutrophils incubated with pHrodo Green E. coli bioparticles. The inventors observed similar phagocytosis behavior of rhG-CSF- and Boskar-generated neutrophils (FIG. 25). These data show that our designed proteins induce functionally active neutrophils.

Methods:

Granulocytes from day 14 of liquid culture differentiation were cultured in RPMI 1640 medium supplemented with 0.5% BSA and pHrodo Green E. coli Bioparticles Conjugate (Essen Bio; #4616) according to the manufacturer's protocol (Essen Bio) at 37° C. and 5% CO2. Briefly, 1×10⁴ cells were seeded in 90 μl medium, and 10 μg of Bioparticles were added to a final volume of 100 μl. The cells were monitored for 8 h in an IncuCyte S3 Live-Cell Analysis System (Essen Bio) with a 10× objective. The analysis was conducted in IncuCyte S3 Software.

### Example 15: The Designed Proteins Induce Myeloid Differentiation of HSPCs in Mice

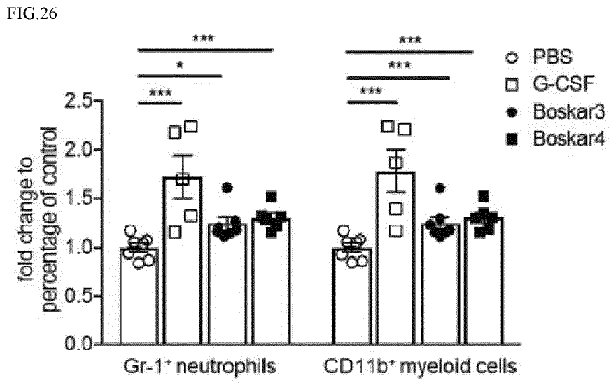

The inventors next evaluated the effects of the designed proteins on the proliferation and myeloid differentiation of HSPCs in mice. The inventors treated C57BL/6 mice with rhG-CSF or G-CSF designs, Boskar3 and Boskar4 at a concentration of 300 μg/kg by intraperitoneal injection (i.p.) every second day for a total of three injections. Mice in the control group were treated with PBS using the same treatment scheme. Two days after the third injection, the number of CD11b+ myeloid cells and of Gr-1+ 311 neutrophilic granulocytes in the bone marrow of treated mice was evaluated. The inventors found that treatment of mice with rhG-CSF, Boskar3, or Boskar4 induces production of myeloid cells and neutrophils, as compared to the control PBS-treated group (FIG. 26). No toxic effects of the designed proteins were observed. These results demonstrate the granulopoietic activity of our designed proteins in vivo.

Methods:

C57BL/6 mice (The Jackson Laboratory) were maintained under pathogen-free conditions in the research animal facility of the University of Tübingen, according to German federal and state regulations (Regierungspräsidium Tübingen, K3/17). Mice were treated with intraperitoneal injections (i.p.) of rhG-CSF, Boskar3, or Boskar4 at a concentration of 300 μg/kg every second day for a total of three injections. Mice were sacrificed 2 days after the last injection. Mice in the control group were treated with PBS using the same schema. Bone marrow cells were isolated by flushing with a 22 G syringe, and filtered through a 0.45 μm cell strainer prior to counting and staining for flow cytometry analyses. For the analysis of Gr-1+ or CD11b+ myeloid cells, 0.5×10⁶ cells were transferred into FACS tubes and washed once with FACS buffer. Phycoerythrin (PE)-Cyanine7-conjugated anti-mouse Ly-6G/Ly-6C (Gr-1) antibody (clone RB6-8C5; eBioscience) or PE-conjugated anti-mouse CD11 b antibody (clone M1/70; BioLegend) was added to a final concentration of 1-5 μg/ml according to the manufacturer's instructions, and cells were incubated in the dark at 4° C. for 30 min. Thereafter, cells were washed twice with ice-cold FACS buffer. All centrifugation steps were conducted at 400×g, 4° C. for 5 min. Samples were measured on a LSR II cytometer and analyzed using BD FACSDiva software. For all FACS analyses, vital mononuclear cells were selected, and doublets were excluded based on scatter characteristics.

### REFERENCES REFERRED TO HEREIN ABOVE

[1] Kinch, M. S., An overview of FDA-approved biologics medicines. Drug Discovery Today, 2015. 20(4): p. 393-398.
[2] Kintzing, J. R., M. V. Filsinger Interrante, and J. R. Cochran, Emerging Strategies for Developing Next-Generation Protein Therapeutics for Cancer Treatment. Trends in Pharmacological Sciences, 2016. 37(12): p. 993-1008.
[3] Zídek, Z., P. Anzenbacher, and E. Kmoničková, Current status and challenges of cytokine pharmacology. British Journal of Pharmacology, 2009. 157(3): p. 342-361.
[4] Platanias, L. C., Mechanisms of type-I- and type-II-interferon-mediated signalling. Nature Reviews Immunology, 2005. 5: p. 375.
[5] Dale, D. C., et al., Review: Granulocyte Colony-Stimulating Factor—Role and Relationships in Infectious Diseases. The Journal of Infectious Diseases, 1995. 172(4): p. 1061-1075.
[6] Dale, D. C., et al., A systematic literature review of the efficacy, effectiveness, and safety of filgrastim. Supportive Care in Cancer, 2018. 26(1): p. 7-20.
[7] Kuwabara, T., S. Kobayashi, and Y. Sugiyama, Pharmacokinetics and Pharmacodynamics of a Recombinant Human Granulocyte Colony-Stimulating Factor. Drug Metabolism Reviews, 1996. 28(4): p. 625-658.
[8] Arvedson, T., J. O'Kelly, and B.-B. Yang, Design Rationale and Development Approach for Pegfilgrastim as a Long-Acting Granulocyte Colony-Stimulating Factor. Biodrugs, 2015. 29(3): p. 185-198.
[9] Bishop, B., et al., Reengineering Granulocyte Colony-stimulating Factor for Enhanced Stability. Journal of Biological Chemistry, 2001. 276(36): p. 33465-33470.
[10] Miyafusa, T., et al., Backbone Circularization Coupled with Optimization of Connecting Segment in Effectively Improving the Stability of Granulocyte-Colony Stimulating Factor. ACS Chemical Biology, 2017. 12(10): p. 2690-2696.
[11] Vanz, A. L. S., et al., Human granulocyte colony stimulating factor (hG-CSF): cloning, overexpression, purification and characterization. Microbial Cell Factories, 2008. 7(1): p. 13.
[12] Zink, T., et al., Structure and Dynamics of the Human Granulocyte Colony-Stimulating Factor Determined by NMR Spectroscopy. Loop Mobility in a Four-Helix-Bundle Protein. Biochemistry, 1994. 33(28): p. 8453-8463.
[13] Hill, C. D., et al., The structure of granulocyte-colony-stimulating factor and its relationship to other growth factors. Proc Natl Acad Sci USA, 1993. 90(11): p. 5167-5171.
[14] Schneider, A., et al., The hematopoietic factor G-CSF is a neuronal ligand that counteracts programmed cell death and drives neurogenesis. J Clin Invest, 2005. 115(8): p. 2083-2098.
[15] England, T. J., et al., Granulocyte-Colony Stimulating Factor (G-CSF) for stroke: an individual patient data meta-analysis. Sci Rep, 2016. 6: 36567.
[16] Sanchez-Ramos, J., et al., Pilot study of granulocyte-colony stimulating factor for treatment of Alzheimer's disease. J Alzheimers Dis, 2012. 31(4): p. 843-855.
[17] Altschul, S. F., et al., Basic local alignment search tool. J Mol Biol, 1990. 215(3): p. 403-410.
[18] Carter, C. R. D., et al., The significance of carbohydrates on G-CSF: differential sensitivity of G-CSFs to human neutrophil elastase degradation. Journal of Leukocyte Biology, 2004. 75(3): p. 515-522.
[19] μl Ouriaghli, F., et al., Neutrophil elastase enzymatically antagonizes the in vitro action of G-CSF: implications for the regulation of granulopoiesis. Blood, 2003. 101(5): p. 1752.
[20] Plaxco, K. W., et al., Contact order, transition state placement and the refolding rates of single domain proteins. J Mol Biol, 1998, 277(4): p. 985-994.
[21] Liles, W. C., Augmented mobilization and collection of CD34+ hematopoietic cells from normal human volunteers stimulated with granulocyte colony-stimulating factor by single administration of AMD3100, a CXCR-4 antagonist. Transfusion, 2005, 45: p. 295-300.
[22] Flomemberg, N., et al., The use of AMD3100 plus G-CSF for autologous hematopoietic progenitor cell mobilization is superior to G-CSF alone. Blood, 2005, 106: p. 1867-1874.
[23] Broxmeyer, H. E., et al., Rapid mobilization of murine and human hematopoietic stem and progenitor cells with AMD3100, a CXCR-4 antagonist. J Exp Med, 2005, 201: p. 1307-1318.
[24] Devine, S. M., et al., A pilot study evaluating the safety and efficacy of AMD3100 for the mobilization and transplantation of HLA-matched sibling donors hematopoietic stem cells in patients with advanced hematological malignancies. Blood, 2005, 106: p. 299-304.
[25] Raso, S. W., et al., Aggregation of granulocyte-colony stimulating factor in vitro involves a conformationally altered monomeric state. Protein Science, 2005, 14(9): p. 2246-2257.
[26] Young, D. C., et al., Characterization of the receptor binding determinants of granulocyte colony stimulating factor. Protein Sci, 2997, 6(6): p. 1228-1236
[27] Layton, J. E., et al., Interaction of Granulocyte Colony-stimulating Factor (G-CSF) with its receptor: evidence that Glu¹⁹ of G-CSF interacts with Arg²⁸⁸ of the receptor. J Biol Chem, 1999, 274(25): p. 17445-17451.
[28] Silva, D. A., et al., De novo design of potent and selective mimics of IL-2 and IL-15. Nature, 2019, 565, p. 186-191.
[29] Jones, D. T., Protein secondary structure prediction based on position-specific scoring matrices. J Mol Biol, 1999, 292, p. 195-202.
[30] Yang, Y., et al., SPIDER2: A Package to Predict Secondary Structure, Accessible Surface Area, and Main-Chain Torsional Angles by Deep Neural Networks. Methods Mol Biol, 2017, 1484, p. 55-63.
[31] Wang, S., et al., DeepCNF-SS: Protein Secondary Structure Prediction Using Deep Convolutional Neural Fields. Sci Rep, 2016, 6, 18962.
[32] Lupas, A., et al., Predicting coiled coils from protein sequences. Science, 1991, 252, p. 1162-1164.
[33] Czekanska, E. M., Assessment of cell proliferation with resazurin-based fluorescent dye. Methods Mol Biol, 2011, 740, p. 27-32.
[34] Kabsch, W., A discussion of the solution for the best rotation to relate two sets of vectors. Acta Cryst, 1978, 34, p. 827-828.
[35] Skokowa, J., et al., Neutrophil elastase is severely down-regulated in severe congenital neutropenia independent of ELA2 or HAX1 mutations but dependent on LEF-1. Blood, 2009, 114, p. 3044-3051.
[36] Velázquez-Campoy, A., et al., Isothermal Titration Calorimetry. Current Protocols in Cell Biology, 2004, 23, 17.8.1-17.8.24.
[37] ElGamacy, M., et al., An Interface-Driven Design Strategy Yields a Novel, Corrugated Protein Architecture, ACS Synthetic Biology, 2018, 7 (9), 2226-2235.
[38] Heinzelmann, P., et al., pH responsive granulocyte colony-stimulating factor variants with implications for treating Alzheimer's disease and other central nervous system disorders. Protein engineering, design & selection: PEDS, 2015. 28(10), 481-489.
[39] Mine, S., et al., Thermodynamic Analysis of the Activation Mechanism of the GCSF Receptor Induced by Ligand Binding. Biochemistry, 2004. 43(9), 2458-2464.
[40] Luo, P., et al., Development of a cytokine analog with enhanced stability using computational ultrahigh throughput screening. Protein Sci, 2002. 11(5), 1218-1226.

The application text refers to the following tables:

TABLE 1
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gin (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; | Tyr |
| | Ile; Ala; Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

TABLE 2
Sequence identities of the protein variants
of the invention with human G-CSF

| | | Sequence |
|---|---|---|
| | | identity with G- |
| | Highest local | CSF over the |
| | sequence identity | whole length |
| Protein design | with G-CSF | of the protein |
| Boskar_1 | 53 identical residues over 119 | 43% |
| (SEQ ID NO: 2) | residues of Boskar_1 (45% | |
| | identity) | |
| Boskar_2 | 52/119 | 42% |
| (SEQ ID NO: 3) | (44%) | |
| Boskar_3 | 55/119 | 45% |
| (SEQ ID NO: 4) | (46%) | |
| Boskar_4 | 51/119 | 42% |
| (SEQ ID NO: 5) | (43%) | |
| Moevan | 15/22(68%) | 13% |
| (SEQ ID NO: 6) | | |
| Moevan_es1.1 | 14/22(64%) | 12% |
| (SEQ ID NO: 7) | | |
| Moevan_es1.2 | 14/22(64%) | 12% |
| (SEQ ID NO: 8) | | |
| Moevan_es1.3 | 14/22(64%) | 12% |
| (SEQ ID NO: 9) | | |
| Moevan_ea1.1 | 12/22(55%) | 10% |
| (SEQ ID NO: 10) | | |
| Moevan_ea1.2 | 12/22(55%) | 10% |
| (SEQ ID NO: 11) | | |
| Moevan_ea1.3 | 12/22(55%) | 10% |
| (SEQ ID NO: 12) | | |
| Moevan_ea1.4 | 12/22(55%) | 10% |
| (SEQ ID NO: 13) | | |
| Moevan_ea2.5 | 11/18(61%) | 13% |
| (SEQ ID NO: 20) | | |
| Moevan_ea2.6 | 11/18(61%) | 12% |
| (SEQ ID NO: 21) | | |
| Moevan_ea2.7 | 11/19 | 12% |
| (SEQ ID NO: 22) | (58%) | |
| Sohair | 11/23 | 7% |
| (SEQ ID NO: 14) | (48%) | |
| Sohair_esa1.1 | No significant similarity found | |
| (SEQ ID NO: 15) | | |
| Sohair_esa1.2 | 3/5(60%) | 4% |
| (SEQ ID NO: 16) | | |
| Sohair_esa1.3 | 8/20(40%) | 7% |
| (SEQ ID NO: 17) | | |
| Sohair_esa2.4 | 9/20(45%) | 8% |
| (SEQ ID NO: 23) | | |
| Sohair_esa2.5 | 8/19(42%) | 7% |
| (SEQ ID NO: 24) | | |
| Sohair_esa2.6 | No significant similarity found | |
| (SEQ ID NO: 25) | | |
| Disohair_1 | 11/23 | 15% |
| (SEQ ID NO: 18) | (48%) | |
| Disohair_2 | 11/23 | 15% |
| (SEQ ID NO: 19) | (48%) | |

TABLE 3
Amino acid residues involved in α-helices according to
design models and G-CSF crystal structure (2D9Q).

| Protein | | | | | Total |
|---|---|---|---|---|---|
| design | Helix 1 | Helix 2 | Helix 3 | Helix 4 | length |
| G-CSF | 11-37 | 74-90 | 101-122 | 143-171 | 174 |
| Boskar_4 | 2-22 | 27-53 | 60-87 | 92-116 | 119 |
| Moevan | 3-33 | 36-63 | 71-93 | 99-117 | 118 |
| Sohair | 4-37 | 41-75 | 82-114 | 119-152 | 154 |
| Disohair_2 | 4-37 | 41-75 | 4-37 | 41-75 | 76 |
| Bika1 | 2-32 | 39-62 | 2-32 | 39-62 | 64 |

TABLE 4
Absolute contact orders of protein variants

| Protein design | Absolute Contact Order |
|---|---|
| G-CSF | 18.60 |
| Boskar_4 | 17.84 |
| Moevan | 9.42 |
| Sohair | 4.53 |
| Disohair_2 | 4.53 |

TABLE 5
Amino acid sequences and EC50 for activating the proliferation of NFS-60
cells. The residues highlighted in grey are involved in the binding to the
G-CSF receptor.

| | NFS-60 EC50 |
|---|---|
| Sequence | (ng/mL) |
| >boskar_1 (SEQ ID NO: 2) | 2173 |
| AALAAELAEIYKGLAEYQARLQSLEGISPELGPALDALRL [symbol] VA [symbol] FA | |
| TTLAQAMEEKKTNLPQSFLL [symbol] AL [symbol] I [symbol] IQA [symbol] AAALREKLAATYTG | |
| TDRAAAAVEIAAQLEAFLEKAYEILRHLAAA | |
| >boskar_2 (SEQ ID NO: 3) | 3225 |
| AALAAELAEIMKGLQEYQARLKSLEGISPELGPALDALRL [symbol] MA [symbol] FA | |
| TTMAQMMEENPSDLPQSFLL [symbol] AL [symbol] I [symbol] IQA [symbol] AAALREKLAATYP | |
| NSQRAAAAVEIAAQLEAFLEKAYQILRHLAAA | |
| >boskar_3 (SEQ ID NO: 4) | 768 |
| AALAAVLAEIYKGLAEYQARLQSLEGISPELGPALDALRL [symbol] VA [symbol] FA | |
| TTIAQAMEENKGPLPQSFLL [symbol] AL [symbol] I [symbol] IQA [symbol] AAALREKLAATYPSS | |
| QRAAAAVEIAAQLEAFLEKAYEILRHLAAA | |
| >boskar_4 (SEQ ID NO: 5) | 27 |
| AALAAALAEIYKGLAEYQARLKSLEGISPELGPALDALRL [symbol] MA [symbol] FA | |
| TTMAQAMEEGLDSLPQSFLL [symbol] AL [symbol] I [symbol] IQA [symbol] AAALREKLAATYKG | |
| NDRAAAAVEIAAQLEAFLEKAYQILRHLAAA | |
| >moevan (SEQ ID NO: 6) | 356 |
| MEAAAAARDESAYL [symbol] LQ [symbol] M [symbol] IDA [symbol] AAALSETRTIEELDTFKL [symbol] VA [symbol] FV | |
| TTVVQLAEELEHRFGRNRRGRTEIYKIVKEVDRKLLDLTDAVLAKEKKGE | |
| DILNMVAEIKALLINIYK | |
| >disohair_1 (SEQ ID NO: 18) | 2375 |
| MTSDYIIEQIQRKQEEARL [symbol] VE [symbol] ME [symbol] LEEVKEASKRGVSSDQLLNLIL [symbol] L | |
| A [symbol] IITTLIQIIEESNEAIKELIKNQ | |
| >disohair_2 (SEQ ID NO: 19) | 396 |
| MTSDYIIEQIQRKQEEARL [symbol] VE [symbol] E [symbol] LEAVKEASKRGVSSDQLLNLIL [symbol] L | |
| A [symbol] IITTLIQIIEESNEAIKELIKNQ | |
| >sohair (SEQ ID NO: 14) | 5053 |
| MTSDYIIEQIQRKQEEARL [symbol] VE [symbol] ME [symbol] LEAVKEASKRGVSSDQLLNLIL [symbol] L | |
| A [symbol] IITTLIQIIEESNEAIKELIKNQKGPTSDYIIEQIQRDQEEARKKVEEAEER | |
| LERVKEASKRGVSSDQLLDLIRELAEIIEELIRIIRRSNEAIKELIKNQ | |
| >csf_2d9q\|WILD_TYPE G-CSF | 0.055 |
| MSSLPQSFLL [symbol] CL [symbol] V [symbol] IQG [symbol] GAALQEKLCATYKLCHPEELVLLGHSL | |
| GIPWAPLSSCPSQALQLAGCLSQLHSGLFLYQGLLQALEGISPELGPTLD | |
| TLQL [symbol] VA [symbol] FATTIWQQMEELGMAPALQPTQGAMPAFASAFQRRAGGVL | |
| VASHLQSFLEVSYRVLRHLAQP | |

TABLE 6
Comparison of the protein designs with recombinant human G-CSF

| | Chain | | | | | |
|---|---|---|---|---|---|---|
| | length | Yield | | | Thermal | |
| | (amino | (per litre | | Contact- | stability | Protease |
| Protein | acids) | culture) | Solubility | order | (Tₘ) | resistance |
| Moevan | 118 | >15 mg/L | <5 mg/mL | 9.42 | 74° C. | − |
| (SEQ ID | | | | | | |
| NO: 6) | | | | | | |
| DiSohair_2 | 76 | >30 mg/L | >30 mg/mL | 4.53 | >100° C. | +++ |
| (SEQ ID | | | | | | |
| NO: 19) | | | | | | |
| Boskar_4 | 119 | >30 mg/L | >15 mg/mL | 17.84 | >100° C. | +++ |
| (SEQ ID | | | | | | |
| NO: 5) | | | | | | |
| rhG-CSF | 174 | 3.2 mg/L | <4 mg/mL | 18.60 | 57° C. | − |
| (SEQ ID | | | | | | |
| NO: 1) | | | | | | |

TABLE 7
Amino acid sequences and EC50 for activating the proliferation of NFS-60
cells The residues highlighted in grey are involved in the binding to the
G-CSF receptor.

| | NFS-60 EC₅₀ |
|---|---|
| Sequence | (ng/mL) |
| >boskar4_t2 (SEQ ID NO: 26) | 4.2 |
| AALAAALAEIYKGLAEYQARLKSLEGISPELGPALDALRL [symbol] M [symbol] FA | |
| TTMAQAMEEGLDSLPQSFLL [symbol] AL [symbol] I [symbol] IQA [symbol] AAALREKLAATYKG | |
| NDRAAAAVEIAAQLEAFLEKAYQILRHLAAA | |
| GGGGSSGGGGSSGGGGSSGGGGSS | |
| AALAAALAEIYKGLAEYQARLKSLEGISPELGPALDALRL [symbol] MA [symbol] FA | |
| TTMAQAMEEGLDSLPQSFLL [symbol] AL [symbol] I [symbol] IQA [symbol] AAALREKLAATYKG | |
| NDRAAAAVEIAAQLEAFLEKAYQILRHLAAA | |
| >boskar4_st2 (SEQ ID NO: 27) | 0.202 |
| AALAAALAEIYKGLAEYQARLKSLEGISPELGPALDALRL [symbol] MA [symbol] FA | |
| TTMAQAMEEGLDSLPQSFLL [symbol] AL [symbol] I [symbol] IQA [symbol] AAALREKLAATYKG | |
| NDRAAAAVEIAAQLEAFLEKAYQILRHLAAA | |
| GGGGSS | |
| AALAAALAEIYKGLAEYQARLKSLEGISPELGPALDALRL [symbol] MA [symbol] FA | |
| TTMAQAMEEGLDSLPQSFLL [symbol] AL [symbol] I [symbol] IQA [symbol] AAALREKLAATYKG | |
| NDRAAAAVEIAAQLEAFLEKAYQILRHLAAA | |
| >boskar4_15rl (SEQ ID NO: 28) | 48.5 |
| AALAAALAEIYKGLAEYQARLKSLEGISPELGPALDALRL [symbol] MA [symbol] FA | |
| TTMAQAME | |
| GGGGSGGGGSGGGGS | |
| QSFLL [symbol] AL [symbol] I [symbol] IQA [symbol] AAALREKLAATYKGNDRAAAAVEIAAQLE | |
| AFLEKAYQILRHLAAA | |
| >moevan_t2 (SEQ ID NO: 29) | 47.1 |
| EAAAAARDESAYL [symbol] LQ [symbol] M [symbol] IDA [symbol] AAALSETRTIEELDTFKL [symbol] VA [symbol] FVT | |
| TVVQLAEELEHRFGRNRRGRTEIYKIVKEVDRKLLDLTDAVLAKEKKGED | |
| ILNMVAEIKALLINIYK | |
| GGGGSSGGGGSSGGGGSSGGGGSS | |
| EAAAAARDESAYL [symbol] LQ [symbol] M [symbol] IDA [symbol] AAALSETRTIEELDTFKL [symbol] VA [symbol] FVT | |
| TVVQLAEELEHRFGRNRRGRTEIYKIVKEVDRKLLDLTDAVLAKEKKGED | |
| ILNMVAEIKALLINIYK | |
| >moevan_st2 (SEQ ID NO: 30) | 8.89 |
| EAAAAARDESAYL [symbol] LQ [symbol] M [symbol] IDA [symbol] AAALSETRTIEELDTFKL [symbol] VA [symbol] FVT | |
| TVVQLAEELEHRFGRNRRGRTEIYKIVKEVDRKLLDLTDAVLAKEKKGED | |
| ILNMVAEIKALLINIYK | |
| GGGGSS | |
| EAAAAARDESAYL [symbol] LQ [symbol] M [symbol] IDA [symbol] AAALSETRTIEELDTFKL [symbol] VA [symbol] FVT | |
| TVVQLAEELEHRFGRNRRGRTEIYKIVKEVDRKLLDLTDAVLAKEKKGED | |
| ILNMVAEIKALLINIYK | |
| >sohair2_15r (SEQ ID NO: 31) | 228 |
| MTSDYIIEQIQRKQEEARL [symbol] VE [symbol] E [symbol] LEAVKEASKRGVSSDQLLNLIL [symbol] L | |
| A [symbol] IITTLIQIIEESNEAIKELIKNQ | |
| GGGGSGGGGSGGGGS | |
| DYIIEQIQRKQEEARLK [symbol] E [symbol] E [symbol] LEAVKEASKRGVSSDQLLNLIL [symbol] LA [symbol] II | |
| TTLIQIIEESNEAIKELIKNQ | |
| >bika1 (SEQ ID NO: 32) | 63 |
| SKEVLEQSLFL [symbol] LD [symbol] V [symbol] LLA [symbol] IHAIKIDRITGNMDKQKLDTAYL [symbol] VA [symbol] IE | |
| TTLYQLIEVSH | |
| >bika2 (SEQ ID NO: 33) | 98 |
| SKEVLEQSLFL [symbol] LD [symbol] V [symbol] LLA [symbol] IHAIKIDRITGNMDKQKLDTLYL [symbol] VA [symbol] IE | |
| TTLYQLIEVSH | |

TABLE 8
CoMAND ensemble structure statistics
R-factors¹

| | |
|---|---|
| Rₑₙₛ | 0.33 |
| Rₘₑₐₙ | 0.36 ± 0.11 |
| Coverage² | 89/115 |

Structure Quality

| | |
|---|---|
| Bonds (Å × 10⁻³) | 1.94 ± 0.10 |
| Angles (°) | 0.52 ± 0.02 |
| Impropers (°) | 0.83 ± 0.12 |
| Ramachandran Map (%) | 97.2/2.0/0.8 |
| Sidechain Regularity (%) | 98.1 |
| Clash Score | 0 |
| Number of Structures | 12 |
| Ordered Residues | 2-53, 60-118 |
| Backbone Heavy Atom | 1.34 ± 0.44 |
| All Heavy Atom | 1.67 ± 0.42 |

¹R-factors averaged across the sequence (±SD) are given for the final ensemble compiled by global optimization (Rmean).
²The coverage refers to the number of residue used in factorization analysis, versus the total number expected from the sequence, excluding purification tags.
³Determined by MOLPROBITY. The Ramachandran statistic lists the percentage of residues in favored/allowed/disfavored regions of the map (percentiles 98.0/99.8/>99.8). Sidechain regularity lists the percentage in allowed sidechain rotamers (percentile 98.0). The clash score lists steric overlaps > 0.4 Å per 1000 atoms.
⁴The RMSD to the average structure based on superimposition over ordered residues, as defined in the table.

TABLE 9
SPR binding parameters

| | 1:1 binding model¹ | | | |
|---|---|---|---|---|
| Analyte | kₐ (M⁻¹s⁻¹) | k_{d} (s⁻¹) | K_{d} (M) | X² (R.U.²) |
| rhG-CSF | 7.9 × 10⁵ | 2.8 × 10⁻⁴ | 3.6 × 10⁻¹⁰ | 4.3 |
| | 6.4 × 10⁵ | 2.6 × 10⁻⁴ | 4.1 × 10⁻¹⁰ | 2.8 |
| Boskar3 | 1.3 × 10⁵ | 1.5 × 10⁻³ | 1.2 × 10⁻⁸ | 2.0 |
| | 1.2 × 10⁵ | 1.9 × 10⁻³ | 1.6 × 10⁻⁸ | 1.7 |
| Boskar4 | 5.2 × 10⁵ | 4.5 × 10⁻³ | 8.5 × 10⁻⁹ | 2.1 |
| | 8.9 × 10⁵ | 1.5 × 10⁻³ | 1.7 × 10⁻⁹ | 1.6 |
| | 2^{nd} order kinetics model² | | | |
| Analyte | kₐ (M⁻¹s⁻¹) | k_{d} (s⁻¹) | K_{d} (M) | X² (R.U.²) |
| rhG-CSF | 1.1 × 10⁴ | 9.0 × 10⁻⁴ | 8.2 × 10⁻⁸ | 2.5 |
| | 1.0 × 10⁴ | 8.4 × 10⁻⁴ | 8.8 × 10⁻⁸ | 2.3 |
| Boskar3 | 7.8 × 10² | 4.7 × 10⁻³ | 6.0 × 10⁻⁶ | 1.0 |
| | 2.2 × 10³ | 5.8 × 10⁻³ | 2.6 × 10⁻⁶ | 1.1 |
| Boskar4 | 1.5 × 10³ | 9.5 × 10⁻³ | 6.4 × 10⁻⁶ | 1.8 |
| | 1.4 × 10³ | 8.0 × 10⁻³ | 5.8 × 10⁻⁶ | 1.0 |

¹Analysis was done using the Biacore ×100 evaluation software v.2.0.1.
²Analysis was done using a second-order model.

TABLE 10
SPR binding parameters

| | 1:1 binding model¹ | | | |
|---|---|---|---|---|
| Analyte | kₐ (M⁻¹s⁻¹) | k_{d} (s⁻¹) | K_{d} (M) | X² (R.U.²) |
| rhG-CSF | (3.0 ± 0.3) × | (4.9 ± 2.8) × | (1.1 ± 1.6) × | 4.9 |
| | 10⁵ | 10⁻⁴ | 10⁻⁹ | |
| Moevan | (2.9 ± 0.4) × | (5.9 ± 0.4) × | (2.1 ± 0.4) × | 0.7 |
| | 10⁵ | 10⁻³ | 10⁻⁸ | |
| Moevan_t2 | (3.1 ± 0.3) × | (3.0 ± 3.2) × | (1.1 ± 1.1) × | 0.1 |
| | 10⁵ | 10⁻⁴ | 10⁻⁹ | |
| diSohair2 | (2.1 ± 0.1) × | (9.5 ± 0.1) × | (4.5 ± 0.3) × | 1.9 |
| | 10³ | 10⁻³ | 10⁻⁶ | |

¹Analysis was done using the Biacore ×100 evaluation software v.2.0.1.

While aspects of the invention are illustrated and described in detail in the Figures and in the foregoing tables and description, such Figures, tables and description are to be considered illustrative or exemplary and not restrictive. Also reference signs in the claims should not be construed as limiting the scope.

It will also be understood that changes and modifications may be made by those of ordinary skill within the scope and spirit of the claims. In particular, the present invention covers further embodiments with any combination of features from different embodiments described above. It is also to be noted in this context that the invention covers all further features shown in the figures individually, although they may not have been described in the previous or following description. Also, single alternatives of the embodiments described in the figures and the description and single alternatives of features thereof can be disclaimed from the subject matter according to aspects of the invention.

Whenever the word “comprising” is used in the claims, it should not be construed to exclude other elements or steps. It should also be understood that the terms “essentially”, “substantially”, “about”, “approximately” and the like used in connection with an attribute or a value may define the attribute or the value in an exact manner in the context of the present disclosure. The terms “essentially”, “substantially”, “about”, “approximately” and the like could thus also be omitted when referring to the respective attribute or value. The terms “essentially”, “substantially”, “about”, “approximately” when used with a value may mean the value ±10%, preferably ±5%.

A number of documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

1. A protein comprising:
a) one or two polypeptide chains;
b) a bundle of four α-helices; and
c) two or three amino acid linkers that connect contiguous bundle-forming α-helices that are located on the same polypeptide chain, wherein each amino acid linker has a length between 2 and 15 amino acids;
wherein the protein comprises one or more G-CSF receptor (G-CSF-R) binding sites;
and wherein the protein has a melting temperature (Tₘ) of at least 74° C.

2. The protein according to claim 1, wherein each G-CSF receptor binding site individually comprises six to eight amino acid residues having a similar structure and a similar spatial orientation towards each other as the amino acid residues Lysine 16, Glutamate 19, Glutamine 20, Arginine 22, Lysine 23, Aspartate 27, Aspartate 109, and Aspartate 112 of human G-CSF.

3. The protein according to claim 1, wherein the protein
a) binds to G-CSF-R with an affinity of less than 10 μM; and/or
b) has G-CSF-like activity, in particular wherein the G-CSF-like activity comprises at least one, preferably at least two, more preferably at least three, most preferably all of the following activities:
(i) induction of granulocytic differentiation of HSPCs;
(ii) induction of the formation of myeloid colony-forming units from HSPCs;
(iii) induction of the proliferation of NFS-60 cells; and/or
(iv) activation of the downstream signaling pathways MAPK/ERK and/or JAK/STAT; and/or
c) induces the proliferation of NFS-60 cells, in particular wherein the protein induces the proliferation of NFS-60 at a half maximal effective concentration (EC50) of less than 100 μg/mL; and/or
d) induces the proliferation and/or differentiation of cells comprising one or more G-CSF receptor on the cell surface, in particular wherein the cell is a hematopoietic stem cell or a cell deriving thereof, more preferably wherein the cell is a common myeloid progenitor or a cell deriving thereof, even more preferably wherein the cell is a myeloblast or a cell deriving thereof.

4-8. (canceled)

9. The protein according to claim 1, wherein the calculated contact order number of said protein is lower than the calculated contact order number of human G-CSF (SEQ ID NO:1); and/or wherein the protein has a molecular mass between 13 and 18 kDa; and/or wherein the protein comprises no disulfide bonds; and/or wherein the protein comprises no disulfide bonds and/or wherein the protein is not glycosylated.

10-12. (canceled)

13. The protein according to claim 1, wherein the α-helices that form the bundle of four α-helices are located on a single polypeptide chain, in particular wherein the single polypeptide chain comprises a four-helix bundle arrangement, in particular wherein the four-helix bundle arrangement has an up-down-up-down topology.

14-15. (canceled)

16. The protein according to claim 13, wherein the single polypeptide chain comprises an amino acid sequence having at least 60%, 70%, 80%, 90% amino acid sequence identity with an amino acid sequence selected from the group consisting of: SEQ ID NO:5, SEQ ID NO: 4, SEQ ID NO:3, SEQ ID NO:2, SEQ ID NO:6, SEQ ID NO:14, SEQ ID NO:22 and SEQ ID NO:25; in particular wherein the single polypeptide chain comprises an amino acid sequence selected from the group consisting of: SEQ ID NO:5, SEQ ID NO: 4, SEQ ID NO:3, SEQ ID NO:2, SEQ ID NO:6, SEQ ID NO:14, SEQ ID NO:22 and SEQ ID NO:25.

17. (canceled)

18. The protein according to claim 1, wherein the α-helices that form the bundle of four α-helices are located on two separate polypeptide chains, in particular wherein each of the two polypeptide chains contributes two α-helices to the bundle of four α-helices and/or wherein each of the two polypeptide chains comprises a helical-hairpin motif; and/or wherein the two polypeptide chains form a dimer.

19-21. (canceled)

22. The protein according to claim 18, wherein both polypeptide chains comprise an amino acid sequence having at least 60%, 70%, 80%, 90% amino acid sequence identity with an amino acid sequence selected from the group consisting of: SEQ ID NO:19, SEQ ID NO:18, SEQ ID NO:32 and SEQ ID NO:33; in particular wherein both polypeptide chains comprise an amino acid sequence selected from the group consisting of: SEQ ID NO:19, SEQ ID NO:18, SEQ ID NO:32 and SEQ ID NO:33.

23. (canceled)

24. The protein according to claim 1, wherein the spatial orientation and molecular interaction features of at least two, at least three, at least four, at least five, at least six, at least seven of the amino acid residues Lysine 16, Glutamate 19, Glutamine 20, Arginine 22, Lysine 23, Aspartate 27, Asparagine 109, and Aspartate 112 of human G-CSF (SEQ ID NO:1) are preserved.

25. The protein according to claim 1, wherein the protein comprises or consists of an amino acid sequence having at least 60%, 70%, 80%, 90%, 95%, 96%, 97%, 98%, 99%, or 100% amino acid sequence identity with the amino acid sequence of SEQ ID NO:5,
wherein the protein comprises one or more G-CSF receptor (G-CSF-R) binding sites;
and wherein the protein has a melting temperature (Tₘ) of at least 75° C.

26-35. (canceled)

36. The protein according to claim 1, wherein the protein comprises or consists of an amino acid sequence having at least 60%, 70%, 80%, 90%, 95%, 96%, 97%, 98%, 99%, or 100% amino acid sequence identity with the amino acid sequence of SEQ ID NO:6,
wherein the protein comprises one or more G-CSF receptor (G-CSF-R) binding sites;
and wherein the protein has a melting temperature (Tₘ) of at least 74° C., in particular wherein the protein binds to G-CSF-R with an affinity of less than 10 μM.

37-46. (canceled)

47. The protein according to claim 1, wherein the protein comprises or consists of an amino acid sequence having at least 60%, 70%, 80%, 90%, 95%, 96%, 97%, 98%, 99%, or 100% amino acid sequence identity with the amino acid sequence of SEQ ID NO:14,
wherein the protein comprises one or more G-CSF receptor (G-CSF-R) binding sites;
and wherein the protein has a melting temperature (Tₘ) of at least 75° C., in particular wherein the protein binds to G-CSF-R with an affinity of less than 10 μM.

48-57. (canceled)

58. The protein according to claim 1, wherein the protein comprises an amino acid sequence having at least 60%, 70%, 80%, 90%, 95%, 96%, 97%, 98%, 99%, or 100% amino acid sequence identity with the amino acid sequence of SEQ ID NO:19,
wherein the protein comprises one or more G-CSF receptor (G-CSF-R) binding sites;
and wherein the protein has a melting temperature (Tₘ) of at least 75° C., in particular wherein the protein comprises two polypeptide chains, preferably wherein the two polypeptide chains of the protein comprise identical amino acid sequences, in particular wherein the protein binds to G-CSF-R with an affinity of less than 10 μM.

59-68. (canceled)

69. The protein according to claim 1, wherein the protein comprises an amino acid sequence having at least 60%, 70%, 80%, 90%, 95%, 96%, 97%, 98%, 99%, or 100% amino acid sequence identity with the amino acid sequence of SEQ ID NO:32, wherein the protein comprises one or more G-CSF receptor (G-CSF-R) binding sites;
and wherein the protein has a melting temperature (Tₘ) of at least 75° C., in particular wherein the protein comprises two polypeptide chains, preferably wherein the two polypeptide chains of the protein comprise identical amino acid sequences, in particular wherein the protein binds to G-CSF-R with an affinity of less than 10 μM.

70-79. (canceled)

80. A fusion protein comprising a first protein domain and a second protein domain, wherein the first protein domain and/or the second protein domain comprises a protein according to claim 1.

81. The fusion protein according to claim 80, wherein the first protein domain and the second protein domain are linked by a peptide linker, in particular wherein the peptide linker is a glycine-serine linker and/or wherein the linker has a length of 5 to 50 amino acid residues and/or wherein the first protein domain and the second protein domain comprise identical amino acid sequences.

82-98. (canceled)

99. A method of treating neutropenia in a subject, the method comprising administering an effective amount of the protein according to claim 1 to the subject.

100. (canceled)

101. A method of mobilizing stem cells in a subject, the method comprising administering an effective amount of the protein according to claim 1 to the subject.

102-104. (canceled)

105. A method for proliferating and/or differentiating cells in a cell culture, the method comprising the steps of:
a) providing a plurality of cells in a cell culture;
b) contacting said cells with the protein according to claim 1.
